(19) United States
(12) Patent Application Publication
(10) Pub. No.: US 2012/0165306 A1
(43) Pub. Date: Jun. 28, 2012

(54) PYRAZINYLPYRIDINES USEFUL FOR THE TREATMENT OF PROLIFERATIVE DISEASES

(76) Inventors: Paul A. Barsanti, Pleasant Hill, CA (US); Cheng Hu, Pleasant Hill, CA (US); Cheng Hu, Menlo Park, CA (US); Keith B. Pfister, San Ramon, CA (US); Martin Sendzik, San Mateo, CA (US); James Sutton, Pleasanton, CA (US)

(21) Appl. No.: 13/393,457

(22) PCT Filed: Sep. 2, 2010
(86) PCT No.: PCT/EP2010/062881

§ 371 Date: Feb. 29, 2012

Related U.S. Application Data
(60) Provisional application No. 61/275,938, filed on Sep. 4, 2009.
(60) Provisional application No. 61/284,962, filed on Dec. 28, 2009.

(51) Int. Cl. A61K 31/497; C07D 405/14; C07D 413/14; C07D 409/14; A61P 31/18; A61K 31/553; A61K 31/55; A61P 35/00; A61P 29/00; A61P 9/00; C07D 401/14; A61K 31/5377

(52) U.S. Cl. 514/210.18; 544/405; 544/120; 544/230; 540/544; 540/598; 514/255.05; 514/235.8; 514/211.15; 514/217.05

(57) ABSTRACT

The present invention provides a compound of Formula (I):

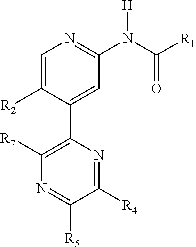

and pharmaceutically acceptable salts thereof. Also provided is a method of using a compound of Formula I for treating a disease or condition mediated by a CDK inhibitor.

### CROSS-REFERENCE TO RELATED APPLICATIONS

This application claims the benefit of priority under 35 U.S.C. §119(e) to U.S. provisional application Ser. No. 61/275,938 filed on Sep. 4, 2009, and U.S. provisional application Ser. No. 61/284,962 filed on Dec. 28, 2009, which are incorporated herein by reference in their entirety.

### FIELD OF THE INVENTION

The invention provides a novel class of compounds, pharmaceutical compositions comprising such compounds and methods of using such compounds to treat or prevent diseases or disorders associated with aberrant cellular signaling pathways that can be modulated by inhibition of kinases, particularly diseases or disorders that involve aberrant cellular signaling pathways that can be modulated by inhibition of CDK9.

### BACKGROUND

Protein kinases constitute a large family of structurally related enzymes that are responsible for the control of a variety of signal transduction processes within the cell. (Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

Many diseases are associated with abnormal cellular responses triggered by the protein kinase-mediated events described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, viral diseases, and hormone-related diseases. Accordingly, there has been a substantial effort in medicinal chemistry to find protein kinase inhibitors that are effective as therapeutic agents.

The cyclin-dependent kinase (CDK) complexes are a class of kinases that are targets of interest. These complexes comprise at least a catalytic (the CDK itself) and a regulatory (cyclin) subunit. Some of the more important complexes for cell cycle regulation include cyclin A (CDK1—also known as cdc2, and CDK2), cyclin B1-B3 (CDK1) and cyclin D1-D3 (CDK2, CDK4, CDK5, CDK6), cyclin E (CDK2). Each of these complexes is involved in a particular phase of the cell cycle. Additionally, CDKs 7, 8, and 9 are implicated in the regulation of transcription.

The CDKs seem to participate in cell cycle progression and cellular transcription, and loss of growth control is linked to abnormal cell proliferation in disease (see e.g., Malumbres and Barbacid, Nat. Rev. Cancer 2001, 1:222). Increased activity or temporally abnormal activation of cyclin-dependent kinases has been shown to result in the development of human tumors (Sherr C. J., Science 1996, 274: 1672-1677). Indeed, human tumor development is commonly associated with alterations in either the CDK proteins themselves or their regulators (Cordon-Cardo C., Am. J. Pat 1/701. 1995; 147: 545-560; Karp J. E. and Broder S., Nat. Med. 1995; 1: 309-320; Hall M. et al., Adv. Cancer Res. 1996; 68: 67-108).

CDKs 7 and 9 seem to play key roles in transcription initiation and elongation, respectively (see, e.g., Peterlin and Price. Cell 23: 297-305, 2006, Shapiro. J. Clin. Oncol. 24: 1770-83, 2006;). Inhibition of CDK9 has been linked to direct induction of apoptosis in tumor cells of hematopoetic lineages through down-regulation of transcription of antiapoptotic proteins such as Mc11 (Chao, S.-H. et al. J. Biol. Chem. 2000; 275:28345-28348; Chao, S.-H. et al. J. Biol. Chem. 2001; 276:31793-31799; Lam et. al. Genome Biology 2: 0041.1-11, 2001; Chen et al. Blood 2005; 106:2513; MacCallum et al. Cancer Res. 2005; 65:5399; and Alvi et al. Blood 2005; 105:4484). In solid tumor cells, transcriptional inhibition by downregulation of CDK9 activity synergizes with inhibition of cell cycle CDKs, for example CDK1 and 2, to induce apoptosis (Cai, D.-P., Cancer Res 2006, 66:9270. Inhibition of transcription through CDK9 or CDK7 may have selective non-proliferative effect on the tumor cell types that are dependent on the transcription of mRNAs with short half lives, for example Cyclin D1 in Mantle Cell Lymphoma. Some transcription factors such as Myc and NF-kB selectively recruit CDK9 to their promoters, and tumors dependent on activation of these signaling pathways may be sensitive to CDK9 inhibition.

Small molecule CDK inhibitors may also be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein. Moreover, the purine CDK2 inhibitor CVT-313 (Ki=95 nM) resulted in greater than 80% inhibition of neointima formation in rats.

CDKs are important in neutrophil-mediated inflammation and CDK inhibitors promote the resolution of inflammation in animal models. (Rossi, A. G. et al, Nature Med. 2006, 12:1056). Thus CDK inhibitors, including CDK9 inhibitors, may act as anti-inflammatory agents.

Certain CDK inhibitors are useful as chemoprotective agents through their ability to inhibit cell cycle progression of normal untransformed cells (Chen, et al. J. Natl. Cancer Institute, 2000; 92: 1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

Accordingly, there is a great need to develop inhibitors of protein kinases, such as CDK1, CDK2, CDK3, CDK4, CDK5, CDK6, CDK7, CDK8 and CDK9, as well as combinations thereof.

### SUMMARY

The present invention provides a compound of Formula I

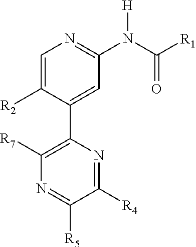

or a pharmaceutically acceptable salt thereof, wherein:

R₁ is selected from —(CH₂)₀₋₂-heteroaryl, —(CH₂)₀₋₂-aryl, C₁₋₈ alkyl, C₃₋₈ branched alkyl, C₃₋₈ cycloalkyl, and a 4 to 8 membered heterocycloalkyl group, wherein said groups are each independently optionally substituted;

R₂ is selected from hydrogen, C₁₋₄ alkoxy, C₁₋₄ haloalkyl, C₁₋₄-alkyl, and halogen;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, and C₃₋₈ branched haloalkylene;

R₈ is selected from hydrogen, C₁₋₄ alkyl, C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted; and

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl.

A preferred embodiment provides a compound of Formula I, wherein, R₁ is selected from —(CH₂)₀₋₂-heteroaryl, and —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is O, SO₂, or NR₈;

L is selected from C₀₋₃-alkylene, —CHD-, —CD-, C₃₋₆ cycloalkyl, C₃₋₆ cyclo haloalkyl, C₄₋₇-heterocycloalkyl, and C₃₋₈ branched alkylene;

R₈ is selected from hydrogen, C₁₋₄ alkyl, C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂⁻⁴ to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; alternatively, R¹⁵ and R¹⁶ along with the nitrogen atom to which they are attached can be taken together to form an optionally substituted four to six membered heteroaromatic, or non-aromatic heterocyclic ring.

A further preferred embodiment provides a compound of Formula I, wherein, R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆, heterocycloalkyl-R¹⁴, —SO₂—NR¹⁵R¹⁶ and —SO₂—C₃₋₅ cycloalkyl;

R₂ is selected from hydrogen, and halogen;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, and CF₃;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;

R¹⁴ is selected from phenyl, halogen, hydroxyl, C₁₋₂-alkyl, CF₃, and hydrogen; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

Another preferred embodiment provides a compound of Formula I, wherein, R₁ is selected from C₁₋₈ alkyl, C₃₋₈ cycloalkyl, C₃₋₈ branched alkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, and C₃₋₈ branched haloalkylene;

R₈ is selected from hydrogen, C₁₋₄ alkyl, C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; alternatively, R¹⁵ and R¹⁶ along with the nitrogen atom to which they are attached can be taken together to form an optionally substituted four to six membered heteroaromatic, or non-aromatic heterocyclic ring.

A further preferred embodiment of this aspect of the invention provides a compound of Formula I, wherein, R₁ is selected from C₁₋₈ alkyl, C₃₋₈ branched alkyl, C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is selected from hydrogen, and halogen;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, methyl, and CF₃;

R₇ is selected from hydrogen, F, Cl, and methyl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and

R¹⁴ is selected from phenyl, halogen, hydroxy, C₁₋₂-alkyl, and hydrogen.

Yet another preferred embodiment of this aspect of the invention provides a compound of Formula I, wherein, R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, and —O—CH—(CH₃)₂;

R₂ is selected from Cl, and F;

R₄ is A₆-L-R₉;

R₅ is selected from hydrogen, Cl, and methyl;

R₇ is selected from hydrogen, Cl, and methyl;

A₆ is NR₈;

L is selected from C₀₋₃-alkylene, —CD₂-, and C₃₋₈ branched alkylene;

R₈ is selected from hydrogen, and methyl; and

R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

Provided in a particularly preferred embodiment is a compound of Formula I, wherein, R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, and —O—CH—(CH₃)₂;

R₂ is Cl;

R₄ is A₆-L-R₉;

R₅ is selected from hydrogen, and methyl;

R₇ is selected from hydrogen, and methyl;

A₆ is NR₈;

L is selected from —CH₂—, and —CD₂-;

R₈ is selected from hydrogen, and methyl; and

R₉ is selected from pyridyl, benzyl, tetrahydro-pyran, dioxane, and tetrahydrofuran, wherein said groups are optionally substituted with one to three substituents each independently selected from F, OH, methyl, ethyl, methoxy, and CN.

Preferred Formula I compounds of the present invention are selected from, (S)-1-Methanesulfonyl-piperidine-3-carboxylic acid (5-chloro-4-{6-[(tetrahydro-pyran-4-ylmethyl)-amino]-pyrazin-2-yl}-pyridin-2-yl)-amide; (S)-1-Ethanesulfonyl-piperidine-3-carboxylic acid (5-chloro-4-{6-[(tetrahydro-pyran-4-ylmethyl)-amino]-pyrazin-2-yl}-pyridin-2-yl)-amide; (S)-1-(2-Methoxy-acetyl)-piperidine-3-carboxylic acid (5-chloro-4-{6-[(tetrahydro-pyran-4-ylmethyl)-amino]-pyrazin-2-yl}-pyridin-2-yl)-amide; (S)-1-Acetyl-piperidine-3-carboxylic acid (5-chloro-4-{6-[(tetrahydro-pyran-4-ylmethyl)-amino]-pyrazin-2-yl}-pyridin-2-yl)-amide; (1S,3R)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide; (1R,3S)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide; (1R,3R)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide; [(1R,3S)-3-(5-Chloro-4-{6-[(tetrahydro-pyran-4-ylmethyl)-amino]-pyrazin-2-yl}-pyridin-2-ylcarbamoyl)-cyclopentyl]-carbamic acid methyl ester; [(1S,3R)-3-(5-Chloro-4-{6-[(tetrahydro-pyran-4-ylmethyl)-amino]-pyrazin-2-yl}-pyridin-2-ylcarbamoyl)-cyclopentyl]-carbamic acid methyl ester; (S)-1-(Propane-2-sulfonyl)-piperidine-3-carboxylic acid (5-chloro-4-{6-[(tetrahydro-pyran-4-ylmethyl)-amino]-pyrazin-2-yl}-pyridin-2-yl)-amide; 1-Methyl-5-oxo-pyrrolidine-3-carboxylic acid (5-chloro-4-{6-[(tetrahydro-pyran-4-ylmethyl)-amino]-pyrazin-2-yl}-pyridin-2-yl)-amide; (R)-Piperidine-3-carboxylic acid (5-chloro-4-{6-[(tetrahydro-pyran-4-ylmethyl)-amino]-pyrazin-2-yl}-pyridin-2-yl)-amide; N-(5-Chloro-4-{6-[(tetrahydro-pyran-4-ylmethyl)-amino]-pyrazin-2-yl}-pyridin-2-yl)-isobutyramide; (R)-Piperidine-3-carboxylic acid {5-chloro-4-[6-(2-fluoro-benzylamino)-pyrazin-2-yl]-pyridin-2-yl}-amide; Tetrahydro-pyran-4-carboxylic acid (5-chloro-4-{6-[(tetrahydro-pyran-4-ylmethyl)-amino]-pyrazin-2-yl}-pyridin-2-yl)-amide; (R)-Piperidine-3-carboxylic acid (5-chloro-4-{6-[(5-fluoro-pyridin-3-ylmethyl)-amino]-pyrazin-2-yl}-pyridin-2-yl)-amide; (R)-Piperidine-3-carboxylic acid {5-chloro-4-[6-(4-fluoro-benzylamino)-pyrazin-2-yl]-pyridin-2-yl}-amide; Morpholine-2-carboxylic acid (5-chloro-4-{6-[(tetrahydro-pyran-4-ylmethyl)-amino]-pyrazin-2-yl}-pyridin-2-yl)-amide; (R)-Piperidine-3-carboxylic acid (5-chloro-4-{3-methyl-6-[(tetrahydro-pyran-4-ylmethyl)-amino]-pyrazin-2-yl}-pyridin-2-yl)-amide; (R)-Piperidine-3-carboxylic acid (5-chloro-4-{6-[(tetrahydro-pyran-3-ylmethyl)-amino]-pyrazin-2-yl}-pyridin-2-yl)-amide; (R)-Piperidine-3-carboxylic acid {5-chloro-4-[6-((R)-1-cyclohexyl-ethylamino)-pyrazin-2-yl]-pyridin-2-yl}-amide; (R)-Pyrrolidine-3-carboxylic acid (5-chloro-4-{6-[(tetrahydro-pyran-4-ylmethyl)-amino]-pyrazin-2-yl}-pyridin-2-yl)-amide; (1R,3S)-3-Amino-cyclopentanecarboxylic acid (5-chloro-4-{6-[(tetrahydro-pyran-4-ylmethyl)-amino]-pyrazin-2-yl}-pyridin-2-yl)-amide; (1R,3R)-3-Amino-cyclopentanecarboxylic acid (5-chloro-4-{6-[(tetrahydro-pyran-4-ylmethyl)-amino]-pyrazin-2-yl}-pyridin-2-yl)-amide; and (3R,4S)-4-Fluoro-pyrrolidine-3-carboxylic acid (5-chloro-4-{6-[(tetrahydro-pyran-4-ylmethyl)-amino]-pyrazin-2-yl}-pyridin-2-yl)-amide.

The present invention in one of its embodiments provides a compound of Formula I

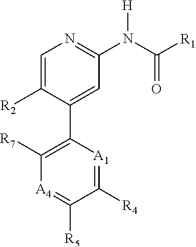

or a pharmaceutically acceptable salt thereof, wherein, R₁ is C₃₋₈ cycloalkyl, (CH₂)₁₋₂ heteroaryl, or a 4 to 8 membered heterocycloalkyl group, wherein said cycloalkyl, heteroaryl, and heterocycloalkyl groups are each independently optionally substituted with one to three substituents selected from the group consisting of —NH—C(O)—CH₂—O—C₁₋₄ alkyl, —NHC(O)—C₁₋₄ alkyl, —C(O)—O—C₁₋₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, and —NH—SO₂—C₁₋₄ alkyl; R₂ is C₁₋₄ alkoxy, or halogen; A₁ is N or CR₃; A₄ is N and CR₆, with the proviso that at least one of A₁ and A₄ is a N; R₃ is halogen, C₁₋₄ alkoxy, or hydrogen; R₄ is hydrogen, halogen, or A₆-L-R₉; R₅ is hydrogen, C₁₋₄ alkyl, or halogen; R₆ is hydrogen, or halogen; R₇ is hydrogen, C₁₋₄ alkyl, or halogen; A₆ is NR_{S}; L is C₁₋₃-alkylene or C₃₋₈ branched alkylene; R₈ is hydrogen, or C₁₋₄ alkyl; and R₉ is hydrogen, 4 to 8 member heterocycloalkyl, heteroaryl, or aryl, wherein the heterocycloaklyl, heteroaryl, and aryl groups are optionally substituted with one to three substituents each independently selected from halogen, C₁₋₄ alkyl, or C₁₋₄ haloalkyl.

A preferred embodiment provides a compound of Formula I, or a pharmaceutically acceptable salt thereof, wherein, R₁ is C₃₋₈ cycloalkyl, —(CH₂)₁₋₂ heteroaryl, or a 4 to 8 membered heterocycloalkyl group, wherein said cycloalkyl, heteroaryl, and heterocycloalkyl groups are optionally substituted with one to three substituents each independently selected from —NH—C(O)—CH₂—O—C₁₋₄ alkyl, —NHC(O)—C₁₋₄ alkyl, —C(O)—O—C₁₋₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, or —NH—SO₂—C₁₋₄ alkyl; R₂ is C₁₋₄ alkoxy, or halogen; A₁ is N; A₄ is CR₆; R₄ is hydrogen, halogen, or A₆-L-R₉; R₅ is hydrogen, C₁₋₄ alkyl, or halogen; R₆ is hydrogen, or halogen; R₇ is hydrogen, C₁₋₄ alkyl, or halogen; A₆ is NR₈; L is C₁₋₃-alkylene or C₃₋₈ branched alkylene; R₈ is hydrogen, or C₁₋₄ alkyl; and R₉ is hydrogen, 4 to 8 member heterocycloalkyl, heteroaryl, or aryl, wherein the heterocycloaklyl, heteroaryl, and aryl groups are optionally substituted with one to three substituents each independently selected from halogen, C₁₋₄ alkyl, or C₁₋₄ haloalkyl.

A further preferred embodiment provides a compound of Formula I, wherein, R₁ is cyclohexyl or piperidinyl wherein said cyclohexyl and said piperidinyl are each optionally substituted with one to two substituents each independently selected from a group consisting of —NHC(O)—C₁₋₄ alkyl, —C(O)—O—C₁₄alkyl, —C(O)—CH₂—O—C₁₋₄ alkyl, —C₁₋₄ alkyl, —(CH₂)₁₋₃—O—C₁₋₂ alkyl, —SO₂—C₁₋₄ alkyl, —NH—C(O)—C₁₋₄ alkyl, and —NH—SO₂—C₁₋₄ alkyl; R₂ is halogen; R₄ is hydrogen, or A₆-L-R₉; R₅ is methyl, hydrogen, or halogen; R₆ is —OCH₃, hydrogen, or halogen; R₇ is hydrogen, or halogen; A₆ is NR₈; L is —CH₂—, or C₃₋₆ branched alkylene; R₈ is methyl or hydrogen; and R₉ is tetrahydropyran, or phenyl, wherein said tetrahydropyran and phenyl groups are optionally substituted with one to two substituents each independently selected from halogen, or C₁₋₂-alkyl.

Provided in another preferred embodiment is a compound of Formula I, or a pharmaceutically acceptable salt thereof, wherein, R₁ represents C₃₋₈ cycloalkyl, —(CH₂)₁₋₂ heteroaryl, or a 4 to 8 membered heterocycloalkyl group, wherein said cycloalkyl, heteroaryl, and heterocycloalkyl groups are optionally substituted with one to three substituents each independently selected from —NH—C(O)—CH₂—O—C₁₋₄ alkyl, —NHC(O)—C₁₋₄ alkyl, —C(O)—O—C₁₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, and —NH—SO₂—C₁₋₄ alkyl; R₂ is C₁₋₄ alkoxy, or halogen; A₁ is CR₃; A₄ is N; R₃ is halogen, C₁₋₄ alkoxy, or hydrogen; R₄ is hydrogen, halogen, or A₆-L-R₉; R₅ is hydrogen, C₁₋₄ alkyl, or halogen; R₇ is hydrogen, C₁₋₄ alkyl, or halogen; A₆ is NR₈; L is C₁₋₃-alkylene or C₃₋₈ branched alkylene; R₈ is hydrogen, or C₁₋₄ alkyl; R₉ is hydrogen, 4 to 8 member heterocycloalkyl, heteroaryl, or aryl, wherein the heterocycloaklyl, heteroaryl, and aryl groups are optionally substituted with one to three substituents each independently selected from halogen, C₁₋₄ alkyl, or C₁₋₄ haloalkyl.

Another preferred embodiment provides compounds of Formula I, wherein, R₁ is cyclohexyl or piperidinyl wherein said cyclohexyl and said piperidinyl are each optionally substituted with one to two substituents selected from a group consisting of —NHC(O)—C₁₋₄ alkyl, —C(O)—O—C₁₄alkyl, —C(O)—CH₂—O—C₁₋₄ alkyl, —C₁₋₄ alkyl, —(CH₂)₁₋₃—O—C₁₋₂ alkyl, —SO₂—C₁₋₄ alkyl, —NH—C(O)—C₁₋₄ alkyl, and —NH—SO₂—C₁₋₄ alkyl; R₂ is halogen; R₄ is hydrogen or A₆-L-R₉; R₅ is methyl, hydrogen, or halogen; R₆ is hydrogen or halogen; R₇ is hydrogen or halogen; A₆ is NR₈; L is —CH₂— or C₃₋₆ branched alkylene; R₈ is methyl or hydrogen; and R₉ is tetrahydropyran, or phenyl, wherein said tetrahydropyran and phenyl groups are optionally substituted with one to two substituents each independently halogen, or C₁₋₂-alkyl.

Another embodiment provides a method of treating a disease or condition mediated by CDK9 by using a compound of Formula I or a pharmaceutically acceptable salt thereof. Also provided in another embodiment is the manufacture of a medicament for the treatment of a disease or condition mediated by CDK9, said medicament comprising a compound of Formula I or a pharmaceutically acceptable salt thereof.

Another aspect of the present invention provides a method of treating a disease or condition mediated by CDK9 using compound of Formula I or pharmaceutically acceptable salt thereof. A preferred method comprises using a therapeutically effective amount of a compound of Formula I.

The present invention also provides a pharmaceutical composition comprising a compound of Formula I, or a pharmaceutically acceptable salt thereof, and a pharmaceutically acceptable carrier, diluent or excipient. Also provided in another embodiment is the use of a compound of Formula I, or a pharmaceutically acceptable salt thereof in the manufacture of a medicament for the treatment of a disease or condition mediated by CDK9.

In another aspect, the present invention provides a method of regulating, modulating, or inhibiting protein kinase activity which comprises contacting a protein kinase with a compound of the invention. Suitable protein kinases include CDK1, CDK2, CDK3, CDK4, CDK5, CDK6, CDK7, CDK8 and CDK9, or any combination thereof. Preferably, the protine kinase is selected from the group consisting of CDK1, CDK2 and CDK9, or any combination thereof. In still another embodiment, the protein kinase is in a cell culture. In yet another embodiment, the protein kinase is in a mammal.

In another aspect, the invention provides a method of treating a protein kinase-associated disorder comprising administering to a subject in need thereof a pharmaceutically acceptable amount of a compound of the invention. Suitable protein kinases include CDK1, CDK2, CDK3, CDK4, CDK5, CDK6, CDK7, CDK8 and CDK9 or combinations thereof (preferably, the protein kinase is selected from the group consisting of CDK1, CDK2 and CDK9, more preferably, the protein kinase is CDK9.) Suitable CDK combinations include CDK4 and CDK9; CDK1, CDK2 and CDK9; CDK9 and CDK7; CDK9 and CDK1; CDK9 and CDK2; CDK4, CDK6 and CDK9; CDK1, CDK2, CDK3, CDK4, CDK6 and CDK9.

In yet another aspect, the invention provides a method of treating cancer comprising administering to a subject in need thereof a pharmaceutically acceptable amount of a compound of the invention. Suitable cancers for treatment includebladder, head and neck, breast, stomach, ovary, colon, lung, brain, larynx, lymphatic system, hematopoetic system, genitourinary tract, gastrointestinal, ovarian, prostate, gastric, bone, small-cell lung, glioma, colorectal and pancreatic cancer.

### DEFINITIONS

As used herein, the term “protein kinase-associated disorder” includes disorders and states (e.g., a disease state) that are associated with the activity of a protein kinase, e.g., the CDKs, e.g., CDK1, CDK2 and/or CDK9. Non-limiting examples of protein kinase-associated disorders include abnormal cell proliferation (including protein kinase-associated cancers), viral infections, fungal infections, autoimmune diseases and neurodegenerative disorders.

The term “treat,” “treated,” “treating” or “treatment” includes the diminishment or alleviation of at least one symptom associated or caused by the state, disorder or disease being treated. In certain embodiments, the treatment comprises the induction of a protein kinase-associated disorder, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

The term “use” includes one or more of the following embodiments of the invention, respectively: the use in the treatment of protein kinase-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from cancer, inflammation, cardiac hypertrophy, and HIV infection, as well as those diseases that depend on the activity of protein kinases. The term “use” further includes embodiments of compositions herein which bind to a protein kinase sufficiently to serve as tracers or labels, so that when coupled to a fluor or tag, or made radioactive, can be used as a research reagent or as a diagnostic or an imaging agent.

The term “alkyl,” by itself or as part of another substituent, means, unless otherwise stated, a fully saturated straight-chain (linear; unbranched) or branched chain, having the number of carbon atoms specified, if designated (i.e. C₁-C₁₀ means one to ten carbons). Illustrative “alkyl” group examples are methyl, ethyl, n-propyl, isopropyl, n-butyl, t-butyl, isobutyl, sec-butyl, n-pentyl, n-hexyl, n-heptyl, n-octyl, and the like. If no size is designated, the alkyl groups mentioned herein contain 1-10 carbon atoms, typically 1-8 carbon atoms, and preferably 1-6 or 1-4 carbon atoms.

The terms “alkoxy,” refers to —O-alkyl, wherein the term alkyl is as defined above.

The term “cycloalkyl” by itself or in combination with other terms, represents, unless otherwise stated, cyclic versions of alkyl. Additionally, cycloalkyl may contain fused rings, but excludes fused aryl and heteroaryl groups. Cycloalkyl groups, unless indicated otherwise, are unsubstituted. Illustrative examples of cycloalkyl are cyclopropyl, cyclobutyl, cyclopentyl, cyclohexyl, cycloheptyl, norbornyl, and the like. If no ring size is specified, the cycloalkyl groups described herein generally contain 3-10 ring members, preferably 3-6 ring members.

The term “heterocyclic” or “heterocycloaklyl” or “heterocyclyl,” by itself or in combination with other terms, represents a cycloalkyl containing at least one annular carbon atom and at least one annular heteroatom selected from the group consisting of O, N, P, Si and S, preferably from N, O and S, wherein the ring is not aromatic but can contain unsaturations. The nitrogen and sulfur atoms in a heterocyclic group may optionally be oxidized and the nitrogen heteroatom may optionally be quaternized. The heterocyclic groups discussed herein, if not otherwise specified, contain 3-10 ring members, and at least one ring member is a heteroatom selected from N, O, P, Si, and S. Preferably, not more than three of these heteroatoms are included in a heterocyclic group, and generally not more than two of these heteroatoms are present in a single ring of the heterocyclic group. The heterocyclic group can be fused to an additional carboclic or heterocyclic ring. A heterocyclic group can be attached to the remainder of the molecule at an annular carbon or annular heteroatom. Additionally, heterocyclic may contain fused rings, but excludes fused systems containing a heteroaryl group as part of the fused ring system. Illustrative examples of heterocyclic groups include, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, piperidin-2-one, azepane, tetrahydro-2H-pyranyl, pyrrolidinyl, methylpyrrolidinone, alkylpiperidinyl, haloalkylperidinyl, 1-(alkylpiperidin-1-yl)ethanone, and the like.

The term “aryl”, unless otherwise stated, represents an aromatic hydrocarbon group which can be a single ring or multiple rings (e.g., from 1 to 3 rings) which are fused together. Aryl includes fused rings, wherein one or more of the fused rings is fully saturated (e.g., cycloalkyl) or partially unsaturated (e.g., cyclohexenyl), but not a heterocyclic or heteroaromatic ring. Illustrative examples of aryl groups include, but are not limited to, phenyl, 1-naphthyl, 2-naphthyl, and tetrahydronaphthyl.

The term “heteroaryl”, as used herein, refers to groups comprising a single ring, or a fused ring, where at least one of the rings is an aromatic ring that contain from one to four heteroatoms selected from N, O, and S as ring members (i.e., it contains at least one heteroaromatic ring), wherein the nitrogen and sulfur atoms can be oxidized, and the nitrogen atom(s) can be quaternized. A heteroaryl group can be attached to the remainder of the molecule through an annular carbon or annular heteroatom, and it can be attached through any ring of the heteroaryl moiety, if that moiety is a bicyclic, tricyclic, or a fused ring system. A heteroaryl group may contain fused rings, wherein one of the fused rings is aromatic or heteroaromatic, and the other fused ring(s) are partially unsaturated (e.g., cyclohexenyl, 2,3-dihydrofuran, tetrahydropyrazine, and 3,4-dihydro-2H-pyran), or completely saturated (e.g., cyclohexyl, cyclopentyl, tetrahydrofuran, morpholine, and piperazine). The term heteroaryl is also intended to include fused rings systems that include a combination of aromatic and heteroaromatic rings systems (e.g., indoles, quinoline, quinazolines, and benzimidazoles). Illustrative examples of heteroaryl groups are 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

The terms “halo” or “halogen,” represents a fluorine, chlorine, bromine, or iodine atom. The term “haloalkyl,” represents an alkyl group as defined above, wherein one or more hydrogen atoms of the alkyl group are replaced by a halogen atom which may be the same or different. The term haloalkyl thus includes mono-haloalkyl, di-haloalkyl, tri-haloalkyl, tetra-haloalkyl, and the like as well as per-haloalkyl. The prefix “perhalo” refers to the respective group wherein all available valences are replaced by halo groups. For example “perhaloalkyl” includes —CCl₃, —CF₃, —CCl₂CF₃, and the like. The terms “perfluoroalkyl” and “perchloroalkyl” are a subset of perhaloalkyl wherein all available valences are replaced by fluoro and chloro groups, respectively. Illustrative examples of perfluoroalkyl include —CF₃ and —CF₂CF₃, and of perchloroalkyl include —CCl₃ and —CCl₂CCl₃.

“Optionally substituted” as used herein indicates that the particular group or groups being described may have no non-hydrogen substituents (i.e., it can be unsubstituted), or the group or groups may have one or more non-hydrogen substituents. If not otherwise specified, the total number of such substituents that may be present is equal to the number of H atoms present on the unsubstituted form of the group being described. Typically, an optionally substituted group will contain up to four (1-4) substituents. Where an optional substituent is attached via a double bond, such as a carbonyl oxygen (═O), the group takes up two available valences on the group being substituted, so the total number of substituents that may be included is reduced according to the number of available valences. Suitable optional substituent groups include halo, C₁₋₄alkyl, —NH—C(O)—CH₂—O—C₁₋₄ alkyl, —NHC(O)—C₁₋₄ alkyl, —C(O)—O—C₁₋₄alkyl, —O—C₁₋₄alkyl, —O—C₁₋₄haloalkyl, —C₁₋₄alkylene-O—C₁₋₄haloalkyl, —C₁₋₄alkylene-O—C₁₋₄alkyl, —NH—C₁₋₄alkyl, —C(O)—CH₂—O—C₁₋₄ alkyl, —C(O)—O—C₃₋₆ branched alkyl, —C₁₋₄ haloalkyl, —(CH₂)₁₋₃—O—C₁₋₂ alkyl, —C₁₋₄-cycloalkyl, —C₁₋₄alkylene-O—C₁₋₄alkyl, —NH₂, —SO₂—C₁₋₄alkyl, —NH—C(O)—C₁₋₄ alkyl, and —NH—SO₂—C₁₋₄ alkyl, hydroxyl, nitro, cyano, oxo, —C(O)—C₁₋₄alkyl, —C(O)— and the like.

“Unless specified otherwise, the term “compounds of the present invention” refer to compounds of Formula I, prodrugs thereof, pharmaceutically acceptable salts of the compounds, and/or prodrugs, and hydrates or solvates of the compounds, salts, and/or prodrugs, as well as, all stereoisomers (including diastereoisomers and enantiomers), tautomers, and isotopically labeled compounds (including deuterium substitutions), as well as inherently formed moieties (e.g., polymorphs, solvates and/or hydrates).

As used herein, the term “pharmaceutically acceptable salts” refers to salts that retain the biological effectiveness and properties of the compounds of this invention and, which typically are not biologically or otherwise undesirable.

The term “a therapeutically effective amount” of a compound of the present invention refers to an amount of the compound of the present invention that when administered to a subject, is effective to (1) at least partially alleviating, inhibiting, preventing and/or ameliorating a condition, or a disorder or a disease (i) mediated by one or more CDK enzymes, or (ii) associated with one or more CDK enzyme activities, or (iii) characterized by activity of proteins regulated (directly or indirectly) by one or more CDK enzymes (e.g. RNA polymerase II); or (2) reducing or inhibiting the expression of proteins whose expression is dependent (directly or indirectly) on one or more CDK enzymes (e.g. Mc1-1, Cyclin D, Myc etc.). When used in conjunction with a cell, the term “a therapeutically effective amount” refers to the amount of the compound of the present invention that, when administered to a cell, or a tissue, or a non-cellular biological material, or a medium, is effective to at least partially reducing or inhibiting the activity of proteins regulated by one or more CDK enzymes; or at least partially reducing or inhibiting the expression of proteins whose expression is dependent (directly or indirectly) on one or more CDK enzymes.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

Unless defined otherwise or clearly indicated by context, all technical and scientific terms used herein have the same meaning as commonly understood by one of ordinary skill in the art to which this invention belongs.

### DETAILED DESCRIPTION

The compounds disclosed herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine experimentation.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

The various starting materials, intermediates, and compounds of the embodiments may be isolated and purified, where appropriate, using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention (e.g., formulas I or II), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

Compounds of the embodiments may generally be prepared using a number of methods familiar to one skilled in the art.

The compounds of the presention invention can be isolated and used per se or as their pharmaceutical acceptable salt. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns Ito XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods.

Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The compounds of the present invention also include isotopically labeled forms of the compounds which may be synthesized using the processes described herein or modifications thereof known by those of skill in the art. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵ N, ¹⁸F, ³¹P, ³²P, ³⁵S, ²⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Compounds of the present invention include isomers including all stereoisomers of the compounds referred to in the formulas herein, including enantiomers, diastereomers, as well as all conformers, rotamers, and tautomers, unless otherwise indicated. The invention includes all enantiomers of any chiral compound disclosed, in either substantially pure levorotatory or dextrorotatory form, or in a racemic mixture, or in any ratio of enantiomers.

Furthermore, the compounds disclosed herein may contain one or more chiral centers. Accordingly, if desired, such compounds can be prepared or isolated as pure stereoisomers, i.e., as individual enantiomers or diastereomers, or as stereoisomer enriched mixtures. All such stereoisomers (and enriched mixtures) are included within the scope of the embodiments, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless stereochemistry is explicitly indicated in a chemical structure or chemical name, the chemical structure or chemical name is intended to embrace all possible stereoisomers, conformers, rotamers, and tautomers of the compound depicted. For example, a compound containing a chiral carbon atom is intended to embrace both the (R) enantiomer and the (S) enantiomer, as well as mixtures of enantiomers, including racemic mixtures; and a compound containing two chiral carbons is intended to embrace all enantiomers and diastereomers (including (R,R), (S,S), (R,S), and (R,S) isomers).

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. As defined herein, solvates and hydrates of the compounds of the present invention are considered compositions, wherein the composition comprises a compound of the present invention and a solvent (including water).

The compounds of the present invention may exist in either amorphous or polymorphic form; therefore, all physical forms are considered to be within the scope of the present invention.

Compounds of the invention, i.e. compounds of the present invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

In certain uses of the compounds of the present invention, it may be advantageous to use a pro-drug of the compound. In general, pro-drugs convert in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Typically, the compounds of the present invention are administered as a pharmaceutical composition. A typical pharmaceutical composition comprises a compound of the present invention and a pharmaceutically acceptable carrier, diluent or excipient. As used herein, the term “pharmaceutically acceptable carriers, diluents or excipients” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical composition can be formulated for particular routes of administration such as oral administration, and parenteral administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.
   Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

The invention further provides pharmaceutical compositions and dosage forms that may comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of Formula I in free form or in pharmaceutically acceptable salt form, exhibit valuable pharmacological properties, e.g. CDK inhibiting properties, e.g. as indicated in in vitro and in vivo tests as provided below and are therefore indicated for therapy.

When used with respect to methods of treatment/prevention and the use of the compounds and formulations thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.). Typically, when a step of administering a compound of the invention is disclosed herein, the invention further contemplates a step of identifying an individual or subject in need of the particular treatment to be administered or having the particular condition to be treated.

### EXAMPLES

Referring to the examples that follow, compounds of the embodiments were synthesized using the methods described herein, or other methods known to one skilled in the art. The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18 5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on LCMS instruments: Waters System (Acuity HPLC and a Micromass ZQ mass spectrometer; Column: Acuity HSS C18 1.8-micron, 2.1×50 mm; gradient: 5-95% acetonitrile in water with 0.05% TFA over a 1.8 min period; flow rate 1.2 mL/min; molecular weight range 200-1500; cone Voltage 20 V; column temperature 50° C.). All masses were reported as those of the protonated parent ions.

### Specific Optical Rotation

The specific optical rotation was measured on an Autopol IV Automatic Polarimeter (Rudolph Research Analytical) with a 100-mm path-length cylindrical glass cell at 20° C. temperature. The wavelength of the light used was 589 nanometer (the sodium D line). Optical rotation of the same cell filled with solvent was subtracted as blank. The final result was the average of two measurements, each over 10 seconds. The 10 mg/mL sample solution was prepared using MeOH as solvent.

GCMS analysis is performed on a Hewlett Packard instrument (HP6890 Series gas chromatograph with a Mass Selective Detector 5973; injector volume: 1 mL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.) or Varian 400 MHz MR NMR (Palo Alto, Calif.). The spectral reference was either TMS or the known chemical shift of the solvent. Some compound samples were run at elevated temperatures (e.g., 75° C.) to promote increased sample solubility. Melting points are determined on a Laboratory Devices MeI-Temp apparatus (Holliston, Mass.).

Preparative separations are carried out using a Combiflash Rf system (Teledyne Isco, Lincoln, Nebr.) with RediSep silica gel cartridges (Teledyne Isco, Lincoln, Nebr.) or SiliaSep silica gel cartridges (Silicycle Inc., Quebec City, Canada) or by flash column chromatography using silica gel (230-400 mesh) packing material, or by HPLC using a Waters 2767 Sample Manager, C-18 reversed phase column, 30×50 mm, flow 75 mL/min. Typical solvents employed for the Combiflash Rf system and flash column chromatography are dichloromethane, methanol, ethyl acetate, hexane, heptane, acetone, aqueous ammonia (or ammonium hydroxide), and triethyl amine. Typical solvents employed for the reverse phase HPLC are varying concentrations of acetonitrile and water with 0.1% trifluoroacetic acid.

The following abbreviations have the following meanings. If not specifically defined, abbreviations will have their generally accepted meanings.

### ABBREVIATIONS

### ACN: Acetonitrile

BINAP: 2,2′-bis(diphenylphosphino)-1,1′-binapthyl
BOC-anhydride: di-tert-butyl dicarbonate
bp: boiling point
d: days
DAST: Diethylaminosulfur trifluoride
DBU: 1,8-Diazabicyclo[5.4.0]undec-7-ene

### DCM: Dichloromethane

DIEA: diisopropylethylamine

### DIPEA: N,N-diisopropylethylamine

### DMAP: 4-Dimethylaminopyridine

DME: 1,2-dimethoxyethane

### DMF: N,N-dimethylformamide

DMSO: dimethyl sulfoxide
dppf: 1,1′-bis(diphenylphosphino)ferrocene
eq: equivalent
EtOAc: ethyl acetate
EtOH: ethanol
GCMS: gas chromatography-mass spectrometry
HATU: 2-(7-aza-1H-benzotriazole-1-yl)-1,1,3,3-tetramethyluronium hexafluorophosphate
HPLC or hplc: high performance liquid chromatography
hr: hour
hrs: hours
KO-tBu: potassium tert-butoxide
LHMDS: Lithium bis(trimethylsilyl)amide
MCPBA: meta-chloroperoxybenzoic acid
MeOH: methanol
n.a.: not available
NaH: sodium hydride

### NBS: N-bromosuccinimide

NEt₃: triethylamine
NMP: N-methyl-2-pyrrolidone
Rt: retention time
THF: tetrahydrofuran
TLC: thin layer chromatography

Compounds of the present invention can be synthesized by procedures known to one skilled in the art and the general schemes outlined below.

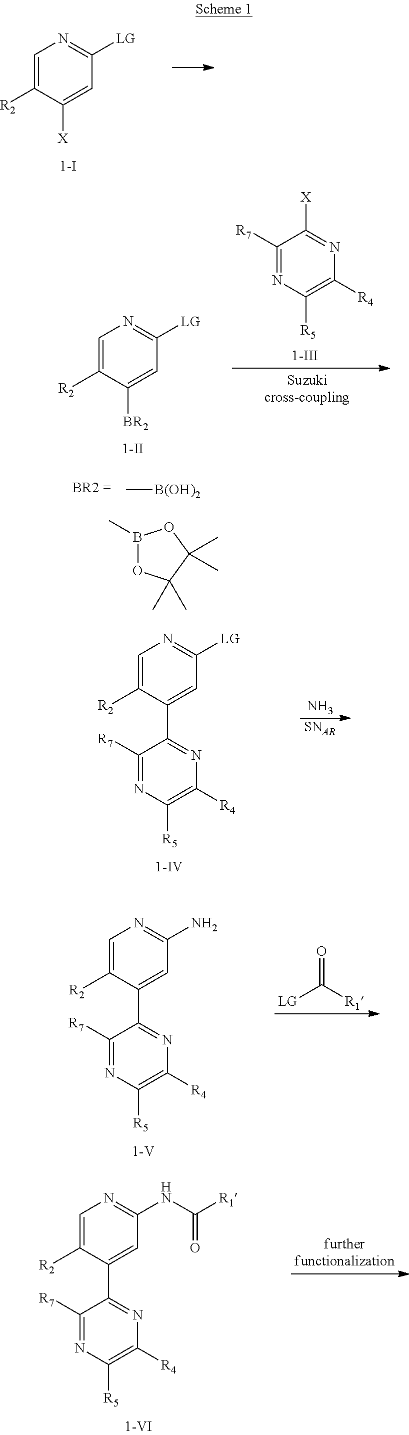

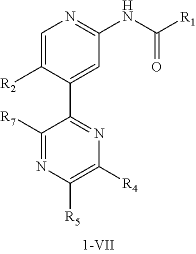

As shown in Scheme 1, synthesis can start with a functionalized pyridine I wherein LG is a leaving group such as F, Cl, OTf, and the like. X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and pyrazine III then gives bi-heteroaryl intermediate IV. The SN_{AR} reaction between IV and ammonium hydroxide in a solvent such as DMF, THF, DMSO, NMP, dioxane with heating (30-130° C.) can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional munipulation is needed to obtain VII. When R₁′ is identical to R₁, compound VII will be the same as compound VI.

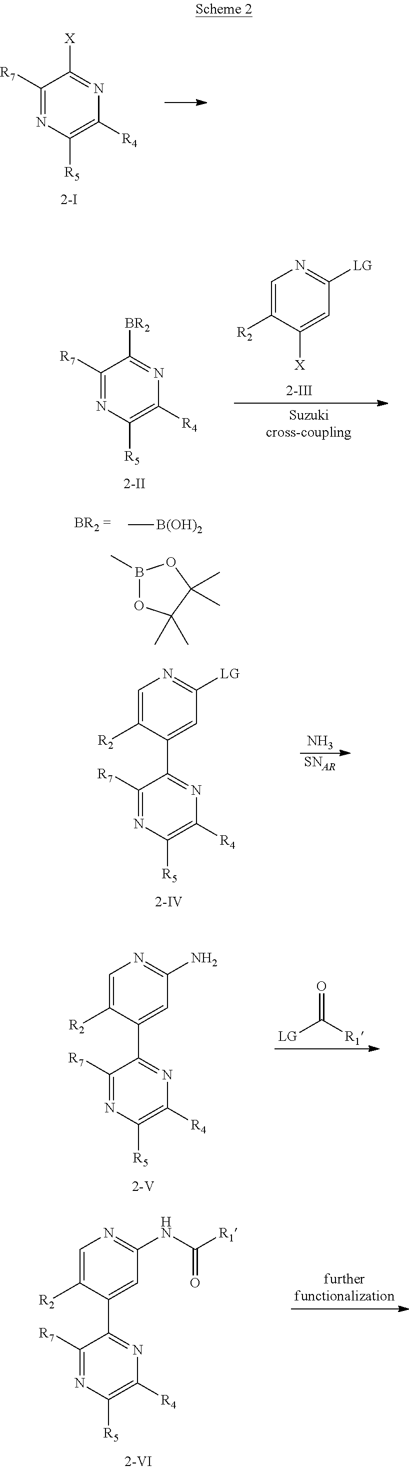

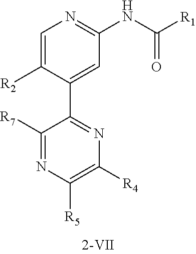

Another alternative route is illustrated in Scheme 2. Synthesis can start with a functionalized pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional munipulation is needed to obtain VII. When R₁′ is identical to R₁, compound VII will be the same as compound VI.

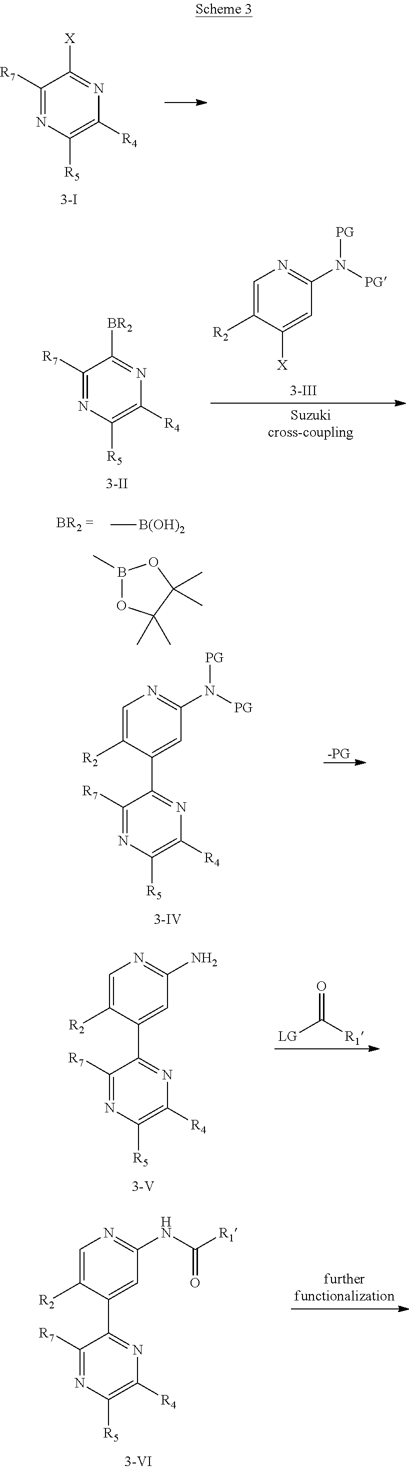

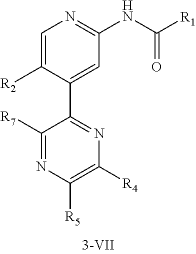

Another alternative route is illustrated in Scheme 3. Synthesis can start with a functionalized pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. Removal of protecting groups PG can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional munipulation is needed to obtain VII. When R₁′ is identical to R₁, compound VII will be the same as compound VI.

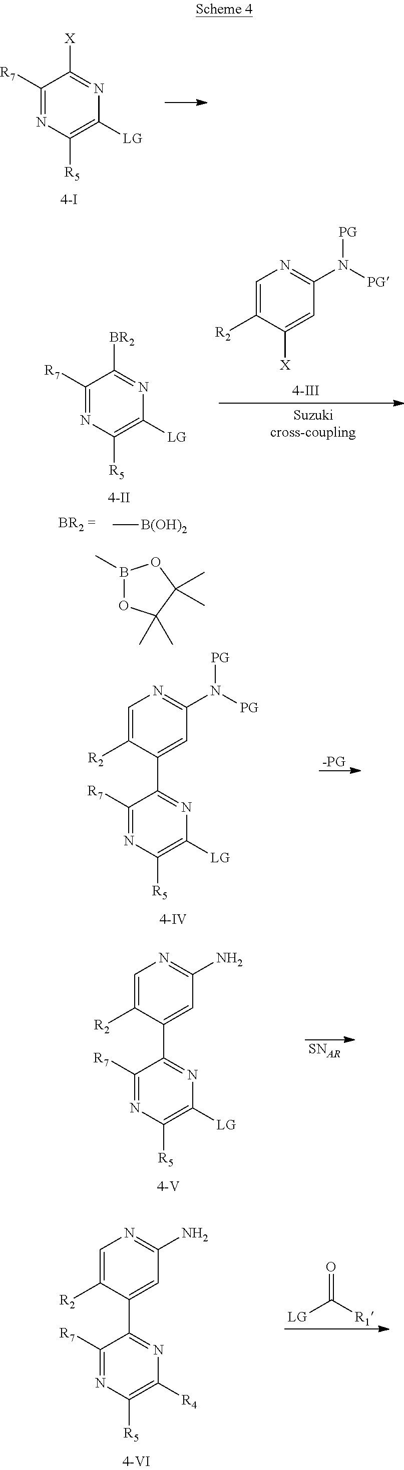

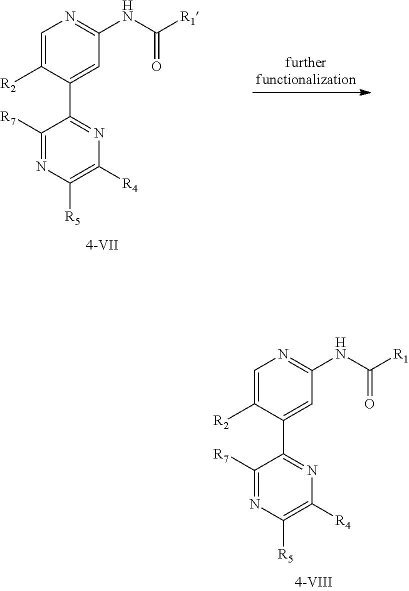

Another alternative route is illustrated in Scheme 4. Synthesis can start with a functionalized pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. Removal of protecting groups PG can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional munipulation is needed to obtain VIII. When R₁′ is identical to R₁, compound VIII will be the same as compound VII.

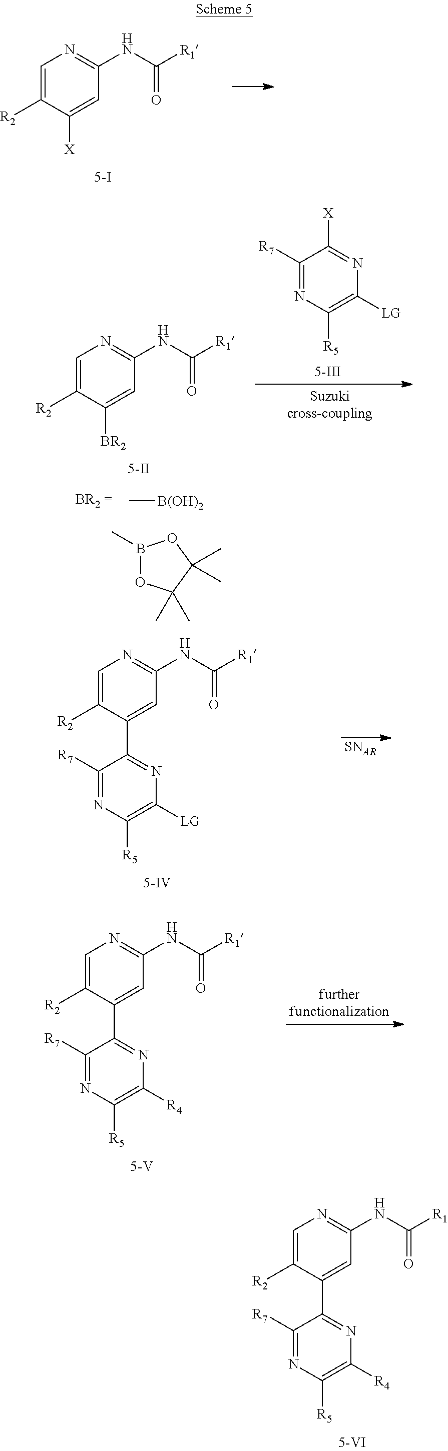

Another alternative route is illustrated in Scheme 5. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyrazine III then gives bi-heteroaryl intermediate IV. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound V. When R₁′ is not identical to R₁, further functional munipulation is needed to obtain VI. When R₁′ is identical to R₁, compound VI will be the same as compound V.

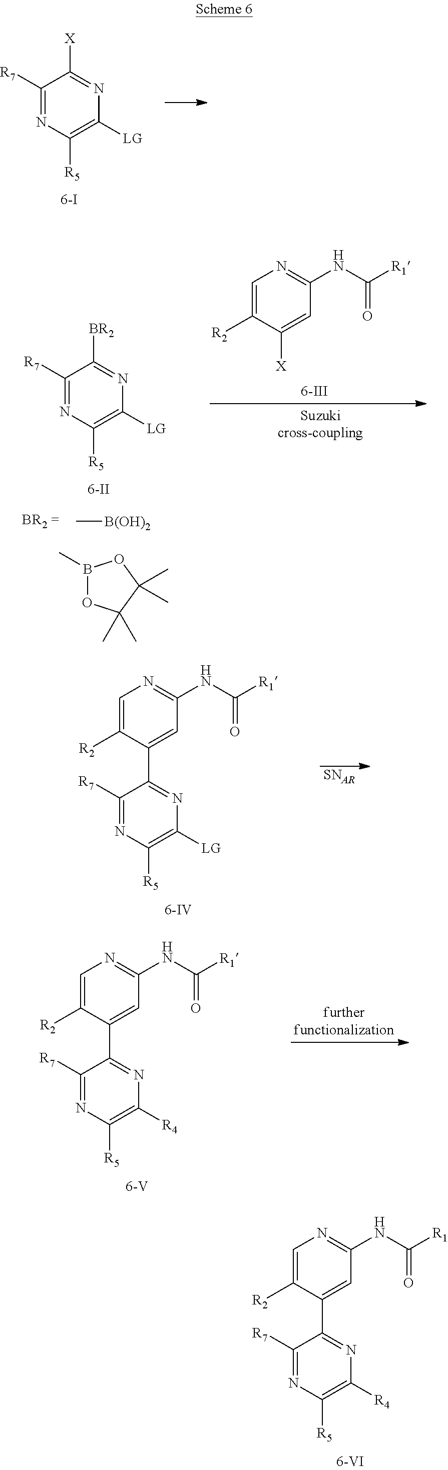

Another alternative route is illustrated in Scheme 6. Synthesis can start with a functionalized pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound V. When R₁′ is not identical to R₁, further functional munipulation is needed to obtain VI. When R₁′ is identical to R₁, compound VI will be the same as compound V.

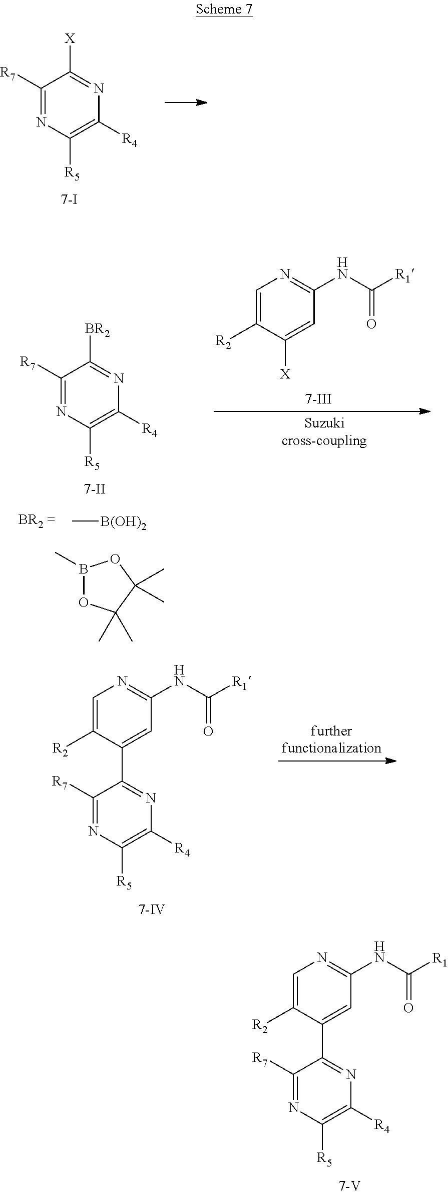

Another alternative route is illustrated in Scheme 7. Synthesis can start with a functionalized pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. When R₁′ is not identical to R₁, further functional munipulation is needed to obtain VI. When R₁′ is identical to R₁, compound VI will be the same as compound V.

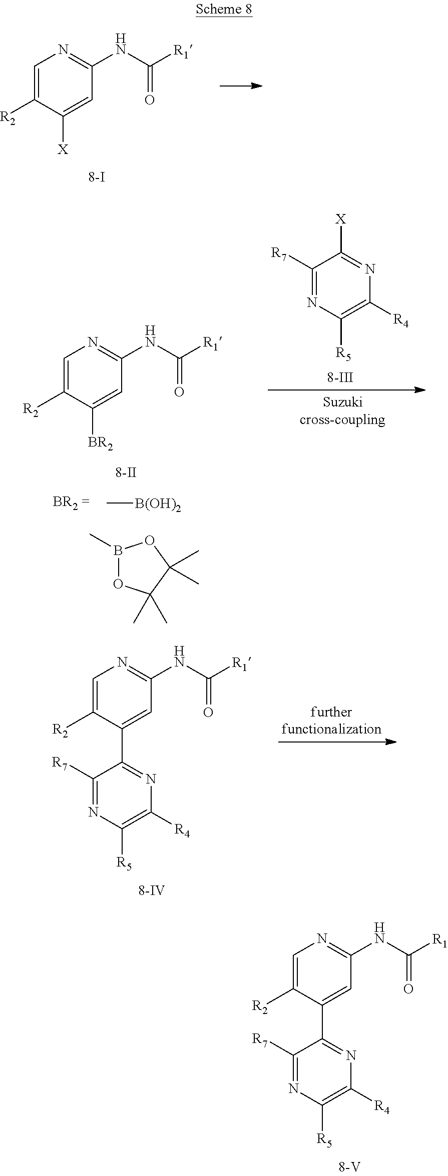

Another alternative route is illustrated in Scheme 8. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyrazine III then gives bi-heteroaryl intermediate IV. When R₁′ is not identical to R₁, further functional munipulation is needed to obtain VI. When R₁′ is identical to R₁, compound VI will be the same as compound V.

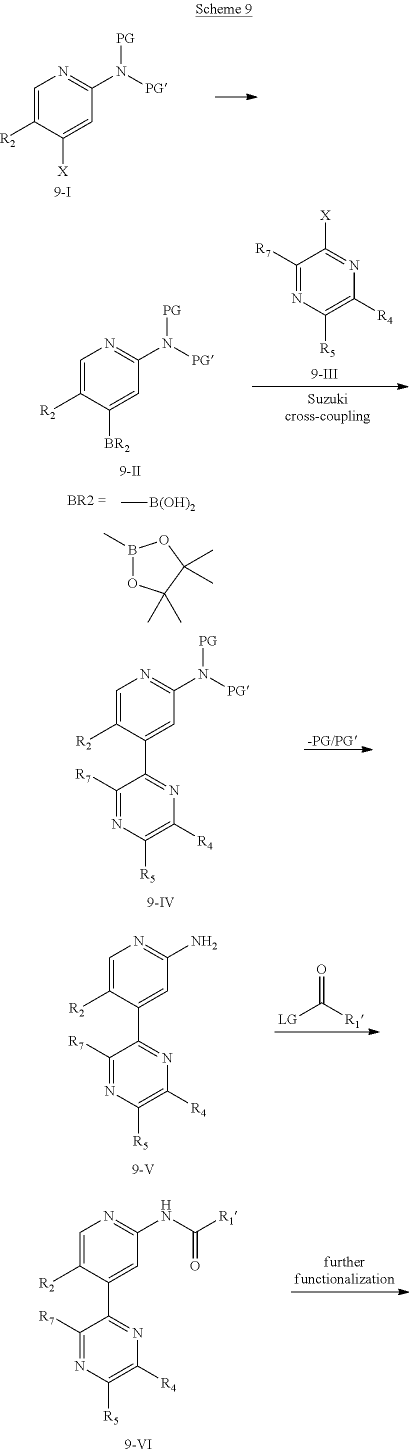

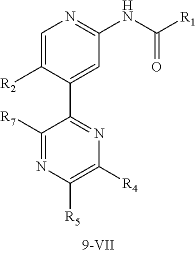

Another alternative route is illustrated in Scheme 9. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyrazine III then gives bi-heteroaryl intermediate IV. Removal of protecting groups PG can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional munipulation is needed to obtain VII. When R₁′ is identical to R₁, compound VII will be the same as compound VI.

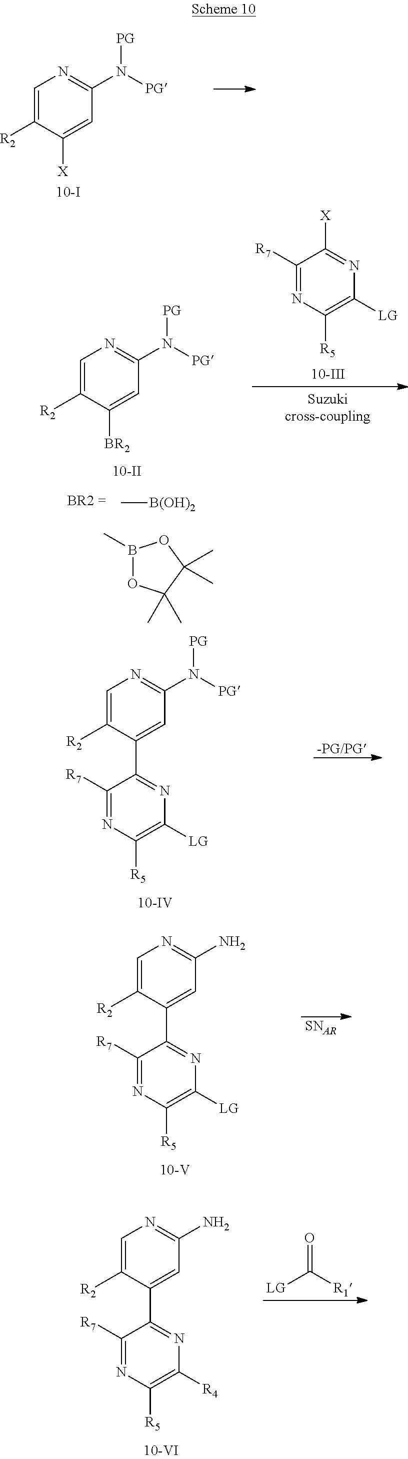

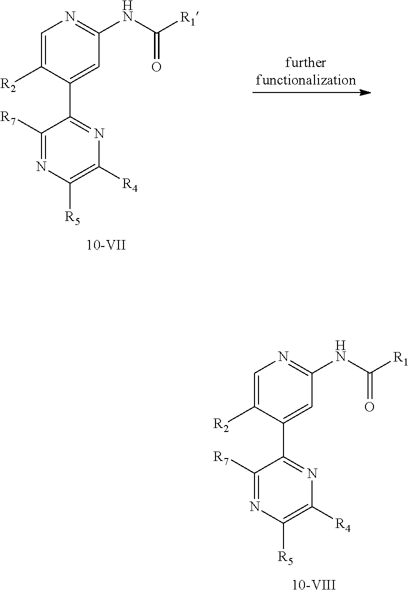

Another alternative route is illustrated in Scheme 10. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyrazine III then gives bi-heteroaryl intermediate IV. Removal of protecting groups PG can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional munipulation is needed to obtain VIII. When R₁′ is identical to R₁, compound VIII will be the same as compound VII.

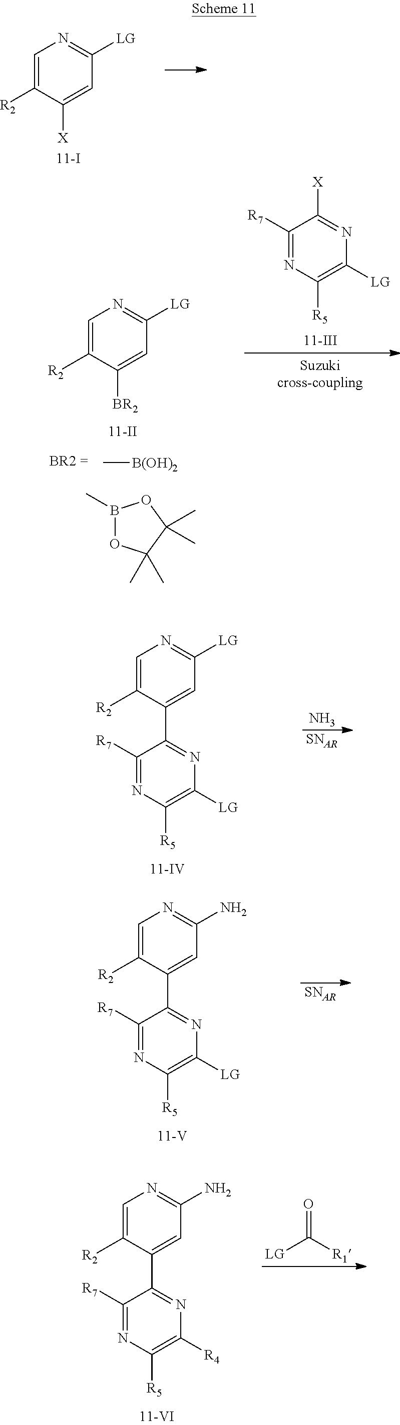

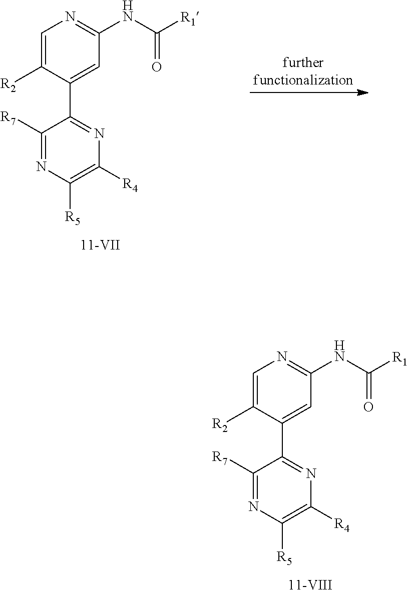

Another alternative route is illustrated in Scheme 11. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyrazine III then gives bi-heteroaryl intermediate IV. The SN_{AR} reaction between IV and ammonium hydroxide in a solvent such as DMF, THF, DMSO, NMP, dioxane with heating (30-130° C.) can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional munipulation is needed to obtain VIII. When R₁′ is identical to R₁, compound VIII will be the same as compound VII.

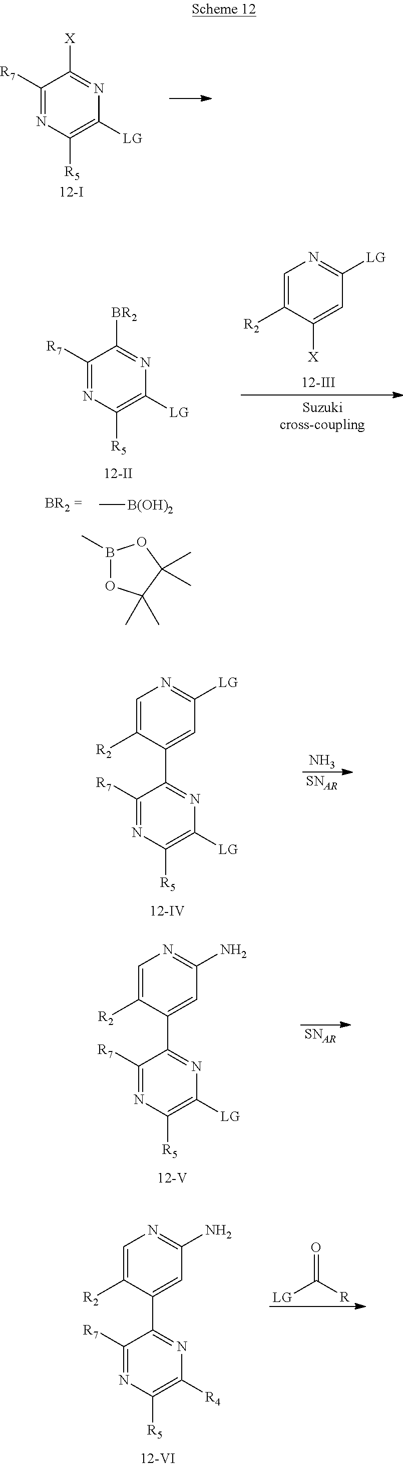

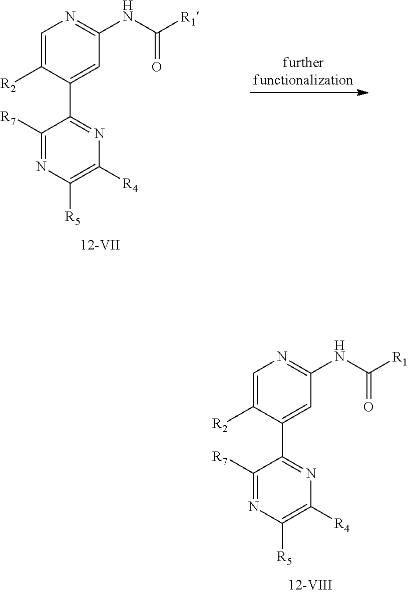

Another alternative route is illustrated in Scheme 12. Synthesis can start with a functionalized pyridine or pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and functionalize pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional munipulation is needed to obtain VIII. When R₁′ is identical to R₁, compound VIII will be the same as compound VII.

### Synthesis of Intermediates

### Synthesis of 6-chloro-N-((tetrahydro-2H-pyran-4-yl)methyl)pyrazin-2-amine

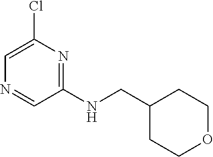

To a solution of 2,6-dichloropyrazine (950 mg, 6.38 mmol) in DMSO (14 mL) was added triethylamine (1.067 mL, 7.65 mmol) and (tetrahydro-2H-pyran-4-yl)methanamine (771 mg, 6.70 mmol). The mixture was heated at 75° C. for 6 hrs. The mixture was allowed to cool to room temperature and diluted with EtOAc (300 mL). The organic layer was washed with 1N aqueous sodium hydroxide solution (1×), water (3×), and brine (1×), dried over sodium sulfate, filtered off and concentrated under reduced pressure providing crude 6-chloro-N-((tetrahydro-2H-pyran-4-yl)methyl)pyrazin-2-amine (1185 mg), which was directly used in the next step without further purification. LCMS (m/z): 228.0 [M+H]+; Rt=0.73 min.

### Alternative preparation of 6-chloro-N-((tetrahydro-2H-pyran-4-yl)methyl)pyrazin-2-amine

To a solution of 2,6-dichloropyrazine (300 mg, 2.014 mmol) in DMSO (3 mL) was added (tetrahydro-2H-pyran-4-yl)methanamine hydrochloride (366 mg, 2.416 mmol) and triethylamine (0.674 mL, 4.83 mmol). The mixture was heated at 90° C. for 4 hrs. The mixture was allowed to cool to room temperature and diluted with EtOAc. The organic layer was washed with 3N aqueous sodium hydroxide solution (1×), water (3×), and brine (1×), dried over sodium sulfate, filtered off and concentrated under reduced pressure providing crude 6-chloro-N-((tetrahydro-2H-pyran-4-yl)methyl)pyrazin-2-amine (400 mg), which was directly used without further purification. LCMS (m/z): 228.0/230.0 [M+H]+; Rt=0.69 min.

### Synthesis of 6-(2-amino-5-chloropyridin-4-yl)-N-((tetrahydro-2H-pyran-4-yl)methyl)pyrazin-2-amine

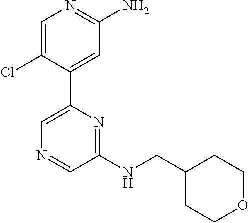

### Step 1: Preparation of 6-(5-chloro-2-fluoropyridin-4-yl)-N-((tetrahydro-2H-pyran-4-yl)methyl)pyrazin-2-amine

A mixture of 6-chloro-N-((tetrahydro-2H-pyran-4-yl)methyl)pyrazin-2-amine (272 mg, 1 mmol), 5-chloro-2-fluoropyridin-4-ylboronic acid (351 mg, 2.000 mmol), PdCl₂(dppf) CH₂Cl₂ adduct (82 mg, 0.100 mmol) in DME (4.5 mL) and 2M aqueous sodium carbonate solution (1.5 mL) in a sealed tube was heated at 103° C. for 2 hrs. The mixture was cooled to room temperature and was diluted with EtOAc (˜25 mL) and MeOH (˜5 mL), filtered off and concentrated under reduced pressure. The residue was purified by column chromatography [silica gel, 40 g, EtOAc/heptane=50/50 to 80/20]. Fractions were combined and concentrated under reduced pressure providing 6-(5-chloro-2-fluoropyridin-4-yl)-N-((tetrahydro-2H-pyran-4-yl)methyl)pyrazin-2-amine (54 mg). LCMS (m/z): 323.0/324.9 [M+H]+; Rt=0.82 min.

### Step 2: Preparation of 6-(2-amino-5-chloropyridin-4-yl)-N-((tetrahydro-2H-pyran-4-yl)methyl)pyrazin-2-amine

A mixture of 6-(5-chloro-2-fluoropyridin-4-yl)-N-((tetrahydro-2H-pyran-4-yl)methyl)pyrazin-2-amine (50 mg, 0.155 mmol) and aqueous ammonium hydroxide solution (30-35 wt. %, 1.5 mL) in DMSO (1.8 mL) and under argon was heated in a microwave reactor at 125° C. for 210 min. The mixture was diluted with EtOAc and brine. The separated organic layer was washed with water and brine, dried over sodium sulfate, filtered off and concentrated under reduced pressure providing crude 6-(2-amino-5-chloropyridin-4-yl)-N-((tetrahydro-2H-pyran-4-yl)methyl)pyrazin-2-amine (41 mg), which was directly used in the next step without further purification. LCMS (m/z): 318.9/320.8 [M+H]+; Rt=0.48 min.

### Alternative preparation of 6-(2-amino-5-chloropyridin-4-yl)-N-((tetrahydro-2H-pyran-4-yl)methyl)pyrazin-2-amine

### Step 1: Preparation of 6-(5-chloro-2-fluoropyridin-4-yl)-N-((tetrahydro-2H-pyran-4-yl)methyl)pyrazin-2-amine

A mixture of 6-chloro-N-((tetrahydro-2H-pyran-4-yl)methyl)pyrazin-2-amine (1620 mg, 7.11 mmol), 5-chloro-2-fluoropyridin-4-ylboronic acid (2246 mg, 12.81 mmol) and PdCl₂(dppf) CH₂Cl₂ adduct (465 mg, 0.569 mmol) in DME (25 mL) and 2M aqueous sodium carbonate solution (10.67 mL) was heated in a sealed tube at 110-115° C. for 90 min. The mixture was cooled to room temperature and was diluted with EtOAc (˜30 mL) and MeOH (˜20 mL), filtered off and concentrated under reduced pressure. The residue was purified by column chromatography [silica gel, 120 g, EtOAc/heptane=20/80 to 75/25]. Fractions were combined and concentrated under reduced pressure providing 6-(5-chloro-2-fluoropyridin-4-yl)-N-((tetrahydro-2H-pyran-4-yl)methyl)pyrazin-2-amine (891 mg). LCMS (m/z): 323.0 [M+H]+; Rt=0.92 min.

### Step 2: Preparation of 6-(2-amino-5-chloropyridin-4-yl)-N-((tetrahydro-2H-pyran-4-yl)methyl)pyrazin-2-amine

A mixture of 6-(5-chloro-2-fluoropyridin-4-yl)-N-((tetrahydro-2H-pyran-4-yl)methyl)pyrazin-2-amine (1090 mg, 3.38 mmol) and aqueous aqueous ammonium hydroxide solution (30-35 wt. %, 25 mL) in DMSO (25 mL) was heated in a sealed steel bomb at 135-140° C. for 18 hrs. The mixture was cooled to room temperature and diluted with EtOAc (300 mL), washed with water (3×), brine (1×), dried sodium sulfate, filtered off and concentrate under reduced pressure. The residue was purified by column chromatography [silica gel, 24 g, EtOAc/heptane=75/25 to 100/0]. Fractions were combined and concentrated under reduced pressure providing 6-(2-amino-5-chloropyridin-4-yl)-N-((tetrahydro-2H-pyran-4-yl)methyl)pyrazin-2-amine (930 mg). LCMS (m/z): 320.1 [M+H]+; Rt=0.47 min.

### Synthesis of 6-(2-amino-5-chloropyridin-4-yl)-5-methyl-N-((tetrahydro-2H-pyran-4-yl)methyl)pyrazin-2-amine

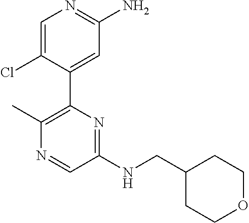

### Step 1: Preparation of 6-(5-chloro-2-fluoropyridin-4-yl)-N-((tetrahydro-2H-pyran-4-yl)methyl)pyrazin-2-amine

6-Chloro-N-((tetrahydro-2H-pyran-4-yl)methyl)pyrazin-2-amine (3.2 g, 14.05 mmol), 5-chloro-2-fluoropyridin-4-ylboronic acid (4.19 g, 23.9 mmol) and 2M aqueous sodium carbonate (16 mL, 0.032 mmol) were dissolved in DME (47 mL). The reaction was then sparged with argon for 3 min, PdCl₂(dppf) CH₂Cl₂ adduct (1.15 g, 1.4 mmol) was added and the mixture was then sparged with argon for additional 2 min. The reaction mixture was heated in a sealed tube at 110° C. for 2 hrs. Additional 5-chloro-2-fluoropyridin-4-ylboronic acid (1.5 g, 8.6 mmol) and PdCl₂(dppf) CH₂Cl₂ adduct (0.620 g, 0.76 mmol) were added and the reaction was stirred at 110° C. for 1 hr. The reaction mixture was cooled to room temperature, diluted with EtOAc (60 mL) and MeOH (20 mL) and filtered through a pad of celites. The filtrate was concentrated under reduced pressure and the residue purified by column chromatography [silica gel, EtOAc/heptane=20/80 to 75/25]. Fractions were combined and concentrated under reduced pressure providing 6-(5-chloro-2-fluoropyridin-4-yl)-N-((tetrahydro-2H-pyran-4-yl)methyl)pyrazin-2-amine (2.7 g). LCMS (m/z): 251.1 [M+H]+; Rt=1.07 min.

### Step 2: Preparation of 5-bromo-6-(5-chloro-2-fluoropyridin-4-yl)-N-((tetrahydro-2H-pyran-4-yl)methyl)pyrazin-2-amine

6-(5-Chloro-2-fluoropyridin-4-yl)-N-((tetrahydro-2H-pyran-4-yl)methyl)pyrazin-2-amine (0.0901 g, 0.279 mmol) was dissolved in a mixture of DMSO (1.13 mL) and water (0.030 mL). N-bromosuccinimide (0.055 g, 0.307 mmol) was added in portions at 0° C. and the resulting mixture was stirred at room temperature for 4 hrs. The reaction mixture was diluted with EtOAc (25 mL) and the organic layer was washed with brine (2×25 mL) and dried over sodium sulfate, filtered off and concentrated under reduced pressure. The residue was purified by column chromatography [silica gel, EtOAc/heptane=25/75 to 100/0]. Fractions were combined and concentrated under reduced pressure providing 5-bromo-6-(5-chloro-2-fluoropyridin-4-yl)-N-((tetrahydro-2H-pyran-4-yl)methyl)pyrazin-2-amine (84.6 mg). LCMS (m/z): 403.0 [M+H]+; Rt=0.95 min. ¹H NMR (400 MHz, DMSO-d6) δ [ppm]: 1.09-1.27 (m, 2H) 1.58 (d, J=12.91 Hz, 2H) 1.76 (ddd, J=10.96, 7.04, 3.91 Hz, 1H) 3.09 (t, J=6.06 Hz, 2H) 3.23 (t, J=10.96 Hz, 3H) 3.82 (dd, J=11.35, 2.74 Hz, 2H) 7.52 (d, J=2.74 Hz, 1H) 7.64 (t, J=5.28 Hz, 1H) 7.88 (s, 1H) 8.50 (s, 1H).

### Step 3: Preparation of 6-(5-chloro-2-fluoropyridin-4-yl)-5-methyl-N-((tetrahydro-2H-pyran-4-yl)methyl)pyrazin-2-amine

5-Bromo-6-(5-chloro-2-fluoropyridin-4-yl)-N-((tetrahydro-2H-pyran-4-yl)methyl)pyrazin-2-amine (0.814 g, 0.203 mmol), potassium methyltrifluoroborate (0.037 g, 0.304 mmol), and potassium phosphate (0.258 g, 1.22 mmol) were dissolved in a mixture of toluene (2.3 mL) and water (0.39 mL). The solution was then degassed by sparging with argon for 5 min and PdCl₂(dppf) CH₂Cl₂ adduct (0.033 g, 0.041 mmol) was added. The mixture was heated in the microwave reactor at 115° C. for 25 min. Additional potassium methyltrifluoroborate (0.074 g, 0.608 mmol) and PdCl₂(dppf) CH₂Cl₂ adduct (0.033 g, 0.041 mmol) were added and the reaction mixture was heated in the microwave at 115° C. for additional 30 min. The mixture was filtered through a plug of celites and the filtrate was concentrated under reduced pressure. The residue was purified by column chromatography [silica gel, EtOAc/heptane=25/75 to 100/0]. Fractions were combined and concentrated under reduced pressure providing 6-(5-chloro-2-fluoropyridin-4-yl)-5-methyl-N-((tetrahydro-2H-pyran-4-yl)methyl)pyrazin-2-amine (0.0258 g) of. LCMS (m/z): 337.0 [M+H]+; Rt=0.81 min. ¹H NMR (400 MHz, chloroform-d) δ [ppm]: 1.38 (2H) 1.68 (d, J=10.96 Hz, 3H) 1.77-1.94 (m, 1H) 2.17 (s, 1H) 2.26 (s, 4H) 3.26 (t, J=6.46 Hz, 3H) 3.38 (t, J=11.15 Hz, 3H) 3.99 (dd, J=11.35, 3.52 Hz, 2H) 4.64-4.73 (m, 1H) 6.94 (d, J=2.74 Hz, 1H) 7.27 (s, 1H) 7.92 (s, 1H) 8.31 (s, 1H).

### Step 4: Preparation of 6-(2-amino-5-chloropyridin-4-yl)-5-methyl-N-((tetrahydro-2H-pyran-4-yl)methyl)pyrazin-2-amine

A mixture of 6-(5-chloro-2-fluoropyridin-4-yl)-5-methyl-N-((tetrahydro-2H-pyran-4-yl)methyl)pyrazin-2-amine (0.0338 g, 0.100 mmol) and aqueous ammonium hydroxide solution (30-35 wt. %, 3 mL) in DMSO (3 mL) was heated in a steel bomb at 135° C. for 18 hrs. The reaction mixture was diluted with water (25 mL) and extracted with EtOAc (3×50 mL). The combined organic extracts were washed with brine (1×25 mL), dried over sodium sulfate, filtered off and concentrated under reduced pressure providing 6-(2-amino-5-chloropyridin-4-yl)-5-methyl-N-((tetrahydro-2H-pyran-4-yl)methyl)pyrazin-2-amine (0.0369 g), which was directly used without further purification. LCMS (m/z): 334.1 [M+H]+; Rt=0.49 min.

### Synthesis of (R)-tert-butyl 3-(5-chloro-4-(6-chloropyrazin-2-yl)pyridin-2-ylcarbamoyl)piperidine-1-carboxylate

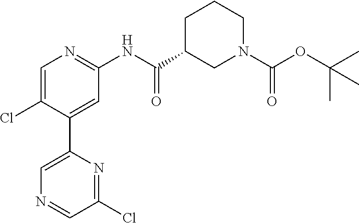

### Step 1: Preparation of 2-chloro-6-(4,4,5,5-tetramethyl-1,3,2-dioxaborolan-2-yl)pyrazine

A mixture of tris(dibenzylideneacetone)dipalladium(0) (0.275 g, 0.300 mmol) and tricyclohexylphosphine (0.202 g, 0.720 mmol) in dioxane (50 mL) under argon was stirred for 30 min at room temperature. 2,6-Dichloropyrazine, bis(pinacolato)diboron (2.79 g, 11.00 mmol) and potassium acetate (1.472 g, 15.00 mmol) were added and the mixture was stirred at 80° C. for 18 hrs. The mixture was cooled to room temperature and filtered through a thin layer of celites. The filtrate was concentrated under reduced pressure and providing crude 2-chloro-6-(4,4,5,5-tetramethyl-1,3,2-dioxaborolan-2-yl)pyrazine, which was directly used in the next step without purification. LCMS (m/z): 158.9 [boronic acid fragment+H]+; Rt=0.33 min.

### Step 2: Preparation of (R)-tert-butyl 3-(5-chloro-4-(6-chloropyrazin-2-yl)pyridin-2-ylcarbamoyl)piperidine-1-carboxylate

A mixture of 2-chloro-6-(4,4,5,5-tetramethyl-1,3,2-dioxaborolan-2-yl)pyrazine (1.162 g, 4.83 mmol) and (R)-tert-butyl 3-(5-chloro-4-iodopyridin-2-ylcarbamoyl)piperidine-1-carboxylate (1.5 g, 3.22 mmol) and PdCl₂(dppf) CH₂Cl₂ adduct (0.263 g, 0.322 mmol) in 2M aqueous sodium carbonate solution (4.83 mL) was purged with argon. DME (10 mL) was added and the resulting mixture was stirred for 2 hrs at 90° C. The reaction mixture was allowed to cool to room temperature and diluted with EtOAc (50 mL). The mixture was washed with water and brine, dried over magnesium sulfate, filtered off and concentrated under reduced pressure. The residue was purified by column chromatography [silica gel, 40 g, EtOAc/heptane=0/100 to 40/60] providing (R)-tert-butyl 3-(5-chloro-4-(6-chloropyrazin-2-yl)pyridin-2-ylcarbamoyl)piperidine-1-carboxylate (1.1 g). LCMS (m/z): 453.2 [M+H]+; Rt=1.05 min.

### Synthesis of (R)-tert-butyl 3-(5-chloro-4-iodopyridin-2-ylcarbamoyl)piperidine-1-carboxylate

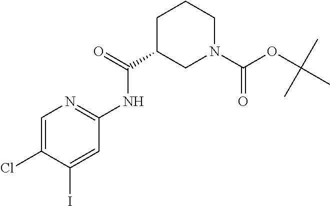

### Step 1: Preparation of 5-chloro-4-iodopyridin-2-amine

A mixture of 5-chloro-2-fluoro-4-iodopyridine (4.120 g, 16.00 mmol) and aqueous ammonium hydroxide solution (32 wt. %, 70 mL) in DMSO (70 mL) was heated in a sealed steel bomb at 90° C. for 18 hrs. The mixture was cooled to room temperature and diluted with EtOAc (450 mL). The mixture was washed with water (3×) and brine (1×), dried over sodium sulfate, filtered off and concentrate under reduced pressure providing crude 5-chloro-4-iodopyridin-2-amine (3.97 g), which was directly used in the next step without further purification. LCMS (m/z): 254.9 [M+H]+; Rt=0.43 min.

### Step 2: Preparation of (R)-tert-butyl 3-(5-chloro-4-iodopyridin-2-ylcarbamoyl)piperidine-1-carboxylate

To a solution of (R)-1-(tert-butoxycarbonyl)piperidine-3-carboxylic acid (1.081 g, 4.72 mmol) in dichloromethane (6 mL) at 0° C. was added 1-chloro-N,N,2-trimethylprop-1-en-1-amine (0.735 g, 5.50 mmol). The mixture was stirred at room temperature for 30 min and added to a solution of 5-chloro-4-iodopyridin-2-amine (1.00 g, 3.93 mmol) and pyridine (0.445 mL, 5.50 mmol) in tetrahydrofuran (6 mL). The reaction mixture was stirred at room temperature for 2 hrs. The mixture was diluted with EtOAc (350 mL) and washed with saturated aqueous sodium bicarbonate solution (1×), water (2×), brine (1×), dried over sodium sulfate, filtered off and concentrated under reduced pressure. The residue was purified by column chromatography [silica gel, 40 g, EtOAc/heptane=0/100 to 75/25] providing (R)-tert-butyl 3-(5-chloro-4-iodopyridin-2-ylcarbamoyl)piperidine-1-carboxylate (1.80 g). LCMS (m/z): 466.0 [M+H]+; Rt=1.06 min.

### Synthesis of 2,5-difluoropyridin-4-ylboronic acid

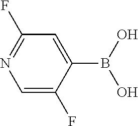

To a solution of diisopropylamine (1.74 mL, 12.20 mmol) in anhydrous tetrahydrofuran (22 mL) under argon at −20° C. was added n-butyllithium (7.66 mL, 1.6M in hexanes) slowly over 10 min. The newly formed LDA was then cooled to −78° C. A solution of 2,5-difluoropyridine (1.05 mL, 11.5 mmol) in anhydrous tetrahydrofuran (3 mL) was added slowly over 30 min and the mixture was stirred at −78° C. for 4 hrs. A solution of triisopropyl borate (5.90 mL, 25.4 mmol) in anhydrous tetrahydrofuran (8.6 mL) was added dropwise. Once the addition was complete the reaction mixture was warmed to room temperature and stirring was continued for an additional hour. The reaction mixture was diluted with aqueous sodium hydroxide solution (4 wt. %, 34 mL). The separated aqueous layer was cooled to 0° C. and then slowly acidified to pH=4 with 6N aqueous hydrochloride solution (˜10 mL). The mixture was extracted with EtOAc (3×50 mL). The combined organic layers washed with brine (50 mL), dried over sodium sulfate, filtered off and concentrated under reduced pressure. The residue was triturated with diethylether to give 2,5-difluoropyridin-4-ylboronic acid (808 mg).

### Synthesis of (S)-1-(tetrahydro-2H-pyran-4-yl)ethanamine

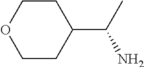

### Step 1: Preparation of (R,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (R)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (R,E)-2-methyl-N-((tetrahydro-2H-pyran-4-yl)methylene)propane-2-sulfinamide (1.9 g). LCMS (m/z): 218.1 [M+H]+; Rt=0.58 min.

### Step 2: Preparation of (R)-2-methyl-N—((S)-1-(tetrahydro-2H-pyran-4-yl)ethyl)propane-2-sulfinamide

To a solution of (R,E)-2-methyl-N-((tetrahydro-2H-pyran-4-yl)methylene)propane-2-sulfinamide (0.93 g, 4.28 mmol) in dichloromethane (21.4 mL) at 0° C. was added slowly methylmagnesium bromide (2.0 M in tetrahydrofuran, 4.28 mL, 8.56 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatography providing (R)-2-methyl-N—((S)-1-(tetrahydro-2H-pyran-4-yl)ethyl)propane-2-sulfinamide (910 mg). LCMS (m/z): 234.0 [M+H]+; Rt=0.58 min.

### Step 3: Preparation of (S)-1-(tetrahydro-2H-pyran-4-yl)ethanamine

To a solution of (R)-2-methyl-N—((S)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (S)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane. The organic layer was dried over sodium sulfate, filtered off and concentrated under reduced pressure providing crude (S)-1-(tetrahydro-2H-pyran-4-yl)ethanamine (212 mg), which was directly used in the next reaction without further purification. LCMS (m/z): 130.1 [M+H]+; Rt=0.34 min.

### Synthesis of (R)-1-(tetrahydro-2H-pyran-4-yl)ethanamine

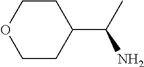

### Step 1: Preparation of (S,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (S)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (S,E)-2-methyl-N-((tetrahydro-2H-pyran-4-yl)methylene)propane-2-sulfinamide (1.50 g). LCMS (m/z): 218.1 [M+H]+; Rt=0.58 min.

### Step 2: Preparation of (S)-2-methyl-N—((R)-1-(tetrahydro-2H-pyran-4-yl)ethyl)propane-2-sulfinamide

To a solution of (S,E)-2-methyl-N-((tetrahydro-2H-pyran-4-yl)methylene)propane-2-sulfinamide (1.5 g, 6.90 mmol) in dichloromethane (34.5 mL) at 0° C. was slowly added methylmagnesium bromide (1.646 g, 13.80 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatograph providing (S)-2-methyl-N—((R)-1-(tetrahydro-2H-pyran-4-yl)ethyl)propane-2-sulfinamide (1.40 g). LCMS (m/z): 234.3 [M+H]+; Rt=0.57 min.

### Step 3: Preparation of (R)-1-(tetrahydro-2H-pyran-4-yl)ethanamine

To a solution of (S)-2-methyl-N—((R)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (R)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane (2×). The combined organic layers were dried over sodium sulfate, filtered off and concentrated under reduced pressure providing crude (R)-1-(tetrahydro-2H-pyran-4-yl)ethanamine (200 mg), which was directly used in the next reaction without further purification. LCMS (m/z): 130.1 [M+H]+; Rt=0.34 min.

### Synthesis of (2,2-dimethyltetrahydro-2H-pyran-4-yl)methanamine

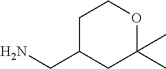

### Step 1: Preparation of (2,2-dimethyltetrahydro-2H-pyran-4-yl)methyl 4-methylbenzenesulfonate

To a solution of (2,2-dimethyltetrahydro-2H-pyran-4-yl)methanol (1 g, 6.93 mmol) in dichloromethane (5 mL) and pyridine (5 mL, 61.8 mmol) was added para-toluenesulfonyl chloride (1.586 g, 8.32 mmol) and DMAP (0.042 g, 0.347 mmol). The resulting mixture was stirred for 18 hrs at room temperature. The reaction mixture was concentrated under reduced pressure and the residue was diluted with water and dichloromethane. The separated organic phase was washed with 0.2N aqueous hydrochloride solution (1×), 1N aqueous hydrochloride solution (2×), brine, dried over sodium sulfate, filtered off and concentrated under reduced pressure. The residue was purified by column chromatography [silica gel, 40 g, EtOAc/hexane=0/100 to 50/50] providing (2,2-dimethyltetrahydro-2H-pyran-4-yl)methyl 4-methylbenzenesulfonate (2.05 g) as a colorless oil. LCMS (m/z): 299.1 [M+H]+; Rt=0.96 min.

### Step 2: Preparation of (2,2-dimethyltetrahydro-2H-pyran-4-yl)methanamine

Into a solution of (2,2-dimethyltetrahydro-2H-pyran-4-yl)methyl 4-methylbenzenesulfonate (3 g, 10.05 mmol) in tetrahydrofuran (25 mL) in a steel bomb was condensed ammonia (˜5.00 mL) at −78° C. The mixture was heated in the steel bomb at 125° C. for ˜18 hrs. The mixture was cooled to −78° C., the steel bomb was opened, and the mixture was allowed to warm up to room temperature under a stream of nitrogen. The mixture was concentrated under reduced pressure and the residue was partitioned between a aqueous sodium hydroxide solution (5 wt. %) and dichloromethane. The separated aqueous layer was extracted with dichloromethane (1×). The combined organic layers were washed with aqueous sodium hydroxide solution (5 wt. %), dried over sodium sulfate, filtered off and concentrated under reduced pressure providing crude (2,2-dimethyltetrahydro-2H-pyran-4-yl)methanamine (˜2.36 g) as yellow liquid, which was directly used in the next reaction without further purification. LCMS (m/z): 144.1 [M+H]+; Rt=0.26 min.

### Synthesis of (6,6-dimethyl-1,4-dioxan-2-yl)methanamine

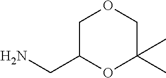

### Step 1: Preparation of 1-(allyloxy)-2-methylpropan-2-ol

To allylic alcohol (57.4 mL, 844 mmol) was added sodium hydride (60 wt. % in mineral oil, 2.43 g, 101 mmol) at 0° C. After stirring for 20 min 2,2-dimethyloxirane (15 mL, 169 mmol) was added and the solution was refluxed overnight. The mixture was allowed to cool to room temperature, diluted with saturated aqueous ammonium chloride solution and extracted with diethylether (3×). The combined organic layers were dried over sodium sulfate, filtered off and concentrated under reduced pressure to remove diethylether. The residue was distilled providing 1-(allyloxy)-2-methylpropan-2-ol (12.3 g, 42 torr, by 58-60° C.) as a colorless oil. ¹H NMR (400 MHz, chloroform-d) δ [ppm]: 5.87-5.96 (m, 1H) 5.26-5.31 (m, 1H) 5.18-5.21 (m, 1H) 4.03-4.05 (m, 2 H) 3.28 (s, 2H) 2.31 (br. s, 1H) 1.23 (s, 3H) 1.22 (s, 3H).

### Step 2: Preparation of 6-(iodomethyl)-2,2-dimethyl-1,4-dioxane

To a solution of 1-(allyloxy)-2-methylpropan-2-ol (5.0 g, 38 mmol) in acetonitrile (400 mL) was added sodium bicarbonate (19.5 g, 77 mmol) and the mixture was cooled to 0° C. Iodine (11.7 g, 46.1 mmol) was added and the reaction mixture was allowed to warm up to room temperature and stirred overnight. To the mixture was added triethylamine (6.42 mL, 46.1 mmol) and additional iodine (7.8 g, 30.7 mmol) and stirring was continued for additional 5 hrs at 0° C. To the mixture was added potassium carbonate (6.37 g, 46.1 mmol) and the suspension was stirred at room temperature for ˜3 days. The reaction mixture was diluted with saturated aqueous sodium thiosulfate solution (200 mL) and EtOAc (300 mL). The separated aqueous layer was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a yellow oil (2.07 g). ¹H NMR (400 MHz, chloroform-d) δ [ppm]: 4.01 (dd, J=11.2, 2.8 Hz, 1H) 3.81-3.88 (m, 1H) 3.44 (d, J=11.2 Hz, 1H) 3.22 (dd, J=11.6, 0.8 Hz, 1H) 2.97-3.13 (m, 3H) 1.33 (s, 3H) 1.14 (s, 3H). 1-(Allyloxy)-2-methylpropan-2-ol (1.63 g) was recovered.

### Step 3: Preparation of 6-(azidomethyl)-2,2-dimethyl-1,4-dioxane

To a solution of 6-(iodomethyl)-2,2-dimethyl-1,4-dioxane (1.80 g, 7.03 mmol) in anhydrous DMF (9 mL) was added sodium azide (0.685 g, 10.5 mmol) and the suspension was heated at 80° C. for 2.5 hrs. The mixture was diluted with water (30 mL) and EtOAc (30 mL). The separated organic layer was washed with water (3×). The aqueous layers were combined and extracted with EtOAc (1×). The combined organic layers, dried over sodium sulfate, filtered off and concentrated under reduced pressure. The residue was purified by column chromatography [silica gel, EtOAc/hexane=10/40 to 20/40] providing 6-(azidomethyl)-2,2-dimethyl-1,4-dioxane (0.93 g) as a colorless oil. ¹H NMR (400 MHz, chloroform-d) δ [ppm]: 4.00-4.06 (m, 1H) 3.75 (ddd, J=11.2, 2.4, 0.4 Hz, 1H) 3.49 (d, J=11.2 Hz, 1H) 3.14-3.29 (m, 4H) 1.35 (s, 3H), 1.14 (s, 3H).

### Step 4: Preparation of (6,6-dimethyl-1,4-dioxan-2-yl)methanamine

To a solution of 6-(azidomethyl)-2,2-dimethyl-1,4-dioxane (502 mg, 2.93 mmol) in anhydrous tetrahydrofuran (15 mL) was added slowly a solution of lithium aluminumhydride (1M in tetrahydrofuran, 3.81 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (6,6-dimethyl-1,4-dioxan-2-yl)methanamine (410 mg) as a colorless oil, which was directly used in the next step without purification. LCMS (m/z): 146.1 [M+H]+; Rt=0.42 min.

### Synthesis of (5,5-dimethyl-1,4-dioxan-2-yl)methanamine

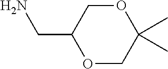

### Step 1: Preparation of 2-(allyloxy)-2-methylpropan-1-ol

To a solution of 2,2-dimethyloxirane (15.0 mL, 169 mmol) in allylic alcohol (57.4 mL) was added perchloric acid (70 wt. %, 7.26 mL, 84 mmol) slowly at 0° C. The solution was warmed to room temperature and stirred for 1.5 hrs. The reaction mixture was diluted with saturated aqueous sodium bicarbonate solution and extracted with diethylether (3×). The combined organic layers were dried over sodium sulfate, filtered off and concentrated under reduced pressure to remove diethylether. The residue was distilled providing 2-(allyloxy)-2-methylpropan-1-ol (9.70 g, 38 torr, by 74-76° C.) as a colorless oil. ¹H NMR (400 MHz, chloroform-d) δ [ppm]: 5.87-5.97 (m, 1H) 5.25-5.31 (m, 1H) 5.12-5.16 (m, 1H) 3.92-3.94 (m, 2H) 3.45 (m, 2H) 1.19 (s, 6H).

### Step 2: Preparation of 5-(iodomethyl)-2,2-dimethyl-1,4-dioxane

To a solution of 2-(allyloxy)-2-methylpropan-1-ol (5.0 g, 38.4 mmol) in acetonitrile (350 mL) was added sodium bicarbonate (9.68 g, 115 mmol) and the mixture was cooled to 0° C. Iodine (29.2 g, 115 mmol) was added and the reaction mixture was allowed to warm up to room temperature and stirred for 6 hrs. The reaction mixture was diluted with saturated aqueous sodium thiosulfate solution and concentrated under reduced pressure removing most of the organic solvent. The residue was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a colorless oil (7.04 g). ¹H NMR (400 MHz, chloroform-d) δ [ppm]: 3.70-3.73 (m, 1H) 3.57-3.64 (m, 2H) 3.43-3.50 (m, 2H) 3.13-3.15 (m, 2 H) 1.32 (s, 3H) 1.13 (s, 3H).

### Step 3: Preparation of 5-(azidomethyl)-2,2-dimethyl-1,4-dioxane

To a solution of 5-(iodomethyl)-2,2-dimethyl-1,4-dioxane (2.58 g, 10.1 mmol) in anhydrous DMF (13 mL) was added sodium azide (0.982 g, 15.1 mmol) and the suspension was heated at 80° C. for 2.5 hrs. The mixture was diluted with water (40 mL) and EtOAc (40 mL). The separated organic layer was washed with water (3×). The aqueous layers were combined and extracted with EtOAc (1×). The combined organic layers, dried over sodium sulfate, filtered off and concentrated under reduced pressure. The residue was purified by column chromatography [silica gel, EtOAc/hexane=10/40 to 50/50] providing 6-(azidomethyl)-2,2-dimethyl-1,4-dioxane (1.61 g) as a colorless oil. NMR (400 MHz, chloroform-d) δ [ppm]: 3.63-3.72 (m, 2H) 3.52-3.59 (m, 2H) 3.42 (d, J=11.6 Hz, 1H) 3.29 (d, J=4.4 Hz, 2H) 1.33 (s, 3H) 1.13 (s, 3H).

### Step 4: Preparation of (5,5-dimethyl-1,4-dioxan-2-yl)methanamine

To a solution of 5-(azidomethyl)-2,2-dimethyl-1,4-dioxane (810 mg, 4.73 mmol) in anhydrous tetrahydrofuran (20 mL) was added slowly a solution of lithium aluminumhydride (1.0 M tetrahydrofuran, 6.2 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (5,5-dimethyl-1,4-dioxan-2-yl)methanamine (673 mg) as a colorless oil, which was directly used in the next step without purification. LCMS (m/z): 146.1 [M+H]+; Rt=0.42 min.

### Synthesis of (4-methyltetrahydro-2H-pyran-4-yl)methanamine

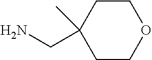

### Step 1: Preparation of 4-methyltetrahydro-2H-pyran-4-carbonitrile

To a solution of tetrahydro-2H-pyran-4-carbonitrile (2 g, 18.00 mmol) in tetrahydrofuran (10 mL) at 0-5° C. was added slowly LHMDS (21.59 mL, 21.59 mmol). The mixture was stirred for 1.5 hrs at 0° C. Iodomethane (3.37 mL, 54.0 mmol) was added slowly and stirring was continued for 30 min at ˜0° C. and then for ˜2 hrs at room temperature. The mixture was cooled to 0° C. and carefully diluted with 1N aqueous hydrochloride solution (30 mL) and EtOAc (5 mL) and concentrated under reduced pressure. The residue was taken up in diethylether and the separated organic layer was washed with brine, dried over sodium sulfate, filtered off and concentrated under reduced pressure providing crude 4-methyltetrahydro-2H-pyran-4-carbonitrile (1.8 g) as an orange oil, which was directly used in the next reaction without further purification. LCMS (m/z): 126.1 [M+H]+; Rt=0.44 min.

### Step 2: Preparation of (4-methyltetrahydro-2H-pyran-4-yl)methanamine

To a solution of 4-methyltetrahydro-2H-pyran-4-carbonitrile (1.8 g, 14.38 mmol) in tetrahydrofuran (30 mL) was carefully added lithium aluminum hydride (1M solution in tetrahydrofuran, 21.57 mL, 21.57 mmol) at 0° C. The reaction mixture was stirred for 15 min at 0° C., allowed to warm to room temperature and stirred for additional 3 hrs at room temperature. To the reaction mixture was carefully added water (0.9 mL) [Caution: gas development!], 1N aqueous sodium hydroxide solution (2.7 mL) and water (0.9 mL). The mixture was vigorously stirred for 30 min. The precipitate was filtered off and rinsed with tetrahydrofuran. The solution was concentrated under reduced pressure providing crude (4-methyltetrahydro-2H-pyran-4-yl)methanamine (1.54 g) as a yellowish solid, which was directly used in the next step without further purification. LCMS (m/z): 130.1 [M+H]+; Rt=0.21 min.

### Synthesis of 4-(aminomethyl)tetrahydro-2H-pyran-4-carbonitrile

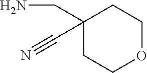

### Step 1: Preparation of dihydro-2H-pyran-4,4(3H)-dicarbonitrile

A mixture of malononitrile (0.991 g, 15 mmol), 1-bromo-2-(2-bromoethoxy)ethane (3.83 g, 16.50 mmol) and DBU (4.97 mL, 33.0 mmol) in DMF (6 mL) was heated at 85° C. for 3 hrs. The reaction mixture was cooled to room temperature and concentrated under reduced pressure. The residue was diluted with EtOAc (25 mL), washed with water (2×10 mL), dried over sodium sulfat, filtered off and concentrated under reduced pressure and further dried in high vacuo providing crude dihydro-2H-pyran-4,4(3H)-dicarbonitrile (1.65 g) as a light brown solid, which was directly used in the next step without further purification. GCMS: 136 [M]; Rt=5.76 min. ¹H NMR (300 MHz, chloroform-d) δ [ppm]: 2.14-2.32 (m, 4H) 3.77-3.96 (m, 4H).

### Step 2: Preparation of 4-(aminomethyl)tetrahydro-2H-pyran-4-carbonitrile

To a solution of dihydro-2H-pyran-4,4(3H)-dicarbonitrile (450 mg, 3.31 mmol in EtOH (15 mL) was added sodium borohydride (375 mg, 9.92 mmol) in portions and the mixture was stirred at room temperature for 4 hrs. The mixture was concentrated under reduced pressure and the residue was diluted with EtOAc (30 mL), washed with water (10 mL), dried over sodium sulfate, filtered off and concentrated under reduced pressure providing crude 4-(aminomethyl)tetrahydro-2H-pyran-4-carbonitrile (388 mg), which was directly used in the next step without further purification. LCMS (m/z): 141.0 [M+H]+; Rt=0.18 min.

### Synthesis of toluene-4-sulfonic acid 4-methoxy-tetrahydro-pyran-4-ylmethyl ester

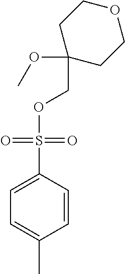

### Step 1: Preparation of 1,6-dioxaspiro[2.5]octane

To a solution of trimethylsulfonium iodide (3.27 g, 16 mmol) in DMSO (20 mL) under nitrogen atmosphere was added dihydro-2H-pyran-4(3H)-one (1.0 g, 10 mmol). To the mixture was added slowly a solution of tert-butoxide (1.68 g, 15 mmol) in DMSO (15 mL) and the solution was stirred at room temperature overnight. The reaction mixture was diluted slowly with water (50 mL) and extracted with diethylether (3×20 mL). The combined organic layers were dried over sodium sulfate, filtered off and concentrated under reduced pressure providing crude 1,6-dioxaspiro[2.5]octane (650 mg), which was directly used without further purification. ¹H NMR (300 MHz, chloroform-d) δ [ppm]: 1.44-1.62 (m, 2H) 1.76-1.98 (m, 2H) 2.70 (s, 2H) 3.70-3.98 (m, 4H).

### Step 2: Preparation of (4-methoxytetrahydro-2H-pyran-4-yl) MeOH

To a solution of 1,6-dioxaspiro[2.5] octane (600 mg, 5.26 mmol) in MeOH (10 mL) under nitrogen was added camphorsulfonic acid (50 mg, 0.21 mmol) at 0° C. and the mixture was stirred at 0° C. for 2 hrs. The mixture was concentrated under reduced pressure providing crude (4-methoxytetrahydro-2H-pyran-4-yl)methanol (707 mg) as a light yellow oil, which was directly used in the next step without further purification. ¹H NMR (300 MHz, chloroform-d) δ [ppm]: 1.89-2.08 (m, 4H), 3.18-3.30 (m, 3H), 3.47-3.59 (m, 2H), 3.64-3.78 (m, 4H).

### Step 3: Preparation of toluene-4-sulfonic acid 4-methoxy-tetrahydro-pyran-4-ylmethyl ester

To a solution of (4-methoxytetrahydro-2H-pyran-4-yl) MeOH (300 mg, 2.05 mmol) in pyridine (4 mL) was added toluenesulfonic chloride (430 mg, 2.25 mmol) at room temperature and the mixture was stirred at 25° C. overnight. The mixture was concentrated under reduced pressure and the residue was dissolved in dichloromethane (2 mL). Purification by column chromatography [silica gel, 12 g, EtOAc/hexane=0/100 to 30/70] provided toluene-4-sulfonic acid 4-methoxy-tetrahydro-pyran-4-ylmethyl ester (360 mg) as a light yellow solid. ¹H NMR (300 MHz, chloroform-d) 6 [ppm]: 1.45-1.63 (m, 2H) 1.61-1.79 (m, 2H) 2.46 (s, 3H), 3.16 (s, 3H) 3.53-3.75 (m, 4H) 3.93 (s, 2H), 7.36 (d, J=8.20 Hz, 2H) 7.81 (d, J=8.20 Hz, 2H).

### Synthesis of (4-methoxytetrahydro-2H-pyran-4-yl)methanamine

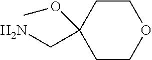

### Step 1: Preparation of 4,4-dimethoxytetrahydro-2H-pyran

A mixture of dihydro-2H-pyran-4(3H)-one (501 mg, 5 mmol), trimethyl orthoformate (0.608 mL, 5.50 mmol) and toluenesulfonic acid monohydrate (2.85 mg, 0.015 mmol) in MeOH (1 mL) was stirred in a sealed tube at 80° C. for 30 min. The reaction mixture was allowed to cool to room temperature and was concentrated under reduced pressure providing crude 4,4-dimethoxytetrahydro-2H-pyran (703 mg), which was used in the next step without further purification. ¹H NMR (400 MHz, chloroform-d) δ [ppm]: 1.61-1.90 (m, 4H) 3.20 (s, 6H) 3.60-3.78 (m, 4H).

### Step 2: Preparation of 4-methoxytetrahydro-2H-pyran-4-carbonitrile

To a solution of 4,4-dimethoxytetrahydro-2H-pyran (0.703 g, 4.81 mmol) and tin(IV)chloride (0.564 mL, 4.81 mmol) in dichloromethane (15 mL) was added slowly 2-isocyano-2-methylpropane (0.400 g, 4.81 mmol) at −70° C. and the mixture was allowed to warm to room temperature over 2-3 hrs. The mixture was diluted with aqueous sodium bicarbonate solution (10 mL) and dichloromethane (20 mL). The separated organic layer was washed with water (3×10 mL) and dried over sodium sulfate, filtered off and concentrated under reduced pressure providing crude 4-methoxytetrahydro-2H-pyran-4-carbonitrile (511 mg), which was used in the next step without further purification. GCMS: 109 [M-MeOH]; Rt=5.44 min.

### Step 3: Preparation of (4-methoxytetrahydro-2H-pyran-4-yl)methanamine

To a mixture of lithium aluminumhydride (275 mg, 7.24 mmol) in tetrahydrofuran (10 mL) at room temperature was slowly added a solution of 4-methoxytetrahydro-2H-pyran-4-carbonitrile (511 mg, 3.62 mmol) in tetrahydrofuran (10 mL). The mixture was stirred at room temperature for 1 hr and heated to reflux for 3 hrs. The reaction mixture was cooled to 0° C. and water (3 mL) was carefully added dropwise. The resulting mixture was stirred for additional 30 min and filtered to remove all solids. The filtrate was dried over sodium sulfate for 2 hrs, filtered off and concentrated under reduced pressure providing crude (4-methoxytetrahydro-2H-pyran-4-yl)methanamine (370 mg), which was used in the next step without further purification. LCMS (m/z): 146.1 [M+H]+, 114.0 [M-MeOH]; Rt=0.19 min.

### Synthesis of toluene-4-sulfonic acid 1′,1′-dioxo-hexahydro-1-thiopyran-4-yl-methyl ester

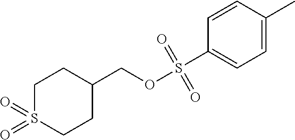

A mixture of (1′,1′-dioxo-hexahydro-1-thiopyran-4-yl)-methanol (2.5 g, 15.22 mmol) [Organic Process Research & Development 2008, 12, 892-895.], pyridine (25 mL) and tosyl-Cl (2.90 g, 15.22 mmol) was stirred for 18 hrs at 50° C. The reaction mixture was concentrated under reduced pressure. The residue was purified by column chromatography [silica gel, EtOAc/hexane=0/100 to 70/30]. Fractions were combined and concentrated under reduced pressure providing toluene-4-sulfonic acid 1′,1′-dioxo-hexahydro-1-thiopyran-4-yl-methyl ester (3.78 g). LCMS (m/z): 319.0 [M+H]+; Rt=0.71 min.

### Synthesis of 1-(tert-butoxycarbonyl)-3-fluoropiperidine-3-carboxylic acid

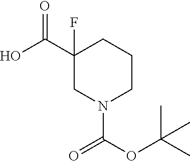

### Step 1: Preparation of 1-tert-butyl 3-methyl (3-fluoropiperidine)-1,3-dicarboxylate

To a solution of LDA [freshly prepared from BuLi (1.6M solution in hexanes, 5.14 mL, 8.22 mmol) and diisopropylamine (1.44 mL, 10.39 mmol) in tetrahydrofuran (6 mL) at 0° C.] was added dropwise a solution of 1-tert-butyl 3-methyl piperidine-1,3-dicarboxylate (2 g, 8.22 mmol) in tetrahydrofuran (8 mL) at 0° C. The solution was stirred at 0° C. for 30 min and then transferred to a 0° C. solution of N-fluorobenzenesulfonimide (3.24 g, 10.28 mmol) in tetrahydrofuran (12 mL). The reaction mixture was stirred at 0° C. for 15 min and then at room temperature for ˜20 hrs. The total solvent volume was reduced under reduced pressure to approximately one third and EtOAc was added. The mixture was washed with water, 0.1N aqueous hydrochloride solution, saturated aqueous sodium bicarbonate solution and brine. The organic phase was dried over sodium sulfate, filtered off and concentrated under reduced pressure. The crude was suspended in EtOAc and decanted. The filtrate was concentrated under reduced pressure and purified by column chromatography [silica gel, 80 g, EtOAc/heptane=0/100 to 50/50] providing 1-tert-butyl 3-methyl (3-fluoropiperidine)-1,3-dicarboxylate (775 mg) as a colorless liquid. LCMS (m/z): 262.1 [M+H]+, 206.1 [M+H, loss of t-Bu]+; Rt=0.86 min.

### Step 2: Preparation of 1-(tert-butoxycarbonyl)-3-fluoropiperidine-3-carboxylic acid

To a solution of 1-tert-butyl 3-methyl 3-fluoropiperidine-1,3-dicarboxylate (250 mg, 0.957 mmol) in MeOH (6 mL) was added slowly 2N aqueous sodium hydroxide solution (6 mL, 12.00 mmol) and the mixture was stirred for 2 hrs at room temperature. The reaction mixture was acidified with 1N aqueous hydrochloride solution and extracted with diethylether (3×). The combined organic layers were dried over sodium sulfate, filtered off and concentrated under reduced pressure providing crude 1-(tert-butoxycarbonyl)-3-fluoropiperidine-3-carboxylic acid (215 mg) as a white solid, The crude material was directly used in the next reaction without further purification. LCMS (m/z): 192.0 [M+H, loss of t-Bu]+; Rt=0.69 min.

### Synthesis of (3R,4S)-1-(benzyloxycarbonyl)-4-fluoropyrrolidine-3-carboxylic acid

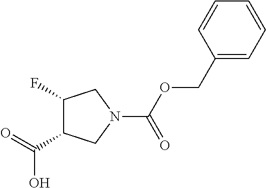

### Step 1: Preparation of (3S,4S)-benzyl 3-fluoro-4-vinylpyrrolidine-1-carboxylate

To a solution of (3R,4S)-benzyl 3-hydroxy-4-vinylpyrrolidine-1-carboxylate (5.0 g, 20.22 mmol) in (trifluoromethyl)benzene (84 mL) under argon was added diisopropylethylamine (53.0 mL, 303 mmol) and triethylamine trihydrofluoride (19.75 mL, 121 mmol). Perfluorobutanesulfonyl fluoride (PBSF) (9.09 mL, 50.5 mmol) was added slowly in five portions, each portion every in 30 min. The reaction mixture was stirred overnight. The organic solution was washed with 1N aqueous hydrochloride solution (2×), saturated aqueous sodium bicarbonate solution (2×) and water, dried over sodium sulfate, filtered off and concentrated under reduced pressure. The residue was purified by column chromatography [silica gel, 120 g, EtOAc/heptane=0/100 to 50/50] providing (3S,4S)-benzyl 3-fluoro-4-vinylpyrrolidine-1-carboxylate (3.8 g). LCMS (m/z): 250.0 [M+H]+; Rt=0.92 min.

### Step 2: Preparation of (3R,4S)-1-(benzyloxycarbonyl)-4-fluoropyrrolidine-3-carboxylic acid

A mixture of (3S,4S)-benzyl 3-fluoro-4-vinylpyrrolidine-1-carboxylate (3.8 g, 15.24 mmol), ruthenium trichloride (199 mg, 0.762 mmol), sodium periodate (13.04 g, 61.0 mmol) in carbontetrachloride (43.6 mL), water (65.3 mL) and acetonitrile (43.6 mL) was stirred overnight at room temperature. The reaction mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (3.05 g, 30.5 mmol) and 1N aqueous sulfuric acid solution (50 mL) were added. The resulting mixture was stirred at room temperature for 3 hrs. The reaction mixture was extracted with dichloromethane (2×100 mL). The combined organic layers were concentrated under reduced pressure and the residue was purified by column chromatography [silica gel] providing (3R,4S)-1-(benzyloxycarbonyl)-4-fluoropyrrolidine-3-carboxylic acid (2.9 g). LCMS (m/z): 268.0 [M+H]+; Rt=0.68 min.

### Synthesis of (3S,4S)-1-(benzyloxycarbonyl)-4-(tert-butyldiphenylsilyloxy)pyrrolidine-3-carboxylic acid

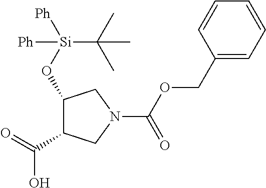

### Step 1: Preparation of (3S,4S)-benzyl 3-(4-methoxybenzoyloxy)-4-vinylpyrrolidine-1-carboxylate

A mixture of (3R,4S)-benzyl 3-hydroxy-4-vinylpyrrolidine-1-carboxylate (2.25 g, 9.10 mmol), p-anisic acid (1.66 g, 10.92 mmol), N1,N1,N2,N2-tetramethyldiazene-1,2-dicarboxamide (2.350 g, 13.65 mmol), benzene (18.20 mL) and tributyl phosphine (3.37 mL, 13.65 mmol) was stirred in a closed vial at 60° C. for 2 hrs. The reaction mixture was cooled to ambient temperature, and diluted with EtOAc (100 mL). The mixture was washed with water, brine, dried over sodium sulfate, filtered off and concentrated under reduced pressure providing (3S,4S)-benzyl 3-(4-methoxybenzoyloxy)-4-vinylpyrrolidine-1-carboxylate (2.58 g), which was directly used in the next step without further purification. LCMS (m/z): 382.2 [M+H]+; Rt=1.08 min.

### Step 2: Preparation of (3S,4S)-benzyl 3-hydroxy-4-vinylpyrrolidine-1-carboxylate

To a solution of crude (3S,4S)-benzyl 3-(4-methoxybenzoyloxy)-4-vinylpyrrolidine-1-carboxylate (2.58 g) in tetrahydrofuran (30 mL) was added 1N aqueous sodium hydroxide solution (30 mL) and the mixture was stirred at 60° C. for 18 hrs. The reaction mixture was cooled to room temperature and diluted with EtOAc (100 mL). The mixture was washed with water, brine, dried over sodium sulfate, filtered off and concentrated under reduced pressure. The residue was purified by column chromatography [silica gel] providing (3S,4S)-benzyl 3-hydroxy-4-vinylpyrrolidine-1-carboxylate (1.8 g). LCMS (m/z): 248.1 [M+H]+; Rt=0.87 min.

### Step 3: Preparation of (3S,4S)-benzyl 3-(tert-butyldiphenylsilyloxy)-4-vinylpyrrolidine-1-carboxylate

To a solution of (3S,4S)-benzyl 3-hydroxy-4-vinylpyrrolidine-1-carboxylate (1.8 g, 7.28 mmol) in dichloromethane (14 mL) was added imidazole (0.842 g, 12.37 mmol) and tert-butylchlorodiphenylsilane (2.057 mL, 8.01 mmol). The reaction mixture was stirred at room temperature for 18 hrs and filtered through a thin layer of celite. The filtrate was washed with water and brine, dried over sodium sulfate, filtered off and concentrated under reduced pressure providing crude (3S,4S)-benzyl 3-(tert-butyldiphenylsilyloxy)-4-vinylpyrrolidine-1-carboxylate (2.4 g), which was directly used in the next step without further purification. LCMS (m/z): 486.2 [M+H]+; Rt=1.44 min.

### Step 4: Preparation of (3S,4S)-1-(benzyloxycarbonyl)-4-(tert-butyldiphenylsilyloxy)-pyrrolidine-3-carboxylic acid

A mixture of (3S,4S)-benzyl 3-(tert-butyldiphenylsilyloxy)-4-vinylpyrrolidine-1-carboxylate (3.9 g, 8.03 mmol), ruthenium trichloride (0.105 g, 0.401 mmol), sodium periodate (6.87 g, 32.1 mmol) in carbontetrachloride (11.5 mL), water (17.2 mL) and acetonitrile (11.5 mL) was stirred at overnight room temperature. The mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (1.606 g, 16.06 mmol), and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure. The residue was purified by column chromatography [silica gel] providing (3S,4S)-1-(benzyloxycarbonyl)-4-(tert-butyldiphenylsilyloxy)pyrrolidine-3-carboxylic acid (2.5 g). LCMS (m/z): 504.1 [M+H]+; Rt=1.18 min.

### Synthesis of (3S,4R)-1-(benzyloxycarbonyl)-4-(tert-butyldiphenylsilyloxy)pyrrolidine-3-carboxylic acid

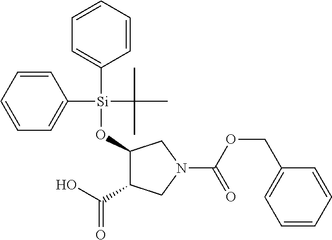

### Step 1: Preparation of benzyl 2,5-dihydro-1H-pyrrole-1-carboxylate

To a solution of 2,5-dihydro-1H-pyrrole (30 g, 434 mmol) in dioxane (1000 mL) was added CbzOSu (130 g, 521 mmol) and the mixture was stirred at room temperature for 18 hrs. The reaction mixture was concentrated to a volume of ˜300 mL and diluted with EtOAc (1000 mL). The organic layer was washed with water and brine, dried over sodium sulfate, filtered off and concentrated under reduced pressure. The residue was purified by column chromatography [silica gel] providing benzyl 2,5-dihydro-1H-pyrrole-1-carboxylate (80.0 g) as a colorless oil. Rf=0.6 (EtOAc/hexanes=30:70). ¹H NMR (400 MHz, chloroform-d) δ [ppm]: 7.32 (m, 5H), 5.80 (m, 2H), 5.77 (s, 2H), 4.22 (m, 4H). LCMS (m/z): 204.2 [M+H]+; Rt=0.86 min.

### Step 2: Preparation of benzyl 6-oxa-3-azabicyclo[3.1.0]hexane-3-carboxylate

To a solution of benzyl 2,5-dihydro-1H-pyrrole-1-carboxylate (33 g, 163 mmol) in dichloromethane (540 mL) was added MCPBA (77 wt. %, 44 g) and the reaction mixture was stirred at room temperature for 18 hrs. The mixture was diluted with saturated aqueous sodium carbonate solution (500 mL) and the resulting mixture was stirred at room temperature for 1 hr. The separated organic layer washed with water and brine, dried over sodium sulfate, filtered off and concentrated under reduced pressure. The residue was purified by column chromatography [silica gel] providing benzyl 6-oxa-3-azabicyclo[3.1.0]hexane-3-carboxylate (29.5 g) as a yellow oil. ¹H NMR (400 MHz, chloroform-d) δ [ppm]: 3.38 (dd, J=12.8, 6.0 Hz, 2H), 3.68 (d, J=3.6 Hz, 2H), 3.87 (dd, J=13.2, 19.6, 2H), 5.11 (s, 2H), 7.33 (m, 5H). LCMS (m/z): 220.0 [M+H]+; Rt=0.69 min.

### Step 3: Preparation of trans-(±)-benzyl 3-hydroxy-4-vinylpyrrolidine-1-carboxylate

To a solution of benzyl 6-oxa-3-azabicyclo[3.1.0]hexane-3-carboxylate (28.5 g, 130 mmol) and CuBrSMe₂ (26.7 g, 130 mmol) in anhydrous THF (260 mL) at −40° C. was slowly added vinyl magnesium bromide (1.0 M solution in THF, 520 mL). The reaction mixture was warmed up to −20° C. for 2 hrs. The mixture was quenched with saturated aqueous ammonium chloride solution (200 mL) and extracted with EtOAc (500 mL). The organic layer was washed with water and brine, dried over sodium sulfate, filtered off and concentrated under educed pressure. The residue was purified by column chromatography [silica gel] providing a racemic mixture of trans-(±)-benzyl 3-hydroxy-4-vinylpyrrolidine-1-carboxylate (15.5 g) as a yellow oil. Rf=0.2 (EtOAc/hexanes=30:70). ¹H NMR (400 MHz, chloroform-d) δ [ppm]: 2.71 (m, 1H) 3.28 (m, 2H) 3.72 (m, 2H) 4.11 (m, 1H) 5.14 (s, 2H) 5.16-5.23 (m, 2H) 5.69 (m, 1 H) 7.33 (m, 5H). LCMS (m/z): 248.0 [M+H]+; Rt=0.78 min.

### Step 4: Resolution of (3S,4R)-benzyl 3-hydroxy-4-vinylpyrrolidine-1-carboxylate and (3R,4S)-benzyl 3-hydroxy-4-vinylpyrrolidine-1-carboxylate

Amount: 10 g dissolved in {n-hexane:ethanol:methanol}={8:2:1}; 200 mg/mL.

### Analytical Separation:

### Column: CHIRALPAK AD (20 um) 250×4.6 mm.

Solvent: n-heptane:ethanol:methanol=8:1:1.
Flow rate: 1.0 mL/min; detection: UV=220 nm.
Fraction 1: Retention time: 9.16 min.
Fraction 2: Retention time: 13.10 min.

### Preparative Separation:

Column: CHIRALPAK AD-prep (20 um) 5 cm×50 cm.
Solvent: n-heptane:ethanol:methanol=8:1:1.
Flow rate: 100 mL/min; injection per run: 1000 mg/5 mL; detection: UV=220 nm.
Fraction 1: (3S,4R)-benzyl 3-hydroxy-4-vinylpyrrolidine-1-carboxylate. Brownish liquid. Yield: 4530 mg; ee=99.5% (UV, 220 nm).
Fraction 2: (3R,4S)-benzyl 3-hydroxy-4-vinylpyrrolidine-1-carboxylate. Brownish liquid. Yield: 4117 mg; ee=99.5% (UV, 220 nm).

### Step 5: Preparation of (3R,4S)-benzyl 3-(tert-butyldiphenylsilyloxy)-4-vinylpyrrolidine-1-carboxylate

To a solution of (3R,4S)-benzyl 3-hydroxy-4-vinylpyrrolidine-1-carboxylate (3.0 g, 12.13 mmol) in dichloromethane (24 mL) was added imidazole (1.404 g, 20.62 mmol) and tert-butylchlorodiphenylsilane (3.43 mL, 13.34 mmol). The reaction mixture was stirred at room temperature for 18 hrs and filtered through a thin layer of celite. The filtrate was washed with water and brine, dried over sodium sulfate, filtered off and concentrated under reduced pressure providing crude (3R,4S)-benzyl 3-(tert-butyldiphenylsilyloxy)-4-vinylpyrrolidine-1-carboxylate (6.2 g), which was directly used in the next step without further purification. LCMS (m/z): 486.2 [M+H]+; Rt=1.46 min.

### Step 6: Preparation of (3S,4R)-1-(benzyloxycarbonyl)-4-(tert-butyldiphenylsilyloxy)pyrrolidine-3-carboxylic acid

A mixture of (3R,4S)-benzyl 3-(tert-butyldiphenylsilyloxy)-4-vinylpyrrolidine-1-carboxylate, ruthenium trichloride (0.167 g, 0.638 mmol), sodium periodate (10.92 g, 51.1 mmol) in carbontetrachloride (18.2 mL), water (27.4 mL) and acetonitrile (18.2 mL) was stirred overnight at room temperature. The mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (2.55 g, 25.5 mmol), and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure. The residue was purified by column chromatography [silica gel] providing (3S,4R)-1-(benzyloxycarbonyl)-4-(tert-butyldiphenylsilyloxy)pyrrolidine-3-carboxylic acid (3.5 g). LCMS (m/z): 504.1 [M+H]+; Rt=1.26 min.

### Synthesis of (3R,5S)-1-(tert-butoxycarbonyl)-5-(methoxymethyl)pyrrolidine-3-carboxylic acid

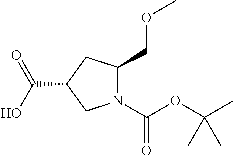

### Step 1: Preparation of (2S,4S)-4-methanesulfonyloxy-pyrrolidine-1,2-dicarboxylic acid 1-tert-butyl ester 2-methyl ester

A mixture of (2S,4S)-4-hydroxy-pyrrolidine-1,2-dicarboxylic acid 1-tert-butyl ester 2-methyl ester (5.0 g, 20.39 mmol), N,N-diisopropyl-N-ethylamine (3.16, 24.46 mmol) and methanesulfonyl chloride (2.8 g, 24.46 mmol) in dichloromethane (50 mL) was stirred at 23° C. for 18 hrs. The reaction mixture was concentrated under reduced pressure and the residue was purified by column chromatography [silica gel, 80 g, EtOAc/heptane=0/100 to 40/60] providing (2S,4S)-4-methanesulfonyloxy-pyrrolidine-1,2-dicarboxylic acid 1-tert-butyl ester 2-methyl ester (6.0 g). LCMS (m/z): 324.1 [M+H]+; Rt=0.69 min.

### Step 2: Preparation of (2S,4S)-tert-butyl 2-(hydroxymethyl)-4-(methylsulfonyloxy)pyrrolidine-1-carboxylate

To a solution of (2S,4S)-4-methanesulfonyloxy-pyrrolidine-1,2-dicarboxylic acid 1-tert-butyl ester 2-methyl ester (5.0 g) in tetrahydrofuran (31 mL) was added sodium borohydride (1.170 g, 30.9 mmol) and the mixture was heated to reflux for 3 hrs. The reaction mixture was allowed to cool to room temperature and was diluted with saturated aqueous ammonium chloride solution (5 mL) and EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2S,4S)-tert-butyl 2-(hydroxymethyl)-4-(methylsulfonyloxy)pyrrolidine-1-carboxylate (4.0 g). LCMS (m/z): 296.0 [M+H]+; Rt=0.59 min.

### Step 3: Preparation of (2S,4S)-tert-butyl 2-((tert-butyldiphenylsilyloxy)methyl)-4-(methylsulfonyloxy)pyrrolidine-1-carboxylate

To a solution of (2S,4S)-tert-butyl 2-(hydroxymethyl)-4-(methylsulfonyloxy)pyrrolidine-1-carboxylate (4.0 g, 16.18 mmol) in dichloromethane (32.4 mL) was added imidazole (1.872 g, 27.5 mmol) and tert-butylchlorodiphenylsilane (4.57 mL, 17.79 mmol). The reaction mixture was stirred at room temperature for 18 hrs and filtered through a thin layer of celite. The filtrate was washed with water and brine, dried over sodium sulfate, filtered off and concentrated under reduced pressure. The residue was purified by column chromatography [silica gel, EtOAc/heptane=0/100 to 40/60] providing (2S,4S)-tert-butyl 2-((tert-butyldiphenylsilyloxy)methyl)-4-(methylsulfonyloxy)pyrrolidine-1-carboxylate (6.0 g). LCMS (m/z): 534.5 [M+H]+; Rt=1.33 min.

### Step 4: Preparation of (2S,4R)-tert-butyl 2-((tert-butyldiphenylsilyloxy)methyl)-4-cyanopyrrolidine-1-carboxylate

To a solution of (2S,4S)-tert-butyl 2-((tert-butyldiphenylsilyloxy)methyl)-4-methylsulfonyloxy)pyrrolidine-1-carboxylate (6 g, 11.24 mmol) in DMF (50 mL) was added tetrabutylammonium cyanide (3.62 g, 13.49 mmol) and the mixture was stirred at 60° C. for 18 hrs. The reaction mixture was diluted with EtOAc (50 mL) and washed with water and brine. The organic layer was dried over sodium sulfate for ˜18 hrs, filtered off and concentrated under reduced pressure. The residue was purified by column chromatography [silica gel, EtOAc/heptane=0/100 to 50/50] providing (2S,4R)-tert-butyl 2-((tert-butyldiphenylsilyloxy)methyl)-4-cyanopyrrolidine-1-carboxylate (3.8 g). LCMS (m/z): 465.2 [M+H]+; Rt=1.37 min.

### Step 5: Preparation of (2S,4R)-tert-butyl 4-cyano-(2-hydroxymethyl)pyrrolidine-1-carboxylate

To a solution of (2S,4R)-tert-butyl 2-((tert-butyldiphenylsilyloxy)methyl)-4-cyanopyrrolidine-1-carboxylate (3.8 g, 8.18 mmol) in tetrahydrofuran (30 mL) was added tetrabutylammonium fluoride (2.57 g, 9.81 mmol) and the mixture was stirred at 23° C. for 3 hrs. The reaction mixture was concentrated under reduced pressure and the residue was dissolved in EtOAc (50 mL). The organic solution was washed with water, brine, dried over sodium sulfate, filtered off and concentrated under reduced pressure. The residue was purified by column chromatography [silica gel] providing (2S,4R)-tert-butyl 4-cyano-(2-hydroxymethyl)pyrrolidine-1-carboxylate (1.7 g).

### Step 6: Preparation of (2S,4R)-tert-butyl 4-cyano-2-(methoxymethyl)pyrrolidine-1-carboxylate

To a solution of (2S,4R)-tert-butyl 4-cyano-2-(hydroxymethyl)pyrrolidine-1-carboxylate (850 mg, 3.76 mmol) in tetrahydrofuran (20 mL) was carefully added sodium hydride (60 wt. % in mineral oil, 184 mg, 4.51 mmol) and the mixture was stirred at room temperature for 30 min. To the mixture was added methyl iodide (0.470 mL, 7.51 mmol) and stirring was continued at room temperature for 3 hrs. The reaction mixture was diluted carefully with aqueous saturated ammonium chloride solution (50 mL) and EtOAc (100 mL). The organic layer was concentrated under reduced pressure and the residue was dissolved in EtOAc (100 mL). The mixture was washed with water (2×50 mL) and brine (2×100 mL), dried over sodium sulfate, filtered off and concentrated under reduced pressure. The residue was purified by column chromatography [silica gel, EtOAc/heptane=0/100 to 60/40] providing (2S,4R)-tert-butyl 4-cyano-2-(methoxymethyl)pyrrolidine-1-carboxylate (560 mg). LCMS (m/z): 241.2 [M+H]+; Rt=0.76 min.

### Step 7: Preparation of (3R,5S)-1-(tert-butoxycarbonyl)-5-(methoxymethyl)pyrrolidine-3-carboxylic acid

A mixture of (2S,4R)-tert-butyl 4-cyano-2-(methoxymethyl)pyrrolidine-1-carboxylate (600 mg, 2.497 mmol), 6N aqueous sodium hydroxide solution (13.73 mL, 82 mmol) and EtOH (15 mL) in a closed vial was stirred at 80° C. for 1 hr. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure. The residue was purified by column chromatography [silica gel] providing (3R,5S)-1-(tert-butoxycarbonyl)-5-(methoxymethyl)pyrrolidine-3-carboxylic acid (510 mg). LCMS (m/z): 260.2 [M+H]+; Rt=0.69 min. ¹H NMR (400 MHz, methanol-d) δ [ppm]: 1.46 (s, 9H) 2.10-2.20 (m, 2H) 3.15-3.26 (m, 1H) 3.34 (s, 3H) 3.44 (d, J=4.30 Hz, 2H) 3.47-3.60 (m, 2H) 3.94-4.05 (m, 1H).

### Synthesis of 4-(tert-butoxycarbonyl)-2-methylmorpholine-2-carboxylic acid

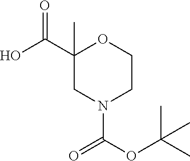

### Step 1: Preparation of 4-tert-butyl 2-methyl morpholine-2,4-dicarboxylate

To a solution of 4-(tert-butoxycarbonyl)morpholine-2-carboxylic acid (500 mg, 2.162 mmol) in MeOH (15 mL) was added sulfuric acid (10 μL, 0.188 mmol) and the reaction mixture was stirred at 70° C. for 18 hrs. The reaction mixture was allowed to cool to room temperature and diluted with 1N aqueous sodium hydroxide solution (5 mL). The mixture was concentrated under reduced pressure and the residue was dissolved in EtOAc. The solution was washed with water, brine, dried over sodium sulfate, filtered off and concentrated under reduced pressure. The residue was purified by column chromatography [silica gel] providing 4-tert-butyl 2-methyl morpholine-2,4-dicarboxylate (300 mg). LCMS (m/z): 246.1 [M+H]+; Rt=0.72 min.

### Step 2: Preparation of 2-methyl-morpholine-2,4-dicarboxylic acid 4-tert-butyl ester 2-methylester

To a solution of diisopropylamine (0.174 mL, 1.223 mmol) in tetrahydrofuran (5 mL) was added n-BuLi (0.764 mL, 1.223 mmol) at 0° C. and the mixture was stirred 0° C. for 1 hr. The mixture was cooled to −78° C. and a solution of 4-tert-butyl 2-methyl morpholine-2,4-dicarboxylate (300 mg, 1.223 mmol) in tetrahydrofuran (5 mL) was added. The reaction mixture was stirred at −78° C. for 1 hr and allowed to warm up slowly to room temperature. The mixture was diluted with saturated aqueous ammonium chloride solution (5 mL) and extracted with EtOAc (3×50 mL). The combined organic layers were washed with water and brine, dried over sodium sulfate, filtered off and concentrated under reduced pressure. The residue was purified by column chromatography [silica gel, EtOAc/heptane=0/100 to 40/60] providing 2-methyl-morpholine-2,4-dicarboxylic acid 4-tert-butyl ester 2-methylester (211 mg). LCMS (m/z): 260.0 [M+H]+; Rt=0.77 min.

### Step 3: Preparation of 4-(tert-butoxycarbonyl)-2-methylmorpholine-2-carboxylic acid

A mixture of 2-methyl-morpholine-2,4-dicarboxylic acid 4-tert-butyl ester 2-methylester (290 mg, 1.118 mmol) and 1N aqueous sodium hydroxide solution (12 mL, 12.00 mmol) in tetrahydrofuran (10 mL) was stirred at 70° C. for 2 hrs. The reaction mixture was cooled to room temperature and concentrated under reduced pressure to remove tetrahydrofuran. The aqueous solution was acidified with 1N aqueous hydrochloride solution until pH˜5 and extracted with EtOAc (3×15 mL). The organic layers were combined and washed with brine before dried over sodium sulfate, filtered off and concentrated under reduced pressure. The residue was purified by column chromatography [silica gel, EtOAc/heptane=0/100 to 70/30] providing 4-(tert-butoxycarbonyl)-2-methylmorpholine-2-carboxylic acid (155 mg). LCMS (m/z): 268.0 [M+Na]+; Rt=0.61 min.

### Synthesis of (3R,5S)-/(3S,5R)-1-(benzyloxycarbonyl)-5-methylpiperidine-3-carboxylic acid [mixture of cis isomers] and (3R,5R)-/(3S,5S)-1-(benzyloxycarbonyl)-5-methylpiperidine-3-carboxylic acid [mixture of trans isomers]

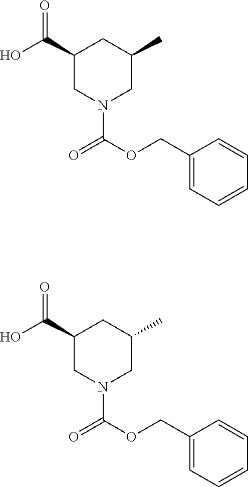

### Step 1: Preparation of methyl 5-methylpiperidine-3-carboxylate (mixture of cis and trans isomers)

A mixture of methyl 5-methylnicotinate (1.06 g, 7.01 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 16 hrs. The reaction mixture was filtered through a pad of celite and washed with MeOH (150 mL). The filtrate was concentrated under reduced pressure providing crude methyl 5-methylpiperidine-3-carboxylate (1.5 g; mixture of cis and trans isomers) as a colorless oil, which was directly used in the next step without further purification. LCMS (m/z): 158.1 [M+H]+; Rt=0.32 min.

### Step 2: Preparation of (3R,5S)-/(3S,5R)-5-methyl-piperidine-1,3-dicarboxylic acid 1-benzyl ester 3-methyl ester [cis isomers] and (3R,5R)-/(3S,5S)-5-methyl-piperidine-1,3-dicarboxylic acid 1-benzyl ester 3-methyl ester [trans isomers]

To a mixture of crude methyl 5-methylpiperidine-3-carboxylate (1.5 g, 7.01 mmol) and aqueous sodium carbonate solution (10 wt. %; 20 mL) in tetrahydrofuran (40 mL) was slowly added benzylchloroformate (1.491 mL, 10.45 mmol). The reaction mixture was stirred at 25° C. for 16 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 60/40]providing a mixture of the cis isomers (3R,5S)-/(3S,5R)-5-methyl-piperidine-1,3-dicarboxylic acid 1-benzyl ester 3-methyl ester (1.66 g) as colorless oil and a mixture of the trans isomers (3R,5R)-/(3S,5S)-5-methyl-piperidine-1,3-dicarboxylic acid 1-benzyl ester 3-methyl ester (1.52 g) as colorless oil.

Cis isomers: LCMS (m/z): 292.1 [M+H]+; Rt=0.99 min. Analytical HPLC: Rt=4.04 min.

¹H NMR (300 MHz, chloroform-d) δ [ppm]: 0.92 (d, J=6.45 Hz, 3H) 1.21 (q, J=12.41 Hz, 1H) 1.60 (br. s., 1H) 2.11 (d, J=13.19 Hz, 1H) 2.29 (br. s., 1H) 2.43-2.57 (m, 1 H) 2.75 (br. s., 1H) 3.69 (s, 3H) 4.14 (br. s., 1H) 4.42 (br. s., 1H) 5.14 (br. s., 2H) 7.36 (s, 5H).

Trans isomers: LCMS (m/z): 292.1 [M+H]+; Rt=0.96 min. Analytical HPLC: Rt=3.85 min.

¹H NMR (300 MHz, chloroform-d) δ [ppm]: 0.92 (d, J=6.74 Hz, 3H) 1.47 (br. s., 1H) 1.88-2.07 (m, 2H) 2.67 (br. s., 1H) 2.80-3.09 (m, 1H) 3.30-4.08 (m, 6H) 5.13 (q, J=12.31 Hz, 2H) 7.29-7.39 (m, 5H).

### Step 3-a: Preparation of (3R,5S)-/(3S,5R)-1-(benzyloxycarbonyl)-5-methylpiperidine-3-carboxylic acid [cis isomers]

To the mixture of (3R,5S)-/(3S,5R)-5-methyl-piperidine-1,3-dicarboxylic acid 1-benzyl ester 3-methyl ester (1.66 g, 5.70 mmol) in MeOH (4.5 mL) and water (3 mL) was added 6N aqueous sodium hydroxide solution (1.5 mL, 9.0 mmol). The reaction mixture was stirred at 25° C. for 2 hrs and concentrated under reduced pressure to a volume of −2 mL. The mixture was acidified with 1N aqueous hydrochloride solution until pH˜4, diluted with EtOAc and stirred for 10 min. The separated organic layer was washed with brine, dried sodium sulfate, filtered off and concentrated under reduced pressure providing a mixture of the cis isomers (3R,5S)- and (3S,5R)-1-(benzyloxycarbonyl)-5-methylpiperidine-3-carboxylic acid (1.36 g) as a colorless oil, which was directly used in the next step without further purification. LCMS (m/z): 278.1 [M+H]+; Rt=0.81 min.

### Step 3-b: Preparation of (3R,5R)-/(3S,5S)-1-(benzyloxycarbonyl)-5-methylpiperidine-3-carboxylic acid [trans isomers]

To the mixture of (3R,5S)-/(3S,5R)-5-methyl-piperidine-1,3-dicarboxylic acid 1-benzyl ester 3-methyl ester (1.55 g, 5.32 mmol) in MeOH (4.5 mL) and water (3 mL) was added 6N aqueous sodium hydroxide solution (1.5 mL, 9.0 mmol). The reaction mixture was stirred at 25° C. for 2 hrs and concentrated under reduced pressure to a volume of ˜2 mL. The mixture was acidified with 1N aqueous hydrochloride solution until pH˜4, diluted with EtOAc and stirred for 10 min. The separated organic layer was washed with brine solution, dried over sodium sulfate, filtered off and concentrated under reduced pressure providing a mixture of trans isomers (3R,5R)- and (3S,5S)-1-(benzyloxycarbonyl)-5-methylpiperidine-3-carboxylic acid (1.22 g) as a colorless oil, which was directly used in the next step without further purification. LCMS (m/z): 278.1 [M+H]+; Rt=0.79 min.

### Synthesis of (3S,4R)-1-(benzyloxycarbonyl)-4-methoxypyrrolidine-3-carboxylic acid

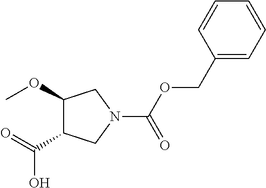

### Step 1: Preparation of (3R,4S)-benzyl-3-methoxy-4-vinylpyrrolidine-1-carboxylate

To a solution of (3R,4S)-benzyl 3-hydroxy-4-vinylpyrrolidine-1-carboxylate (5.3 g, 21.43 mmol) in DMF (25 mL) was added carefully sodium hydride (60 wt. % in mineral oil, 1.714 g, 42.9 mmol) and the mixture was stirred at room temperature for 1 hr. To the mixture was added methyl iodide (4.29 mL, 68.6 mmol) slowly over 30 min and stirring was continued for additional 18 hrs at 25° C. The mixture was diluted with saturated aqueous ammonium chloride solution (10 mL) and with EtOAc (100 mL). The mixture was washed with water and brine, dried over sodium sulfate, filtered off and concentrated under reduced pressure. The residue was purified by column chromatography [silica gel, EtOAc/heptane=0/100 to 50/50] providing (3R,4S)-benzyl-3-methoxy-4-vinylpyrrolidine-1-carboxylate (5.0 g). LCMS (m/z): 262.1 [M+H]+; Rt=0.78 min.

### Step 2: Preparation of (3S,4R)-1-(benzyloxycarbonyl)-4-methoxypyrrolidine-3-carboxylic acid

A mixture of (3R,4S)-benzyl-3-methoxy-4-vinylpyrrolidine-1-carboxylate (5 g, 19.13 mmol), ruthenium trichloride (4.99 g, 19.13 mmol), sodium periodate (16.37 g, 77 mmol) in carbontetrachloride (20 mL), water (20 mL) and acetonitrile (20 mL) was stirred at room temperature overnight. The reaction mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (3.05 g, 30.5 mmol) and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure and the residue was purified by column chromatography [silica gel] providing (3R,4S)-1-(benzyloxycarbonyl)-4-methoxypyrrolidine-3-carboxylic acid (2.7 g). LCMS (m/z): 280.0 [M+H]+; Rt=0.69 min.

### Synthesis of (3R,5R)-1-(tert-butoxycarbonyl)-5-(methoxymethyl)pyrrolidine-3-carboxylic acid

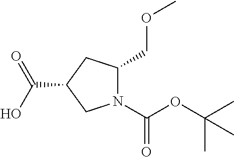

### Step 1: Preparation of (2R,4R)-4-(tert-butyl-diphenyl-silanyloxy)-pyrrolidine-1,2-dicarboxylic acid 1-tert-butyl ester 2-methyl ester

To a solution of (2R,4R)-4-hydroxy-pyrrolidine-1,2-dicarboxylic acid 1-tert-butyl ester 2-methyl ester (5.0 g, 20.22 mmol) in dichloromethane (35 mL) was added imidazole (2.34 g, 34.4 mmol) and tert-butylchlorodiphenylsilane (5.71 mL, 22.24 mmol). The reaction mixture was stirred at room temperature for 18 hrs and filtered through a thin layer of celite. The filtrate was washed with water and brine, dried over sodium sulfate, filtered off and concentrated under reduced pressure providing crude (2R,4R)-4-(tert-butyl-diphenyl-silanyloxy)-pyrrolidine-1,2-dicarboxylic acid 1-tert-butyl ester 2-methyl ester (10.9 g), which was directly used in the next step without further purification. LCMS (m/z): 486.2 [M+H]+; Rt=1.36 min.

### Step 2: Preparation of (2R,4R)-tert-butyl 4-(tert-butyldiphenylsilyloxy)-2-(hydroxymethyl)pyrrolidine-1-carboxylate

To a solution of (2R,4R)-1-tert-butyl 2-methyl 4-(tert-butyldiphenylsilyloxy)pyrrolidine-1,2-dicarboxylate (10.0 g, 20.68 mmol) in tetrahydrofuran (100 mL) was added sodium borohydride (1.564 g, 41.4 mmol) and the mixture was heated at 70° C. for 2 hrs. The reaction mixture was allowed to cool to room temperature and was diluted with saturated aqueous ammonium chloride solution (5 mL) and EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2R,4R)-tert-butyl 4-(tert-butyldiphenylsilyloxy)-2-(hydroxymethyl)pyrrolidine-1-carboxylate (5.0 g). LCMS (m/z): 456.2 [M+H]+; Rt=0.88 min.

### Step 3: Preparation of (2R,4R)-tert-butyl 4-(tert-butyldiphenylsilyloxy)-2-(methoxymethyl)pyrrolidine-1-carboxylate

To a solution of (2R,4R)-tert-butyl 4-(tert-butyldiphenylsilyloxy)-2-(hydroxymethyl)pyrrolidine-1-carboxylate (5.0 g, 10.97 mmol) in tetrahydrofuran (25 mL) was added carefully sodium hydride (0.316 g, 13.17 mmol) and the mixture was stirred at room temperature for 2 hrs. To the mixture was added methyl iodide (1.372 mL, 21.95 mmol) and stirring was continued at 23° C. for 183 hrs. The reaction mixture was diluted carefully with aqueous saturated ammonium chloride solution (10 mL) and EtOAc (100 mL). The mixture was washed with water (2×50 mL) and brine (2×100 mL), dried over sodium sulfate, filtered off and concentrated under reduced pressure. The residue was purified by column chromatography [silica gel, EtOAc/heptane=0/100 to 40/60] providing (2R,4R)-tert-butyl 4-(tert-butyldiphenylsilyloxy)-2-(methoxymethyl)pyrrolidine-1-carboxylate (4.7 g). LCMS (m/z): 470.1 [M+H]+; Rt=1.45 min.

### Step 4: Preparation of (2R,4R)-tert-butyl 4-hydroxy-2-(methoxymethyl)pyrrolidine-1-carboxylate

To a solution of (2R,4R)-tert-butyl 4-(tert-butyldiphenylsilyloxy)-2-(methoxymethyl)pyrrolidine-1-carboxylate (4.60 g, 9.79 mmol) in tetrahydrofuran (30 mL) was added tetrabutylammonium fluoride (2.56 g, 9.79 mmol) and the mixture was stirred at 23° C. for 2 hrs. The reaction mixture was diluted with EtOAc (100 mL) and washed with water, brine, dried over sodium sulfate, filtered off and concentrated under reduced pressure. The residue was purified by column chromatography [silica gel, 400 g, EtOAc/heptane=0/100 to 50/50] providing (2R,4R)-tert-butyl 4-hydroxy-2-(methoxymethyl)pyrrolidine-1-carboxylate (1.0 g). LCMS (m/z): 232.1 [M+H]+; Rt=0.62 min.

### Step 5: Preparation of (2R,4S)-tert-butyl 4-(4-methoxybenzoyloxy)-2-(methoxymethyl)pyrrolidine-1-carboxylate

A mixture of (2R,4R)-tert-butyl 4-hydroxy-2-(methoxymethyl)pyrrolidine-1-carboxylate (1 g, 4.32 mmol), p-anisic acid (0.789 g, 5.19 mmol), N1,N1,N2,N2-tetramethyldiazene-1,2-dicarboxamide (0.744 g, 4.32 mmol), benzene (20 mL) and tributyl phosphine (1.60 mL, 6.49 mmol) in a closed vial was stirred at 60° C. for 2 hrs. The reaction mixture was diluted with EtOAc (100 mL). The mixture was washed with water, brine, dried over sodium sulfate, filtered off and concentrated under reduced pressure. The residue was purified by column chromatography [silica gel] providing (2R,4S)-tert-butyl 4-(4-methoxybenzoyloxy)-2-(methoxymethyl)pyrrolidine-1-carboxylate. (1.2 g). LCMS (m/z): 366.2 [M+H]+; Rt=1.02 min.

### Step 6: Preparation of (2R,4S)-tert-butyl 4-hydroxy-2-(methoxymethyl)pyrrolidine-1-carboxylate

To a solution of (2R,4S)-tert-butyl 4-(4-methoxybenzoyloxy)-2-(methoxymethyl)pyrrolidine-1-carboxylate (1.2 g, 3.28 mmol) in tetrahydrofuran (20 mL) was added 3N aqueous sodium hydroxide solution (20 mL) and the mixture was stirred at 70° C. for 18 hrs. The reaction mixture was allowed to cool to room temperature and diluted with water (50 mL). The mixture was extracted with EtOAc (2×100 mL). The combined organic layers were washed with water (50 mL), brine (2×100 mL), dried over sodium sulfate, filtered off and concentrated under reduced pressure. The residue was purified by column chromatography [silica gel] providing (2R,4S)-tert-butyl 4-hydroxy-2-(methoxymethyl)pyrrolidine-1-carboxylate (600 mg). LCMS (m/z): 232.1 [M+H]+; Rt=0.62 min.

### Step 7: Preparation of (2R,4S)-tert-butyl 2-(methoxymethyl)-4-(methylsulfonyloxy)pyrrolidine-1-carboxylate

A mixture of (2R,4S)-tert-butyl 4-hydroxy-2-(methoxymethyl)pyrrolidine-1-carboxylate (600 mg, 2.59 mmol), N,N-diisopropyl-N-ethylamine (0.544 mL, 3.11 mmol) and methanesulfonyl chloride (357 mg, 3.11 mmol) in dichloromethane (10 mL) was stirred at 23° C. for 18 hrs. The reaction mixture was concentrated under reduced pressure and the residue was purified by column chromatography [silica gel] (2R,4S)-tert-butyl 2-(methoxymethyl)-4-(methylsulfonyloxy)pyrrolidine-1-carboxylate (650 mg). LCMS (m/z): 310.1 [M+H]+; Rt=0.90 min.

### Step 8: Preparation of (2R,4R)-tert-butyl 4-cyano-2-(methoxymethyl)pyrrolidine-1-carboxylate

To a solution of (2R,4S)-tert-butyl 2-(methoxymethyl)-4-(methylsulfonyloxy)pyrrolidine-1-carboxylate (910 mg, 2.94 mmol) in DMF (15 mL) was added tetrabutylammonium cyanide (948 mg, 3.53 mmol) and the mixture was stirred at 60° C. for 18 hrs. The reaction mixture was diluted with EtOAc (50 mL) and washed with water (2×) and brine. The organic layer was dried over sodium sulfate, filtered off and concentrated under reduced pressure. The residue was purified by column chromatography [silica gel, EtOAc/heptane=0/100 to 50/50] providing (2R,4R)-tert-butyl 4-cyano-2-(methoxymethyl)pyrrolidine-1-carboxylate (250 mg). LCMS (m/z): 185.0 [M+H, loss of t-Bu]+; Rt=0.78 min.

### Step 9: Preparation of (3R,5R)-1-(tert-butoxycarbonyl)-5-(methoxymethyl)pyrrolidine-3-carboxylic acid

A mixture of (2R,4R)-tert-butyl 4-cyano-2-(methoxymethyl)pyrrolidine-1-carboxylate (250 mg, 1.040 mmol), 6N aqueous sodium hydroxide solution (5.72 mL, 34.3 mmol) and EtOH (7 mL) in a closed vial was stirred at 85° C. for 30 min. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure providing crude (3R,5R)-1-(tert-butoxycarbonyl)-5-(methoxymethyl)pyrrolidine-3-carboxylic acid (210 mg), which was directly used in the next step without further purification. LCMS (m/z): 282.0 [M+Na]+; Rt=0.68 min. ¹H NMR (400 MHz, methanol-d4) δ [ppm]: 1.46 (s, 9 H) 2.08-2.22 (m, 2H) 3.15-3.27 (m, 1H) 3.34 (s, 3H) 3.44 (d, J=4.70 Hz, 2H) 3.46-3.61 (m, 2H) 3.94-4.05 (m, 1H).

### Synthesis of 1-(benzyloxycarbonyl)-5-fluoropiperidine-3-carboxylic acid [cis isomers]

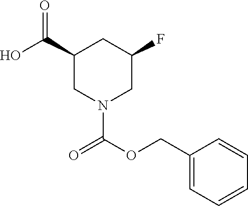

### Step 1: Preparation of 1-benzyl-5-hydroxypiperidine-3-carboxylic acid

To a mixture of 5-hydroxypiperidine-3-carboxylic acid (3 g, 20.67 mmol) and potassium carbonate (4.41 g, 31.9 mmol) in MeOH (48 mL) and water (24 mL) was added slowly a solution of benzyl bromide (2.58 mL, 21.70 mmol) in MeOH (2.00 mL). The mixture was stirred for ˜3 hrs at room temperature. The volatile solvent was removed under reduced pressure and the remaining solution was carefully acidified with 1N aqueous hydrochloride solution (˜100 mL). The aqueous solution was concentrated under reduced pressure to dryness. The residue was suspended in MeOH (˜50 mL) and filtered off. To the filtrate was added sodium methoxide in MeOH (25 wt. %, 6.8 g) and the reaction mixture was stirred for ˜18 hrs. The mixture was filtered and concentrated under reduced pressure providing crude 1-benzyl-5-hydroxypiperidine-3-carboxylic acid as a solid, which was directly used in the next reaction without further purification. LCMS (m/z): 336.0 [M+H]+; Rt=0.36 min.

### Step 2: Preparation of methyl 1-benzyl-5-hydroxypiperidine-3-carboxylate

Chlorotrimethylsilane (17.11 mL, 134 mmol) was added slowly to a solution of crude 1-benzyl-5-hydroxypiperidine-3-carboxylic acid (4.5 g, 19.13 mmol) in MeOH (90 mL). The mixture was stirred for ˜18 hrs and concentrated under reduced pressure. The residue was purified by column chromatography [silica gel, 80 g, 30 min, EtOAc/heptane=20/80 to 70/30] providing methyl 1-benzyl-5-hydroxypiperidine-3-carboxylate (3.37 g, 71% over 2 steps) as a colorless oil. LCMS (m/z): 250.3 [M+H]+; Rt=0.36 min.

### Step 3: Preparation of a mixture of (3S,5R)-/(3R,5S)-methyl 1-benzyl-5-fluoropiperidine-3-carboxylate [cis isomers] and (3R,5R)-/(3S,5S)-methyl 1-benzyl-5-(fluoromethyl)pyrrolidine-3-carboxylate [cis isomers]

To methyl 1-benzyl-5-hydroxypiperidine-3-carboxylate (2 g, 8.02 mmol) in DCM (32 mL) at −78° C. was added dropwise DAST (2.12 mL, 16.04 mmol). The mixture was allowed to warm slowly to room temperature over ˜16 hrs. The reaction mixture was diluted with saturated aqueous sodium bicarbonate solution. The separated aqueous layer was extracted with dichloromethane (2×). The combined organic layers were concentrated under reduced pressure. The residue was purified by column chromatography [silica gel, 40 g, 30 min, EtOAc/heptane=0/100 to 40/60] providing a mixture of methyl 1-benzyl-5-fluoropiperidine-3-carboxylate [cis isomers] and methyl 1-benzyl-5-(fluoromethyl)pyrrolidine-3-carboxylate [cis isomers] (1.80 g) as a slightly orange oil. LCMS (m/z): 252.1 [M+H]+; Rt=0.41 min.

### Step 4: Preparation of mixture of methyl 5-fluoropiperidine-3-carboxylate acetic acid salt [cis isomers] and methyl 5-(fluoromethyl)pyrrolidine-3-carboxylate acetic acid salt [cis isomers]

To the mixture of methyl 1-benzyl-5-fluoropiperidine-3-carboxylate [cis isomers] and methyl 1-benzyl-5-(fluoromethyl)pyrrolidine-3-carboxylate [cis isomers] (1.8 g, 7.16 mmol) in acetic acid (14 mL) was added Pd/C (10 wt. %, 170 mg) and platinum(IV)oxide (240 mg, 1.057 mmol). The mixture was hydrogenated in a steel bomb for ˜16 hrs (pressure: 1400 psi). The catalyst was filtered off through celite and the clear solution was concentrated under reduced pressure providing crude mixture of methyl 5-fluoropiperidine-3-carboxylate acetic acid salt [cis isomers] and methyl 5-(fluoromethyl)pyrrolidine-3-carboxylate acetic acid salt [cis isomers] as a slightly yellowish oil, which was directly used in the next reaction without further purification. LCMS (m/z): 162.0 [M+H]+; Rt=0.19 min.

### Step 5: Preparation of (3R,5S)-/(3S,5R)-5-fluoro-piperidine-1,3-dicarboxylic acid 1-benzyl ester 3-methyl ester [cis isomers] and (3R,5R)/(3S,5S)-5-fluoromethyl-pyrrolidine-1,3-dicarboxylic acid 1-benzyl ester 3-methyl ester [cis isomers]

To a mixture of crude methyl 5-fluoropiperidine-3-carboxylate (1.584 g, 7.16 mmol) acetic acid salt in tetrahydrofuran (15 mL) was added aqueous sodium carbonate solution (10 wt. %, ˜7 mL) until pH˜8-9. Benzyl chloroformate (1.145 mL, 8.02 mmol) was added slowly and saturated aqueous sodium bicarbonate solution was added. The reaction mixture was stirred for 1 hr and was diluted with EtOAc. The separated organic phase was washed with saturated aqueous sodium bicarbonate solution (2×) and concentrated under reduced pressure. The residue was dissolved in EtOAc, dried over sodium sulfate, filtered off and concentrated under reduced pressure. The residue was purified by column chromatography [silica gel, 40 g, 16 min, EtOAc/heptane=0/100 to 40/60]. Fractions were combined and concentrated under reduced pressure providing Fraction 1: 1.005 g (ratio of isomers: ˜90:10); Fractions 2: 459 mg (ratio of isomers: ˜50:50). Fractions 2 was dissolved in DMSO and purified by HPLC [˜50 mg/l mL of DMSO]. Fractions of P1 and P2 were collected and lyophilized providing cis isomers and trans isomers of 1-benzyl 3-methyl 5-fluoropiperidine-1,3-dicarboxylate as colorless oils.

Fraction 1/Fraction P1: 5-Fluoro-piperidine-1,3-dicarboxylic acid 1-benzyl ester 3-methyl ester [cis isomers]

Yield: 143 mg; LCMS (m/z): 296.0 [M+H]+; Rt=0.83 min. ¹H NMR (400 MHz, DMSO-d6, 70° C.) δ [ppm]: 7.21-7.48 (m, 5H), 5.07-5.15 (m, 2H), 4.54-4.76 (m, 1 H), 3.75-3.95 (m, 2H), 3.58-3.63 (m, 3H), 3.26-3.38 (m, 1H), 3.17-3.27 (m, 1H), 2.68 (ttd, J=9.2, 4.5, 1.6 Hz, 1H), 2.27 (ddt, J=17.6, 13.2, 4.2 Hz, 1H), 1.89 (br. s., 1H)

Fraction P2: 5-Fluoromethyl-pyrrolidine-1,3-dicarboxylic acid 1-benzyl ester 3-methyl ester [cis isomers]

Yield: 118 mg; LCMS (m/z): 296.0 [M+H]+; Rt=0.85 min. ¹H NMR (400 MHz, DMSO-d6, 70° C.) δ [ppm]: 7.14-7.58 (m, 5H), 5.09 (d, J=5.0 Hz, 2H), 4.46-4.64 (m, 1H), 4.40 (d, J=3.4 Hz, 1H), 3.96-4.15 (m, 1H), 3.80 (dd, J=10.6, 8.2 Hz, 1H), 3.35-3.49 (m, 1H), 3.16 (quin, J=8.0 Hz, 1H), 3.09 (s, 3H), 2.26-2.45 (m, 1H), 2.04-2.13 (m, 1H)

### Step 6: Preparation of (3R,5S)-/(3S,5R)-1-(benzyloxycarbonyl)-5-fluoropiperidine-3-carboxylic acid [cis isomers]

To a solution of Fraction 1 (5-fluoro-piperidine-1,3-dicarboxylic acid 1-benzyl ester 3-methyl ester [cis isomers]; 500 mg, 1.693 mmol) in MeOH (10 mL) was added slowly 2N aqueous sodium hydroxide solution (10 mL). The mixture was stirred for ˜10 min at room temperature. The mixture was acidified with 1N aqueous hydrochloride solution and the volatile solvent was removed under reduced pressure. The residue was diluted with EtOAc. The separated organic layer was washed with brine, dried over sodium sulfate, filtered off and concentrated under reduced pressure providing crude mixture of (3R,5S)-/(3S,5R)-1-(benzyloxycarbonyl)-5-fluoropiperidine-3-carboxylic acid [cis isomers] (487 mg) as a white solid, which was directly used in the next reaction without further purification. LCMS (m/z): 282.0 [M+H]+; Rt=0.70 min.

### Synthesis of (3S,5S)-/(3R,5R)-1-(benzyloxycarbonyl)-5-(fluoromethyl)pyrrolidine-3-carboxylic acid [cis isomers]

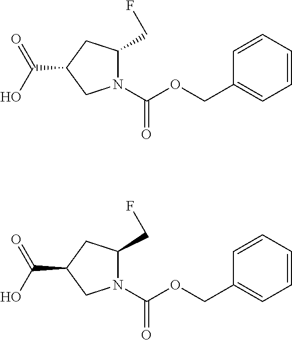

To a solution of Fraction P2 (5-fluoromethyl-pyrrolidine-1,3-dicarboxylic acid 1-benzyl ester 3-methyl ester [cis isomers]; 70 mg, 0.237 mmol) in MeOH (8 mL) was added slowly 2N aqueous sodium hydroxide solution (8 mL). The mixture was stirred for ˜5 min at room temperature. The mixture was partially concentrated under reduced pressure and was acidified with 1N aqueous hydrochloride solution and diluted with EtOAc. The separated aqueous layer was extracted with EtOAc (2×). The combined organic layers were dried over sodium sulfate, filtered off and concentrated under reduced pressure providing crude mixture of (3S,5S)-/(3R,5R)-1-(benzyloxycarbonyl)-5-(fluoromethyl)pyrrolidine-3-carboxylic acid [cis isomers] (56 mg) as a colorless oil, which was directly used in the next reaction without further purification. LCMS (m/z): 282.1 [M+H]+; Rt=0.71 min.

### Synthesis of (3R,5S)-/(3S,5R)-1-(benzyloxycarbonyl)-5-(trifluoromethyl)piperidine-3-carboxylic acid and (3R,5R)-/(3S,5S)-1-(benzyloxycarbonyl)-5-(trifluoromethyl)piperidine-3-carboxylic acid

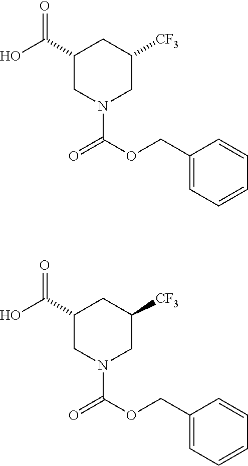

### Step 1: Preparation of methyl 5-(trifluoromethyl)nicotinate

To a solution of 5-(trifluoromethyl)nicotinic acid (1.0 g, 5.08 mmol) in MeOH (10 mL) was added slowly thionyl chloride (0.926 mL, 12.69 mmol). The reaction mixture was stirred at 45° C. for 18 hrs and then concentrated under reduced pressure. The residue was dissolved in dichloromethane and the organic layer was washed with saturated aqueous sodium bicarbonate solution, water and brine, dried over sodium sulfate, filtered off and concentrated under reduced pressure providing crude methyl 5-(trifluoromethyl)nicotinate (736 mg) as oil, which was directly used in the next step without further purification. LCMS (m/z): 206.0 [M+H]+; Rt=0.72 min.

### Step 2: Preparation of methyl 5-(trifluoromethyl)piperidine-3-carboxylate (mixture of cis and trans isomers)

A mixture of methyl 5-(trifluoromethyl)nicotinate (736 mg, 3.59 mmol), Pd/C (10 wt. %, 36 mg) and platinum(IV)oxide (52.5 mg, 0.231 mmol) in acetic acid (11 mL) was stirred in a steel bomb under hydrogen atmosphere (200 psi) at 25° C. for 20 hrs. The reaction mixture was filtered through a pad of celites and washed with MeOH (50 mL). The filtrate was concentrated under reduced pressure providing crude methyl 5-(trifluoromethyl)piperidine-3-carboxylate (936 mg; mixture of cis and trans isomers) as a colorless oil, which was directly used in the next step without further purification. LCMS (m/z): 212.0 [M+H]+; Rt=0.38 min.

### Step 3: Preparation of (3R,5S)-/(3S,5R)-5-trifluoromethyl-piperidine-1,3-dicarboxylic acid 1-benzyl ester 3-methyl ester [cis isomers] and (3R,5R)-/(3S,5S)-5-trifluoromethyl-piperidine-1,3-dicarboxylic acid 1-benzyl ester 3-methyl ester [trans isomers]

To a mixture of crude methyl 5-(trifluoromethyl)piperidine-3-carboxylate (953 mg, 3.61 mmol) aqueous sodium carbonate solution (10 wt. %; 5.13 mL) in tetrahydrofuran (15 mL) was added slowly benzylchloroformate (0.58 mL, 4.04 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine solution. The organic phase was dried over sodium sulfate, filtered off and concentrated under reduced pressure. The residue was purified by column chromatography [silica gel, 24 g, EtOAc/heptane=0/100 to 30/70] providing a mixture of the cis isomers (3R,5S)-/(3S,5R)-5-trifluoromethyl-piperidine-1,3-dicarboxylic acid 1-benzyl ester 3-methyl ester (296 mg) as a white solid and a mixture of the trans isomers (3R,5R)-/(3S,5S)-5-trifluoromethyl-piperidine-1,3-dicarboxylic acid 1-benzyl ester 3-methyl ester (240 mg) as an oil.

Cis isomers: LCMS (m/z): 346.0 [M+H]+; Rt=1.01 min. Analytical HPLC: Rt=4.22 min.

Trans isomers: LCMS (m/z): 346.1 [M+H]+; Rt=0.98 min. Analytical HPLC: Rt=4.09 min.

### Step 4-a: Preparation of (3R,5S)-/(3S,5R)-1-(benzyloxycarbonyl)-5-(trifluoromethyl)piperidine-3-carboxylic acid [cis isomers]

To a mixture of the cis isomers (3R,5S)-/(3S,5R)-1-benzyl 3-methyl 5-(trifluoromethyl)piperidine-1,3-dicarboxylate (296 mg, 0.857 mmol) in MeOH (0.9 mL) and water (0.6 mL) was added 6N aqueous sodium hydroxide solution (0.3 mL, 1.8 mmol). The reaction mixture was stirred at 25° C. for 1 hr and concentrated under reduced pressure to a volume of ˜0.5 mL. The mixture was acidified with 1N hydrochloride solution until pH˜4, diluted with EtOAc and stirred for 10 min. The separated organic layer was washed with brine solution, dried over sodium sulfate, filtered off and concentrated under reduced pressure providing a mixture of (3R,5S)- and (3S,5R)-1-(benzyloxycarbonyl)-5-(trifluoromethyl)piperidine-3-carboxylic acid (254 mg) as a colorless oil, which was directly used in the next step without further purification. LCMS (m/z): 332.0 [M+H]+; Rt=0.91 min.

### Step 4-b: Preparation of (3R,5R)-/(3S,5S)-1-(benzyloxycarbonyl)-5-(trifluoromethyl)piperidine-3-carboxylic acid [trans isomers]

To a mixture of the trans isomers (3R,5R)-/(3S,5S)-1-benzyl 3-methyl 5-(trifluoromethyl)piperidine-1,3-dicarboxylate (1.55 g, 5.32 mmol) in MeOH (0.75 mL) and water (0.5 mL) was added 6N aqueous sodium hydroxide solution (0.25 mL, 1.5 mmol). The reaction mixture was stirred at 25° C. for 2 hrs and concentrated under reduced pressure to a volume of ˜0.5 mL. The mixture was acidified with 1N hydrochloride until pH˜4, diluted with EtOAc and stirred for 10 min. The separated organic layer was washed with brine, dried over sodium sulfate, filtered off and concentrated under reduced pressure providing a mixture of (3R,5R)-/(3S,5S)-1-(benzyloxycarbonyl)-5-(trifluoromethyl)piperidine-3-carboxylic acid (218 mg) as a colorless oil, which was directly used in the next step without further purification. LCMS (m/z): 332.1 [M+H]+; Rt=0.83 min

### Synthesis of (3R,6S)-/(3S,6R)-1-(benzyloxycarbonyl)-6-methylpiperidine-3-carboxylic acid and (3R,6R)-/(3S,6S)-1-(benzyloxycarbonyl)-6-methylpiperidine-3-carboxylic acid

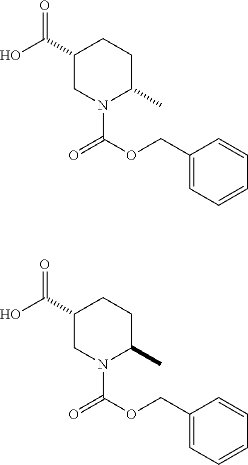

### Step 1: Preparation of methyl 6-methylpiperidine-3-carboxylate (mixture of cis and trans isomers)

A mixture of methyl 6-methylnicotinate (1.52 g, 10 mmol), Pd/C (10 wt. %, 100 mg) and platinum(IV)oxide (150 mg, 0.661 mmol) in acetic acid (16 mL) was stirred in a steel bomb under hydrogen atmosphere (200 psi) at 25° C. for 16 hrs. The reaction mixture was filtered through a pad of celites and washed with MeOH (150 mL). The filtrate was concentrated under reduced pressure providing crude methyl 6-methylpiperidine-3-carboxylate (2.5 g; mixture of cis and trans isomers) as a colorless oil, which was directly used in the next step without further purification. LCMS (m/z): 158.1 [M+H]+; Rt=0.28 min.

### Step 2: Preparation of (3R,6S)-/(3S,6R)-6-methyl-piperidine-1,3-dicarboxylic acid 1-benzyl ester 3-methyl ester [cis isomers] and (3R,6R)-/(3S,6S)-6-methyl-piperidine-1,3-dicarboxylic acid 1-benzyl ester 3-methyl ester [trans isomers]

To a mixture of crude methyl 6-methylpiperidine-3-carboxylate (2.33 g, 10 mmol) aqueous sodium carbonate solution (10 wt. %; 20 mL) in tetrahydrofuran (40 mL) was added slowly benzylchloroformate (1.431 mL, 10.03 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 40/60] providing a mixture of the cis isomers (3R,6S)-/(3S,6R)-6-methyl-piperidine-1,3-dicarboxylic acid 1-benzyl ester 3-methyl ester (1.74 g) as colorless oil and a mixture of the trans isomers (3R,6R)-/(3S,6S)-6-methyl-piperidine-1,3-dicarboxylic acid 1-benzyl ester 3-methyl ester (0.725 g) as a solid.

Cis isomers: LCMS (m/z): 292.1 [M+H]+; Rt=0.95 min. Analytical HPLC: Rt=3.91 min.

¹H NMR (400 MHz, methanol-d4) δ [ppm]: 1.16 (d, J=7.04 Hz, 3H) 1.58-1.83 (m, 3H) 1.86-1.95 (m, 1H) 2.43 (tt, J=11.74, 4.30 Hz, 1H) 2.98 (t, J=12.91 Hz, 1H) 3.68 (s, 3H) 4.15-4.25 (m, 1H) 4.39-4.49 (m, 1H) 5.12 (s, 2H) 7.27-7.38 (m, 5H).

Trans isomers: LCMS (m/z): 292.1 [M+H]+; Rt=0.93 min. Analytical HPLC: Rt=3.75 min.

¹H NMR (400 MHz, methanol-d4) δ [ppm]: 1.11-1.23 (m, 3H) 1.38-1.47 (m, 1H) 1.76-2.06 (m, 3H) 2.66 (br. s., 1H) 3.19 (dd, J=13.89, 4.11 Hz, 1H) 3.58 (s, 3H) 4.33-4.46 (m, 2H) 5.02-5.08 (m, 1H) 5.10-5.19 (m, 1H) 7.27-7.39 (m, 5H)

### Step 3-a: Preparation of (3R,6S)-/(3S,6R)-1-(benzyloxycarbonyl)-6-methylpiperidine-3-carboxylic acid [cis isomers]

To a mixture of the cis isomers (3R,6S)-/(3S,6R)-6-methyl-piperidine-1,3-dicarboxylic acid 1-benzyl ester 3-methyl ester (1.55 g, 4.84 mmol) in MeOH (4.5 mL) and water (3 mL) was added 6N aqueous sodium hydroxide solution (1.5 mL, 9 mmol). The reaction mixture was stirred at 25° C. for 2 hrs and concentrated under reduced pressure to a volume of ˜2 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,6S)- and (3S,6R)-1-(benzyloxycarbonyl)-6-methylpiperidine-3-carboxylic acid (1.56 g) as a colorless oil, which was directly used in the next step without further purification. LCMS (m/z): 278.1 [M+H]+; Rt=0.79 min.

### Step 3-b: Preparation of (3R,6R)-/(3S,6S)-1-(benzyloxycarbonyl)-6-methylpiperidine-3-carboxylic acid [trans isomers]

To a mixture of the trans isomers (3R,6R)-/(3S,6S)-6-methyl-piperidine-1,3-dicarboxylic acid 1-benzyl ester 3-methyl ester (884 mg, 3.03 mmol) in MeOH (3 mL) and water (2 mL) was added 6N aqueous sodium hydroxide solution (1.0 mL, 6.0 mmol). The reaction mixture was stirred at 25° C. for 2 hrs and concentrated under reduced pressure to a volume of ˜2 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,6R)-/(3S,6S)-1-(benzyloxycarbonyl)-6-methylpiperidine-3-carboxylic acid (870 mg) as a white solid, which was directly used in the next step without further purification. LCMS (m/z): 278.1 [M+H]+; Rt=0.77 min

### Synthesis of 4-(tert-butoxycarbonyl)-1,4-oxazepane-6-carboxylic acid

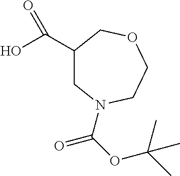

### Step 1: Preparation of tert-butyl 6-methylene-1,4-oxazepane-4-carboxylate

To sodium hydride (60 wt. % in mineral oil, 2.464 g, 61.6 mmol) in DMF (50 mL) was added 3-chloro-2-(chloromethyl)prop-1-ene (3.5 g, 28.0 mmol) at ˜5° C. (ice bath) and a solution of tert-butyl(2-hydroxyethyl)carbamate (4.51 g, 28.0 mmol) in tetrahydrofuran (50 mL). The reaction mixture was stirred at 20-30° C. for ˜2 hrs and concentrated under reduced pressure to remove tetrahydrofuran. The resulting mixture was poured into water and extracted with EtOAc. The combined organic extracts were washed with brine, dried over sodium sulfate, filtered off and concentrated under reduced pressure. The residue was purified by column chromatography [silica gel, 80 g, EtOAc/heptane=0/100 to 50/50] providing tert-butyl 6-methylene-1,4-oxazepane-4-carboxylate (4 g) as a colorless oil. ¹H NMR (400 MHz, chloroform-d) δ [ppm]: 1.46 (s, 9H) 3.33-3.62 (m, 2H) 3.62-3.82 (m, 2H) 4.09 (m, 2H) 4.16 (m, 2H) 4.99 (m, 2H).

### Step 2: Preparation of tert-butyl 6-(hydroxymethyl)-1,4-oxazepane-4-carboxylate

To a solution of tert-butyl 6-methylene-1,4-oxazepane-4-carboxylate (3.2 g, 15.0 mmol) in tetrahydrofuran (15 mL) was added borane tetrahydrofuran (1M solution in tetrahydrofuran, 13.50 mL) at 25° C. via a syringe. The colorless mixture was stirred at room temperature for 3 hrs. The reaction mixture was cooled to 0° C. and 3N aqueous sodium hydroxide solution (5 mL, 15.00 mmol) and aqueous hydrogen peroxide (˜30 wt. %, 2 mL, 19.6 mmol) were added sequentially. The obtained white cloudy mixture was stirred overnight and diluted with pentane. The separated organic layer was dried over potassium carbonate, filtered off and concentrated under reduced pressure. The residue was purified by column chromatography [silica gel, 40 g, EtOAc/heptane=0/100 to 50/50] providing tert-butyl 6-(hydroxymethyl)-1,4-oxazepane-4-carboxylate (2.6 g) as a colorless oil.

### Step 3: Preparation of tert-butyl 6-formyl-1,4-oxazepane-4-carboxylate

To a solution of tert-butyl 6-(hydroxymethyl)-1,4-oxazepane-4-carboxylate (0.9 g, 3.89 mmol) in (15 mL) was added Dess-Martin periodinane (1.650 g, 3.89 mmol) and the mixture was stirred at room temperature for ˜64 hrs. The reaction mixture was diluted with dichloromethane (60 mL) and washed with water, saturated aqueous sodium bicarbonate solution and brine. The organic layer was dried over sodium sulfate, filtered off and concentrated under reduced pressure providing crude tert-butyl 6-formyl-1,4-oxazepane-4-carboxylate (0.45 g) of nearly colorless oil, which was directly used in the next reaction.

### Step 4: Preparation of 4-(tert-butoxycarbonyl)-1,4-oxazepane-6-carboxylic acid

To a mixture of tert-butyl 6-formyl-1,4-oxazepane-4-carboxylate (0.45 g, 1.963 mmol) in tert-butanol (5 mL) was added sodium chlorite (0.231 g, 2.55 mmol) and sodium dihydrogen phosphate (0.306 g, 2.55 mmol) in water (1 mL) at 0° C. The mixture was allowed to warm to room temperature and stirred for about 16 hrs. The mixture was filtered and the filtrate was poured into water and extracted with EtOAc. The combined organic extracts were washed with brine, dried with sodium sulfate, filtered off and concentrated under reduced pressure providing 4-(tert-butoxycarbonyl)-1,4-oxazepane-6-carboxylic acid (0.73 g) as a colorless oil, which was directly used in the next step without further purification. LCMS (m/z): 190.1 [M+H, loss of t-Bu]+; Rt=0.60 min. ¹H NMR (400 MHz, chloroform-d) δ [ppm]: 1.38-1.57 (br. s, 9H) 2.92-3.24 (m, 1H) 3.28-3.44 (m, 1H) 3.47-4.19 (m, 7H).

### Synthesis of 1-(tert-butoxycarbonyl)azepane-3-carboxylic acid

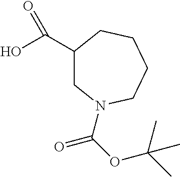

### Step 1: Preparation of ethyl 3-(allylamino)propanoate

To a solution of allyl amine (2.62 mL, 35.0 mmol) in EtOH (50 mL) was added ethyl acrylate (3.81 mL, 35.0 mmol) at 25° C. and the mixture was stirred under argon for ˜16 hrs. The mixture was concentrated under reduced pressure providing crude ethyl 3-(allylamino)propanoate (5.5 g) as an oil, which was used in the next step without further purification.

### Step 2: Preparation of ethyl 3-(allyl(tert-butoxycarbonyl)amino)propanoate

To a solution of ethyl 3-(allylamino)propanoate (5.50 g, 35.0 mmol) in dichloromethane (50 mL) was added sequentially diisopropylamine (6.11 mL, 35.0 mmol), DMAP (0.428 g, 3.50 mmol) and di-tert-butyl dicarbonate (8.13 mL, 35 mmol). The mixture was stirred at room temperature under argon for about 16 hrs. The reaction mixture was poured into water and extracted with dichloromethane. The organic extracts were combined, washed with brine, dried over sodium sulfate, filtered off and concentrated under reduced pressure providing ethyl 3-(allyl(tert-butoxycarbonyl)amino)propanoate (9.12 g) as a yellow oil, which was used in the next step without further purification. LCMS (m/z): 258.1 [M+H], 158.1 [M+H, loss of Boc group]+; Rt=0.95 min.

### Step 3: Preparation of ethyl 2-((allyl(tert-butoxycarbonyl)amino)methyl)pent-4-enoate

To a solution of ethyl 3-(allyl(tert-butoxycarbonyl)amino)propanoate (2 g, 7.77 mmol) in tetrahydrofuran (20 mL) was added lithium bis(trimethylsilyl)amide (8.55 mL, 8.55 mmol) slowly at −78° C. The mixture was stirred for 1 hr and allyl iodide (0.787 mL, 8.55 mmol) was added. The reaction mixture was allowed to warm slowly to room temperature and stirring was continued for 16 hrs. The reaction mixture was poured into water and extracted with EtOAc. The organic extracts were combined, washed with brine, dried with sodium sulfate, filtered off and concentrated under reduced pressure providing ethyl 2-((allyl(tert-butoxycarbonyl)amino)methyl)pent-4-enoate (2.15 g) as a brown oil, which was directly used in the next step without further purification. LCMS (m/z): 198.1 [M+H, loss of Boc group]+; Rt=1.11 min.

### Step 4: Preparation of 2,3,4,7-tetrahydro-azepine-1,3-dicarboxylic acid 1-tert-butyl ester 3-ethyl ester

To a solution of crude ethyl 2-((allyl(tert-butoxycarbonyl)amino)methyl)pent-4-enoate (2.15 g, 7.23 mmol) in dichloromethane (400 mL) under argon was added bis(tricyclohexylphosphine)benzylidine ruthenium(IV)chloride (Grubbs I catalyst; 0.605 g, 0.723 mmol). The reaction mixture was heated to reflux (45 to 65° C. oil bath temperature) for ˜5 hrs. The solvent was removed under reduced pressure and the residue was purified by column chromatography [silica gel, 80 g, EtOAc/heptane=0/100 to 30/70] providing 2,3,4,7-tetrahydro-azepine-1,3-dicarboxylic acid 1-tert-butyl ester 3-ethyl ester (1.84 g) as a black oil. LCMS (m/z): M+1=170.1 [M+H, loss of Boc group]+; Rt=0.96 min.

### Step 5: Preparation of azepane-1,3-dicarboxylic acid 1-tert-butyl ester 3-ethyl ester

To a solution of 2,3,4,7-tetrahydro-azepine-1,3-dicarboxylic acid 1-tert-butyl ester 3-ethyl ester (1.6 g, 5.94 mmol) in MeOH (40 mL) and tetrahydrofuran (10 mL) was added Pd/C (10 wt. %, 0.632 g). The mixture was stirred under hydrogen (balloon) for about 60 hrs. The reaction mixture was diluted with dichloromethane and filtered through celite pad. The filtrate was concentrated under reduced pressure and the residue was purified by column chromatography [silica gel, 80 g, EtOAc/heptane=0/100 to 20/80] providing azepane-1,3-dicarboxylic acid 1-tert-butyl ester 3-ethyl ester (0.6 g) as a brown oil.

### Step 6: Preparation of 1-(tert-butoxycarbonyl)azepane-3-carboxylic acid

To a solution of azepane-1,3-dicarboxylic acid 1-tert-butyl ester 3-ethyl ester (0.6 g, 2.211 mmol) in tetrahydrofuran (8 mL) was added 1N aqueous lithium hydroxide solution (2.65 mL, 2.65 mmol). The mixture was stirred at room temperature for 16 hrs and then was heated to 55° C. for 16 hrs. The reaction mixture was diluted with dichloromethane (10 mL) and extracted with 1N aqueous sodium hydroxide solution (2×20 mL). The aqueous extracts were acidified with 10% aqueous hydrochloride solution until pH˜5 and extracted with EtOAc. The organic extracts were washed with brine, dried with sodium sulfate, filtered off and concentrated under reduced pressure providing crude 1-(tert-butoxycarbonyl)azepane-3-carboxylic acid (0.4 g) as a colorless oil. ¹H NMR (400 MHz, chloroform-d) δ [ppm]: 1.36-1.52 (br. s, 9H) 1.52-2.10 (m, 6H) 2.65-2.98 (m, 1H) 3.04-3.72 (m, 3H) 3.72-3.97 (m, 1H).

### Synthesis of 1-benzyl-6,6-dimethylpiperidine-3-carboxylic acid

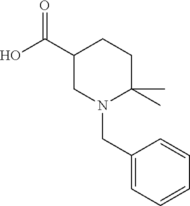

### Step 1: Preparation of 1-phenyl-N-(propan-2-ylidene)methanamine

To a well mixed mixture of acetone (4.65 g, 80 mmol) and basic alumina (15 g) was added a pre-mixed mixture of benzylamine (8.57 g, 80 mmol) and basic alumina (20 g) in portions under gentle shaking. The resultant mixture was hand shaked for 5 min and let stand for ˜1.5 days. The mixture was extracted with dichloromethane (3×15 mL). The combined organic layers were concentrated under reduced pressure and were further dried in high vacuo for 1 day at 60° C. providing crude 1-phenyl-N-(propan-2-ylidene)methanamine (6.3 g) as a light yellow oil, which was directly used in the next step. ¹H NMR (300 MHz, chloroform-d) δ [ppm]: 1.93 (s, 3H) 2.09 (s, 3H) 4.46 (s, 2 H) 7.20-7.41 (m, 5H).

### Step 2: Preparation of N-benzyl-2-methylpent-4-en-2-amine

To a solution of 1-phenyl-N-(propan-2-ylidene)methanamine (1.472 g, 10 mmol) in diethylether (20 mL) was added slowly allymagnesium bromide (1 m solution in tetrahydrofuran, 22 mL) at 0° C. The reaction mixture was stirred at 0° C. for 1 hr and at room temperature for 3 hrs. The mixture was diluted with saturated aqueous ammonium chloride solution and the separated aqueous layer was extracted with diethylether. The combined organic layers were dried over sodium sulfate, filtered off and concentrated under reduced pressure providing crude N-benzyl-2-methylpent-4-en-2-amine (1.75 g), which was directly used at next step without further purification. ¹H NMR (300 MHz, chloroform-d) δ [ppm]: 1.14-1.31 (m, 6H) 2.20-2.40 (m, 2H) 3.71-3.77 (m, 4H) 5.03-5.15 (m, 2H) 5.80-5.90 (m, 1H) 7.20-7.36 (m, 5H).

### Step 3: Preparation of ethyl 2-((benzyl(2-methylpent-4-en-2-yl)amino)methyl)acrylate

To a solution of N-benzyl-2-methylpent-4-en-2-amine (284 mg, 1.5 mmol) in acetonitrile (4 mL) was added powdered potassium carbonate (498 mg, 2.4 mmol) and ethyl 2-(bromomethyl)acrylate (319 mg, 1.65 mmol) and the mixture was stirred at room temperature overnight. The reaction mixture was filtered and the filterate was concentrated under reduced pressure. The residue was purified by column chromatography [silica gel, 24 g, EtOAc/heptane=0/100 to 25/75] providing ethyl 2-((benzyl(2-methylpent-4-en-2-yl)amino)methyl)acrylate (194 mg) as a clear liquid. LCMS (m/z): 302.2 [M+H]+; Rt=0.73 min.

### Step 4: Preparation of ethyl 1-benzyl-6,6-dimethyl-1,2,5,6-tetrahydropyridine-3-carboxylate

To a solution of ethyl 2-((benzyl(2-methylpent-4-en-2-yl)amino)methyl)acrylate (194 mg, 0.644 mmol) in toluene (6.5 mL) under nitrogen atmosphere was added p-toluenesulfonic acid monohydrate (135 mg, 0.708 mmol). The mixture was heated to 50° C. for 30 min, (1,3-bis(2,4,6-trimethylphenyl)-2-(imidazolidinylidene)(dichlorophenylmethylene)-(tricyclohexylphosphine)ruthenium (2nd generation Grubbs catalyst, 27.3 mg) was added and heated was continued at 55° C. for 5 hrs. The mixture was allowed to cool to room temperature, diluted with saturated aqueous sodium carbonate solution (2 mL) and filtered through a pad of celite. The separated organic phase was dried over sodium sulfate, filtered off and concentrated under reduced pressure. The residue was purified by column chromatography [silica gel, 24 g, EtOAc/heptane=10/90 to 25/75] providing ethyl 1-benzyl-6,6-dimethyl-1,2,5,6-tetrahydropyridine-3-carboxylate (117 mg) as a clear liquid. LCMS (m/z): 274.1 [M+H]+; Rt=0.58 min.

### Step 5: Preparation of ethyl 1-benzyl-6,6-dimethylpiperidine-3-carboxylate

To a solution of 1-benzyl-6,6-dimethyl-1,2,5,6-tetrahydropyridine-3-carboxylate (117 mg, 0.428 mmol) in MeOH (5 mL) was added magnesium (turnings, 41.6 mg, 1.712 mmol) and the mixture was vigorously stirred at 33° C. for 5 hrs. The mixture was partitioned between saturated aqueous ammonium chloride solution (20 mL) and diethylether (20 mL). The separated aqueous layer was extracted with diethylether (3×10 mL) and the combined organic layers were dried over sodium sulfate, filtered off and concentrated under reduced pressure providing crude ethyl 1-benzyl-6,6-dimethylpiperidine-3-carboxylate (115 mg) as a light yellow liquid, which was directly used at next step without further purification. LCMS (m/z): 276.2 [M+H]+; Rt=0.59 min.

### Step 6: Preparation of 1-benzyl-6,6-dimethylpiperidine-3-carboxylic acid

A mixture of 1-benzyl-6,6-dimethyl-1,2,5,6-tetrahydropyridine-3-carboxylate (118 mg, 0.428 mmol) and lithium hydroxide (102 mg, 4.28 mmol) in tetrahydrofuran (1 mL), MeOH (1 mL) and water (0.5 mL) was stirred at room temperature overnight. The mixture was acidified with 1N aqueous hydrochloride solution until pH˜5-6 and extracted with EtOAc (5×20 mL). The combined organic layers were dried over sodium sulfate, filtered off and concentrated under reduced pressure providing crude 1-benzyl-6,6-dimethylpiperidine-3-carboxylic acid (55 mg), which was directly used in the next step without further purification. LCMS (m/z): 248.2 [M+H]+; Rt=0.38 min.

### Synthesis of 1-(tert-butoxycarbonyl)-6,6-dimethylpiperidine-3-carboxylic acid

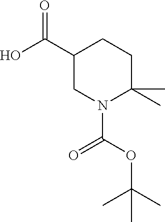

### Step 1: Preparation of methyl 6,6-dimethylpiperidine-3-carboxylate

A mixture of methyl 1-benzyl-6,6-dimethylpiperidine-3-carboxylate (55 mg, 0.210 mmol), ammonium formate (66.3 mg, 1.052 mmol) and Pd/C (10 wt. %, water 50 wt. %, 6 mg) in MeOH (1 mL) was stirred at 70° C. for 30 min. The mixture was allowed to cool to room temperature filtered off to remove Pd/C and solids. The filterate was concentrated in high vacuo providing crude methyl 6,6-dimethylpiperidine-3-carboxylate (36 mg) as a light yellow liquid, which was directly used without further purification. LCMS (m/z): 171.4 [M+H]+; Rt=0.21 min.

### Step 2: Preparation of 6,6-dimethyl-piperidine-1,3-dicarboxylic acid 1-tert-butyl ester 3-methyl ester

To a mixture of methyl 6,6-dimethylpiperidine-3-carboxylate (36.0 mg, 0.21 mmol) and triethylamine (0.088 mL, 0.630 mmol) in tetrahydrofuran (1.5 mL) was added BOC-anhydride (0.059 mL, 0.252 mmol). The reaction mixture was stirred at 35° C. overnight and concentrated under reduced pressure providing crude 6,6-dimethyl-piperidine-1,3-dicarboxylic acid 1-tert-butyl ester 3-methyl ester (61 mg), which was directly used in the next step without further purification.

### Step 3: Preparation of 1-(tert-butoxycarbonyl)-6,6-dimethylpiperidine-3-carboxylic acid

A mixture of 6,6-dimethyl-piperidine-1,3-dicarboxylic acid 1-tert-butyl ester 3-methyl ester (60 mg, 0.221 mmol) and lithium hydroxide (5.30 mg, 0.221 mmol) in tetrahydrofuran (1 mL), MeOH (1 mL) and water (0.5 mL) was stirred overnight at room temperature. The mixture was concentrated under reduced pressure to remove most of the organic solvents. The residue was acidified with 1N aqueous hydrochloride solution until pH˜5 and extracted with EtOAc (2×20 mL). The combined organic layers were dried over sodium sulfate, filtered off and concentrated under reduced pressure providing crude 1-(tert-butoxycarbonyl)-6,6-dimethylpiperidine-3-carboxylic acid (21 mg), which was directly used in the next step without further purification.

### Synthesis of 1-(benzyloxycarbonyl)-6-(trifluoromethyl)piperidine-3-carboxylic acid

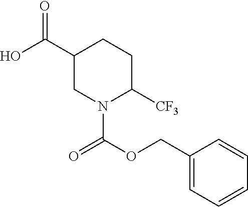

### Step 1: Preparation of ethyl 6-(trifluoromethyl)piperidine-3-carboxylate (mixture of cis and trans isomers)

A mixture of ethyl 6-(trifluoromethyl)nicotinate (2.2 g, 10 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 24 hrs. The reaction mixture was filtered through a pad of celites and washed with MeOH (150 mL). The filtrate was concentrated under reduced pressure providing crude ethyl 6-(trifluoromethyl)piperidine-3-carboxylate (776 mg; mixture of cis and trans isomers) as a colorless oil, which was directly used in the next step without further purification. LCMS (m/z): 226.1 [M+H]+; Rt=0.36 min.

### Step 2: Preparation of 6-trifluoromethyl-piperidine-1,3-dicarboxylic acid 1-benzyl ester 3-ethyl ester [mixture of 4 isomers]

To a mixture of crude ethyl 6-(trifluoromethyl)piperidine-3-carboxylate (766 mg, 3.4 mmol) aqueous sodium carbonate solution (10 wt. %, 5 mL) in tetrahydrofuran (15 mL) was added slowly benzylchloroformate (0.583 mL, 4.08 mmol). The reaction mixture was stirred at 25° C. for 24 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 24 g, EtOAc/heptane=0/100 to 30/70] providing a mixture of the cis and trans isomers of 6-trifluoromethyl-piperidine-1,3-dicarboxylic acid 1-benzyl ester 3-ethyl ester (826 mg) as an oil. LCMS (m/z): 316.1 [M+H]+; Rt=1.07 min.

### Step 3: Preparation of 1-(benzyloxycarbonyl)-6-(trifluoromethyl)piperidine-3-carboxylic acid [mixture of 4 isomers]

To 1-benzyl 6-trifluoromethyl-piperidine-1,3-dicarboxylic acid 1-benzyl ester 3-ethyl ester (823 mg, 2.38 mmol) in MeOH (1.8 mL) and water (1.2 mL) was added 6N aqueous sodium hydroxide solution (0.6 mL, 3.6 mmol). The resulting reaction mixture was stirred at 25° C. for 1.5 hrs and concentrated under reduced pressure to a volume of ˜0.5 mL. The mixture was acidified with 1N hydrochloride solution until pH˜4, diluted with EtOAc and stirred for 10 min. The separated organic layer was washed with brine solution, dried over sodium sulfate, filtered off and concentrated under reduced pressure providing 1-(benzyloxycarbonyl)-6-(trifluoromethyl)piperidine-3-carboxylic acid (782 mg, mixture of 4 isomers) as a colorless oil, which was directly used in the next step without further purification. LCMS (m/z): 332.0 [M+H]+; Rt=0.90 min.

### Synthesis of (3R,6R)-/(3S,6S)-1-(benzyloxycarbonyl)-6-ethylpiperidine-3-carboxylic acid and (3R,6S)-/(3R,6S)-1-(benzyloxycarbonyl)-6-ethylpiperidine-3-carboxylic acid

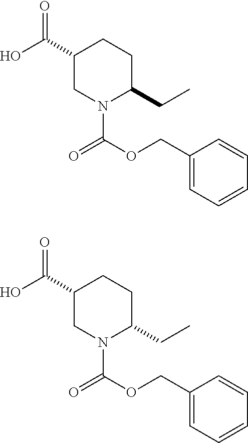

### Step 1: Preparation of methyl 6-ethylnicotinate

To a solution of methyl 6-chloronicotinate (5.0 g, 29.1 mmol), ferric acetylacetonate (1.0 g, 2.83 mmol) in tetrahydrofuran (160 mL) and NMP (1 mL) was added slowly a solution of ethylmagnesium bromide (1M in tetrahydrofuran, 1.09 mL, 7.27 mmol). The reaction mixture was stirred at 25° C. for 3 hrs. The reaction mixture was diluted with saturated aqueous ammonium chloride solution and stirred for additional 30 min. The mixture was diluted with EtOAc, the separated organic layer was washed with saturated aqueous ammonium chloride solution, water and brine. The organic phase was dried over sodium sulfate, filtered off and concentrated under reduced pressure. The residue was purified by column chromatography [silica gel, 80 g, EtOAc/heptane=0/100 to 30/70] providing methyl 6-ethylnicotinate (2.48 g) as an oil. LCMS (m/z): 166.1 [M+H]+; Rt=0.32 min.

### Step 2: Preparation of methyl 6-ethylpiperidine-3-carboxylate (mixture of cis and trans isomers)

A mixture of methyl 6-ethylnicotinate (2.48 g, 15 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 16 hrs. The reaction mixture was filtered through a pad of celites and washed with MeOH (150 mL). The filtrate was concentrated under reduced pressure providing crude methyl 6-ethylpiperidine-3-carboxylate (4.45 g; mixture of cis and trans isomers) as a colorless oil, which was directly used in the next step without further purification. LCMS (m/z): 172.1 [M+H]+; Rt=0.31 min.

### Step 3: Preparation of (3R,6S)-/(3S,6R)-6-ethyl-piperidine-1,3-dicarboxylic acid 1-benzyl ester 3-methyl ester [cis isomers] and (3R,6R)-/(3S,6S)-6-ethyl-piperidine-1,3-dicarboxylic acid 1-benzyl ester 3-methyl ester [trans isomers]

To a mixture of crude methyl 6-ethylpiperidine-3-carboxylate (4.5 g, 15 mmol) aqueous sodium carbonate solution (10 wt. %, 30 mL) in tetrahydrofuran (60 mL) was added slowly benzylchloroformate (2.14 mL, 15 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 30/70] providing a mixture of the cis isomers (3R,6S)-/(3S,6R)-6-ethyl-piperidine-1,3-dicarboxylic acid 1-benzyl ester 3-methyl ester (3.03 g) as a colorless oil and a mixture of the trans isomers (3R,6R)-/(3S,6S)-6-ethyl-piperidine-1,3-di carboxylic acid 1-benzyl ester 3-methyl ester (1.23 g) as a solid.

Cis isomers: LCMS (m/z): 306.1 [M+H]+; Rt=1.01 min. Analytical HPLC: Rt=4.15 min.

¹H NMR (400 MHz, methanol-d4) δ [ppm]: 0.83 (t, J=6.85 Hz, 3H) 1.49 (d, J=5.87 Hz, 1H) 1.66-1.76 (m, 4H) 1.85-1.93 (m, 1H) 2.38-2.49 (m, J=11.79, 11.79, 4.21, 3.91 Hz, 1H) 2.90 (d, J=1.96 Hz, 1H) 3.67 (s, 3H) 4.16-4.29 (m, 2H) 5.12 (br. s., 2H) 7.28-7.40 (m, 5H).

Trans isomers: LCMS (m/z): 306.1 [M+H]+; Rt=0.98 min. Analytical HPLC: Rt=4.01 min.

¹H NMR (400 MHz, methanol-d4) δ [ppm]: 0.83 (t, J=7.43 Hz, 3H) 1.43-1.57 (m, 2 H) 1.71-1.93 (m, 3H) 1.94-2.02 (m, 1H) 2.64 (br. s., 1H) 3.11 (dd, J=14.09, 3.91 Hz, 1H) 3.49-3.69 (m, 3H) 4.11-4.20 (m, 1H) 4.45 (d, J=13.69 Hz, 1H) 5.03-5.19 (m, 2H) 7.19-7.40 (m, 5H).

### Step 3-a: Preparation of (3R,6R)-/(3S,6S)-1-(benzyloxycarbonyl)-5-ethylpiperidine-3-carboxylic acid [trans isomers]

To a mixture of trans isomers (3R,6R)-/(3S,6S)-1-benzyl 3-methyl 6-ethylpiperidine-1,3-dicarboxylate (1.23 g, 3.1 mmol) in MeOH (3 mL) and water (2 mL) was added 6N aqueous sodium hydroxide solution (1.0 mL, 6 mmol). The reaction mixture was stirred at 25° C. for 2.5 hrs and concentrated under reduced pressure to a volume of ˜2 mL. The mixture was acidified with 1N aqueous hydrochloride solution until pH˜4, diluted with EtOAc and stirred for 10 min. The separated organic layer was washed with brine, dried over sodium sulfate, filtered off and concentrated under reduced pressure providing a mixture of crude (3R,6R)-/(3S,6S)-1-(benzyloxycarbonyl)-6-ethylpiperidine-3-carboxylic acid (1.02 g) as a white solid, which was directly used in the next step without further purification. LCMS (m/z): 292.2 [M+H]+; Rt=0.85 min.

### Step 3-b: Preparation of (3R,6S)-/(3S,6R)-1-(benzyloxycarbonyl)-6-ethylpiperidine-3-carboxylic acid [cis isomers]

To a mixture of cis isomers (3R,6S)-/(3S,6R)-1-benzyl 3-methyl 6-ethylpiperidine-1,3-dicarboxylate (0.92 g, 3.0 mmol) in MeOH (3 mL) and water (2 mL) was added 6N aqueous sodium hydroxide solution (1.0 mL, 6 mmol). The reaction mixture was stirred at 25° C. for 1.5 hrs and concentrated under reduced pressure to a volume of ˜2 mL. The mixture was acidified with 1N aqueous hydrochloride solution until pH˜4, diluted with EtOAc and stirred for 10 min. The separated organic layer was washed with brine, dried over sodium sulfate, filtered off and concentrated under reduced pressure providing a mixture of crude (3R,6S)-/(3S,6R)-1-(benzyloxycarbonyl)-6-ethylpiperidine-3-carboxylic acid (0.91 g) as an oil, which was directly used in the next step without further purification. LCMS (m/z): 292.1 [M+H]+; Rt=0.87 min.

### Synthesis of (3R,6S)-/(3S,6R)-1-(benzyloxycarbonyl)-6-(methoxymethyl)piperidine-3-carboxylic acid

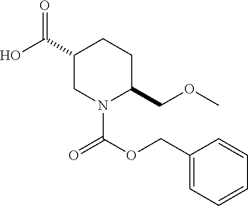

### Step 1: Preparation of methyl 6-(hydroxymethyl)nicotinate

To a mixture of dimethylpyridine-2,5-dicarboxylate (3.08 g, 15.78 mmol) and calcium chloride (7.01 g, 63.1 mmol) in tetrahydrofuran (33 mL) and EtOH (67 mL) was added sodium borohydride (1.493 g, 39.5 mmol) in portions at 0° C. The reaction mixture was stirred at 0° C. for 12 hrs. The mixture was poured into ice/water, was diluted with dichloromethane (400 mL) and stirred vigorously for 15 minutes. The separated organic layer was dried over magnesium sulfate, filtered off and concentrated under reduced pressure providing methyl 6-(hydroxymethyl)nicotinate (1.2 g) as an off white solid, which was directly used in the next step without further purification. LCMS (m/z): 168.0 [M+H]+; Rt=0.26 min

### Step 2: Preparation of methyl 6-(chloromethyl)nicotinate

A mixture of methyl 6-(hydroxymethyl)nicotinate (250 mg, 1.496 mmol) and thionyl chloride (1 mL, 13.70 mmol) in dichloromethane (2 mL) was stirred at 45° C. for 3 hrs and concentrated under reduced pressure. The residue was taken up in dichloromethane (25 mL), sonicated and concentrated under reduced pressure. This was repeated three times and the residue was dried in high vacuo providing of methyl 6-(chloromethyl)nicotinate (266 mg), which was used in the next reaction without further purification. LCMS (m/z): 186.0 [M+H]+; Rt=0.63 min.

### Step 3: Preparation of methyl 6-(methoxymethyl)nicotinate

To a solution of methyl 6-(chloromethyl)nicotinate (250 mg, 1.347 mmol) in MeOH (2 mL) was added sodium methoxide (25 wt. % in MeOH; 1 mL). The mixture was heated at 75° C. for 30 min and concentrated under reduced pressure. The residue was dissolved in EtOAc and the organic layer was washed saturated aqueous sodium bicarbonate solution (3×), dried over magnesium sulfate, filtered off and concentrated under reduced pressure. The residue was purified by column chromatography [silica gel, 12 g, EtOAc/heptane=0/100 to 70/30] providing methyl 6-(methoxymethyl)nicotinate (129 mg). LCMS (m/z): 182.0 [M+H]+; Rt=0.43 min.

### Step 4: Preparation of methyl 6-(methoxymethyl)piperidine-3-carboxylate (mixture of cis and trans isomers)

A mixture of methyl 6-(methoxymethyl)nicotinate (250 mg, 1.380 mmol) and platinum(IV)oxide (100 mg, 0.440 mmol) in acetic acid (10 mL) was stirred in a steel bomb under hydrogen atmosphere (200 psi) at 25° C. for 12 hrs. The reaction mixture was filtered through a pad of celites and washed with dichloromethane (50 mL). The filtrate was concentrated under reduced pressure providing crude methyl 6-(methoxymethyl)piperidine-3-carboxylate (266 mg; mixture of cis and trans isomers) as a colorless oil, which was directly used in the next step without further purification. LCMS (m/z): 188.1 [M+H]+; Rt=0.30 min.

### Step 5: Preparation of (3S,6R)-/(3R,6S)-6-methoxymethyl-piperidine-1,3-dicarboxylic acid 1-benzyl ester 3-methyl ester [trans isomers] and (3R,6R)-/(3S,6S)-6-methoxymethyl-piperidine-1,3-dicarboxylic acid 1-benzyl ester 3-methyl ester [cis isomers]

To a mixture of methyl 6-(methoxymethyl)piperidine-3-carboxylate (260 mg, 1.389 mmol) and aqueous sodium carbonate solution (10 wt. %; ˜4 mL) in tetrahydrofuran (4 mL) was added slowly benzylchloroformate (0.297 mL, 2.083 mmol). The reaction mixture was stirred at 25° C. for 1 hr. The mixture was diluted with EtOAc and stirred for additional 10 min. The separated organic layer was dried over magnesium sulfate, filtered off and concentrated under reduced pressure. The residue was purified by column chromatography [silica gel, 12 g, EtOAc/heptane=0/100 to 70/30] providing a mixture of the trans isomers (3S,6R)-/(3R,6S)-6-methoxymethyl-piperidine-1,3-dicarboxylic acid 1-benzyl ester 3-methyl ester (256 mg) and a mixture of the cis isomers (3R,6R)-/(3S,6S)-6-methoxymethyl-piperidine-1,3-dicarboxylic acid 1-benzyl ester 3-methyl ester (200 mg).

Cis isomers: LCMS (m/z): 322.1 [M+H]+; Rt=0.89 min. Analytical HPLC: Rt=4.20 min.

Trans isomers: LCMS (m/z): 322.1 [M+H]+; Rt=0.86 min. Analytical HPLC: Rt=3.98 min.

### Step 6-a: Preparation of (3S,6R)-/(3R,6S)-1-(benzyloxycarbonyl)-6-(methoxymethyl)piperidine-3-carboxylic acid [trans isomers]

To 1-benzyl 3-methyl 6-(methoxymethyl)piperidine-1,3-dicarboxylate (40 mg, 0.124 mmol) in MeOH (3 mL) was added 1N aqueous sodium hydroxide solution (3 mL). The reaction mixture was stirred at 25° C. for 12 hrs and concentrated under reduced pressure to a volume of ˜2 mL. The mixture was acidified with 12N hydrochloride until pH˜4, diluted with EtOAc and stirred for 10 min. The separated organic layer was dried over magnesium sulfate, filtered off and concentrated under reduced pressure providing a mixture of (3S,6R)-/(3R,6S)-1-(benzyloxycarbonyl)-6-(methoxymethyl)piperidine-3-carboxylic acid (35 mg) as a colorless oil, which was directly used in the next step without further purification. LCMS (m/z): 308.1 [M+H]+; Rt=0.73 min.

### Synthesis of (3S,4R)-1-(benzyloxycarbonyl)-4-isopropoxypyrrolidine-3-carboxylic acid

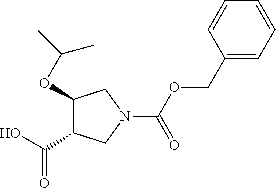

### Step 1: Preparation of (3R,4S)-benzyl 3-isopropoxy-4-vinylpyrrolidine-1-carboxylate

To a solution of (3R,4S)-benzyl 3-hydroxy-4-vinylpyrrolidine-1-carboxylate (3.0 g, 12.13 mmol) in acetonitrile (30 mL) was added 2-iodopropane (20.6 g, 121 mmol) and silver(I)oxide (8.43 g, 36.4 mmol). The mixture was stirred at room temperature for 18 hrs. The solid was filtered off and the filtrate was concentrated under reduced pressure. The residue was purified by column chromatography [silica gel] providing (3R,4S)-benzyl 3-isopropoxy-4-vinylpyrrolidine-1-carboxylate (870 mg). LCMS (m/z): 290.0 [M+H]+; Rt=1.03 min.

### Step 2: Preparation of (3S,4R)-1-(benzyloxycarbonyl)-4-isopropoxypyrrolidine-3-carboxylic acid

A mixture of (3R,4S)-benzyl 3-isopropoxy-4-vinylpyrrolidine-1-carboxylate (550 mg, 1.90 mmol), ruthenium trichloride (496 mg, 1.90 mmol) and sodium periodate (1.63 g, 7.60 mmol) in carbontetrachloride (10 mL), water (10 mL) and acetonitrile (10 mL) were stirred at room temperature overnight. The reaction mixture was diluted with dichloromethane (200 mL) and water (200 mL). The mixture was filtered off and the separated aqueous layer was washed with dichloromethane (2×). All organic layers were combined, dried over sodium sulfate, filtered off and concentrated under reduced pressure. The residue was purified by column chromatography [silica gel, EtOAc/heptane=0/100 to 90/10] providing (3S,4R)-1-(benzyloxycarbonyl)-4-isopropoxypyrrolidine-3-carboxylic acid (350 mg). LCMS (m/z): 308.0 [M+H]+; Rt=0.82 min.

### Synthesis of (3R,5S)-1-(tert-butoxycarbonyl)-5-((2-methoxyethoxy)methyl)pyrrolidine-3-carboxylic acid

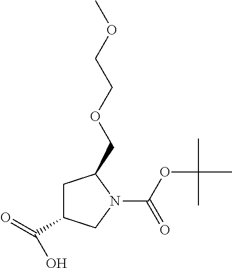

### Step 1: Preparation of (2S,4S)-4-(tert-butyl-diphenyl-silanyloxy)-pyrrolidine-1,2-dicarboxylic acid 1-tert-butyl ester 2-methyl ester

To a solution of (2S,4S)-4-hydroxy-pyrrolidine-1,2-dicarboxylic acid 1-tert-butyl ester 2-methyl ester (2.54 g, 10.25 mmol) in DCM (20 mL) was added the imidazole (1.187 g, 17.43 mmol) followed by tert-butylchlorodiphenylsilane (2.90 mL, 11.28 mmol) at room temperature and the reaction mixture was stirred for 18 hrs. The reaction mixture was filtered and the filtrate was washed with water and brine, dried over sodium sulfate, filtered off and concentrated under reduced pressure providing (2S,4S)-4-(tert-butyl-diphenyl-silanyloxy)-pyrrolidine-1,2-dicarboxylic acid 1-tert-butyl ester 2-methyl ester (4.9 g, 10.09 mmol, 98% yield). LCMS (m/z): 506.2 [M+H]+; Rt=1.46 min.

### Step 2: Preparation of (2S,4S)-tert-butyl 4-(tert-butyldiphenylsilyloxy)-2-(hydroxymethyl)pyrrolidine-1-carboxylate

To a solution of (2S,4S)-4-(tert-butyl-diphenyl-silanyloxy)-pyrrolidine-1,2-dicarboxylic acid 1-tert-butyl ester 2-methyl ester (5.6 g, 11.58 mmol) in tetrahydrofuran (50 mL) was added sodium borohydride (0.876 g, 23.16 mmol) and the mixture was stirred at 70° C. for 4 hrs. The reaction mixture was allowed to cool to room temperature and was diluted with EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2S,4S)-tert-butyl 4-(tert-butyldiphenylsilyloxy)-2-(hydroxymethyl)pyrrolidine-1-carboxylate (3.9 g). LCMS (m/z): 456.2 [M+H]+; Rt=1.30 min.

### Step 3: Preparation of (2S,4S)-tert-butyl 4-(tert-butyldiphenylsilyloxy)-2-((2-methoxyethoxy)methyl)pyrrolidine-1-carboxylate

To a solution of (2S,4S)-tert-butyl 4-(tert-butyldiphenylsilyloxy)-2-(hydroxymethyl)pyrrolidine-1-carboxylate (1.3 g, 2.86 mmol) in tetrahydrofuran (10 mL) was added carefully sodium hydride (60 wt. % in mineral oil, 142 mg, 3.42 mmol) and the mixture was stirred at 25° C. for 1 hr. To the mixture was added bromo ethyl methyl ether (0.714 g, 5.14 mmol) and stirring was continued at 25° C. for 18 hrs. The reaction mixture was diluted with EtOAc, washed with water, saturated aqueous sodium bicarbonate solution and brine and concentrated under reduced pressure. The residue was purified by column chromatography [silica gel] providing (2S,4S)-tert-butyl 4-(tert-butyldiphenylsilyloxy)-2-((2-methoxyethoxy)methyl)pyrrolidine-1-carboxylate (800 mg). LCMS (m/z): 514.2 [M+H]+; Rt=1.41 min.

### Step 4: Preparation of (2S,4S)-tert-butyl 4-hydroxy-2-((2-methoxyethoxy)methyl)-pyrrolidine-1-carboxylate

To a solution of (2S,4S)-tert-butyl 4-(tert-butyldiphenylsilyloxy)-2-((2-methoxyethoxy)methyl)pyrrolidine-1-carboxylate (310 mg, 0.603 mmol) in tetrahydrofuran (5 mL) was added tetrabutylammonium fluoride (316 mg, 1.207 mmol) and the mixture was stirred at 25° C. for 2 hrs. The reaction mixture was diluted with EtOAc (100 mL) and washed with water, brine, dried over sodium sulfate, filtered off and concentrated under reduced pressure. The residue was purified by column chromatography [silica gel, 24 g, EtOAc/heptane=0/100 to 50/50] providing (2S,4S)-tert-butyl 4-hydroxy-2-((2-methoxyethoxy)methyl)pyrrolidine-1-carboxylate (140 mg). LCMS (m/z): 298.1 [M+Na]+; Rt=0.67 min.

### Step 5: Preparation of (2S,4S)-tert-butyl 2-((2-methoxyethoxy)methyl)-4-(tosyloxy)pyrrolidine-1-carboxylate

A mixture of (2S,4S)-tert-butyl 4-hydroxy-2-((2-methoxyethoxy)methyl)pyrrolidine-1-carboxylate (140 mg, 0.508 mmol) and tosyl chloride (291 mg, 1.525 mmol) in pyridine (5 mL) was stirred at 25° C. for 18 hrs. The reaction mixture was diluted with EtOAc (50 mL), washed with water (2×) and brine. The organic layer was dried over sodium sulfate, filtered off and concentrated under reduced pressure. The residue was dissolved in dichloromethane (2 mL) and was purified by column chromatography [silica gel] providing (2S,4S)-tert-butyl 24(2-methoxyethoxy)methyl)-4-(tosyloxy)pyrrolidine-1-carboxylate (180 mg, LCMS (m/z): 430.1 [M+H]+; Rt=1.06 min.

### Step 6: Preparation of (2S,4R)-tert-butyl 4-cyano-2-((2-methoxyethoxy)methyl)-pyrrolidine-1-carboxylate

To a solution of 2S,4S)-tert-butyl 2-((2-methoxyethoxy)methyl)-4-(tosyloxy)pyrrolidine-1-carboxylate (180 mg, 0.419 mmol) in DMF (2 mL) was added tetrabutylammonium cyanide (343 mg, 1.26 mmol) and the mixture was stirred at 60° C. for 18 hrs. The reaction mixture was diluted with EtOAc (50 mL) and washed with water and brine. The organic layer was dried over sodium sulfate, filtered off and concentrated under reduced pressure. The residue was purified by column chromatography [silica gel] providing (2S,4R)-tert-butyl 4-cyano-2-((2-methoxyethoxy)methyl)pyrrolidine-1-carboxylate (123 mg). LCMS (m/z): 285.1 [M+H]+; Rt=0.82 min.

### Step 7: Preparation of (3R,5S)-1-(tert-butoxycarbonyl)-5-((2-methoxyethoxy)methyl)-pyrrolidine-3-carboxylic acid

A mixture of (2S,4R)-tert-butyl 4-cyano-2-((2-methoxyethoxy)methyl)pyrrolidine-1-carboxylate (123 mg, 0.433 mmol), 6N aqueous sodium hydroxide solution (2 mL, 12 mmol) and EtOH (2 mL) in a closed vial was stirred at 85° C. for 3 hrs. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure. The residue was purified by column chromatography [silica gel]providing (3R,5S)-1-(tert-butoxycarbonyl)-5-((2-methoxyethoxy)methyl)pyrrolidine-3-carboxylic acid (29 mg). LCMS (m/z): 326.0 [M+Na]+; Rt=0.69 min.

### Synthesis of (3R,5S)-/(3S,5R)-1-(benzyloxycarbonyl)-5-methoxypiperidine-3-carboxylic acid and (3R,5R)-/(3S,5S)-1-(benzyloxycarbonyl)-5-methoxypiperidine-3-carboxylic acid

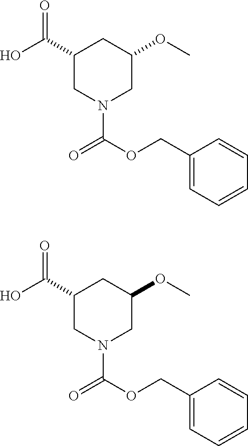

### Step 1: Preparation of methyl 5-methoxypiperidine-3-carboxylate (mixture of cis and trans isomers)

A mixture of methyl 5-methoxynicotinate (1 g, 5.98 mmol), Pd/C (10 wt. %, 90 mg) and platinum(IV)oxide (135 mg, 0.595 mmol) in acetic acid (18 mL) was stirred in a steel bomb under hydrogen atmosphere (200 psi) at 25° C. for 6 hrs. The reaction mixture was filtered through a Celite pad, and washed with MeOH (100 mL). The filtrate was concentrated under reduced pressure providing crude methyl 5-methoxypiperidine-3-carboxylate (1.53 g; mixture of cis and trans isomers) as a colorless oil, which was directly used in the next step without further purification. LCMS (m/z): 174.1 [M+H]+; Rt=0.26 min.

### Step 2: Preparation of (3R,5S)-/(3S,5R)-5-methoxy-piperidine-1,3-dicarboxylic acid 1-benzyl ester 3-methyl ester [cis isomers] and (3R,5R)-/(3S,5S)-5-methoxy-piperidine-1,3-dicarboxylic acid 1-benzyl ester 3-methyl ester [trans isomers]

To a mixture of crude methyl 5-methoxypiperidine-3-carboxylate (1.5 g, 6.06 mmol) aqueous sodium carbonate solution (10 wt. %, 12 mL) in tetrahydrofuran (38 mL) was added slowly benzylchloroformate (1.09 mL, 7.27 mmol). The reaction mixture was stirred at 25° C. for 90 min. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 50/50] providing a mixture of the cis isomers (3R,5S)-/(3S,5R)-5-methoxy-piperidine-1,3-dicarboxylic acid 1-benzyl ester 3-methyl ester (441 mg) as colorless oil and a mixture of the cis/trans isomers 5-methoxy-piperidine-1,3-dicarboxylic acid 1-benzyl ester 3-methyl ester (596 mg) as colorless oil.

Cis isomers: LCMS (m/z): 308.1 [M+H]+; Rt=0.89 min. Analytical HPLC: Rt=3.510 min.

Cis/Trans isomers: LCMS (m/z): 308.0 [M+H]+; Rt=0.83 min. Analytical HPLC: Rt=3.516 min.

### Step 3-a: Preparation of (3R,5S)-/(3S,5R)-1-(benzyloxycarbonyl)-5-methoxypiperidine-3-carboxylic acid [cis isomers]

To a mixture of the cis isomers (3R,5S)-/(3S,5R)-5-methoxy-piperidine-1,3-dicarboxylic acid 1-benzyl ester 3-methyl ester (440 mg, 1.43 mmol) in MeOH (1.44 mL) and water (0.96 mL) was added 6N aqueous sodium hydroxide solution (0.48 mL, 2.88 mmol). The reaction mixture was stirred at 25° C. for 1 hr and concentrated under reduced pressure to a volume of ˜0.5 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,5S)-/(3S,5R)-1-(benzyloxycarbonyl)-5-methoxypiperidine-3-carboxylic acid (323 g) as a white solid, which was directly used in the next step without further purification. LCMS (m/z): 294.0 [M+H]+; Rt=0.71 min.

### Step 3-b: Preparation of 1-(benzyloxycarbonyl)-5-methylpiperidine-3-carboxylic acid [cis/trans isomers]

To a mixture of cis/trans isomers of 5-methoxy-piperidine-1,3-dicarboxylic acid 1-benzyl ester 3-methyl ester (596 mg, 1.94 mmol) in MeOH (1.95 mL) and water (1.3 mL) was added 6N aqueous sodium hydroxide solution (0.65 mL, 3.9 mmol). The reaction mixture was stirred at 25° C. for 2 hrs and concentrated under reduced pressure to a volume of ˜0.5 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of cis/trans isomers of 1-(benzyloxycarbonyl)-5-methoxypiperidine-3-carboxylic acid (530 mg) as a colorless oil, which was directly used in the next step without further purification. LCMS (m/z): 294.0 [M+H]+; Rt=0.71 min.

### SYNTHETIC EXAMPLES

### Example 3

### (R)-1-(2-Methoxy-ethyl)-piperidine-3-carboxylic acid (5-chloro-4-{6-[tetrahydro-pyran-4-ylmethyl)-amino]-pyrazin-2-yl}-pyridin-2-yl)-amide

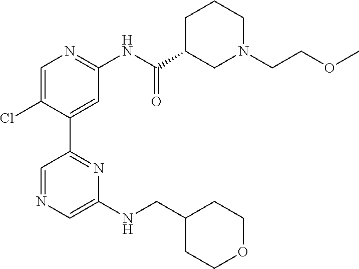

A mixture of (R)—N-(5-chloro-4-(6-(((tetrahydro-2H-pyran-4-yl)methyl)amino)pyrazin-2-yl)pyridin-2-yl)piperidine-3-carboxamide (15 mg, 0.035 mmol, DMSO (0.5 mL), potassium carbonate (7.70 mg, 0.056 mmol) and 1-bromo-2-methoxyethane (6.77 mg, 0.049 mmol) was stirred at 50° C. for 2 hrs. The reaction mixture was cooled to room temperature, diluted with DMSO (0.5 mL), filtered through a syringe filter and purified by HPLC. Fractions were collected and lyophilized providing (R)-1-(2-methoxy-ethyl)-piperidine-3-carboxylic acid (5-chloro-4-{6-[(tetrahydro-pyran-4-ylmethyl)-amino]-pyrazin-2-yl}-pyridin-2-yl)-amide (10.4 mg) as its trifluoroacetic acid salt as an off-white solid. LCMS (m/z): 489.2 [M+H]+; Rt=0.57 min.

### Example 6

### (S)-1-Methanesulfonyl-piperidine-3-carboxylic acid (5-chloro-4-{6-[tetrahydro-pyran-4-ylmethyl)-amino]-pyrazin-2-yl}-pyridin-2-yl)-amide

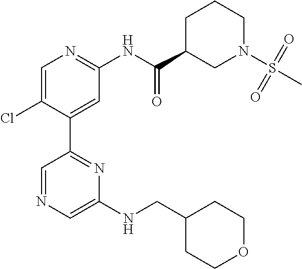

A mixture of (S)—N-(5-chloro-4-(6-(((tetrahydro-2H-pyran-4-yl)methyl)amino)-pyrazin-2-yl)pyridin-2-yl)piperidine-3-carboxamide (11 mg, 0.026 mmol), DCM (0.8 mL), triethylamine (5.34 μL, 0.038 mmol) and methanesulfonyl chloride (2.4 μL, 0.031 mmol) was stirred at room temperature for 1 hr. The volatile solvent was removed under reduced pressure. The residue was dissolved in of DMSO (0.9 mL), filtered through a syringe filter and purified by HPLC. Fractions were collected and lyophilized providing (S)-1-methanesulfonyl-piperidine-3-carboxylic acid (5-chloro-4-{6-[(tetrahydro-pyran-4-ylmethyl)-amino]-pyrazin-2-yl}-pyridin-2-yl)-amide (7.4 mg) as its trifluoroacetic acid salt as an off-white solid. LCMS (m/z): 509.1 [M+H]+; Rt=0.69 min.

### Example 8

### (S)-Piperidine-3-carboxylic acid (5-chloro-4-{6-[(tetrahydro-pyran-4-ylmethyl)-amino]-pyrazin-2-yl}-pyridin-2-yl)-amide

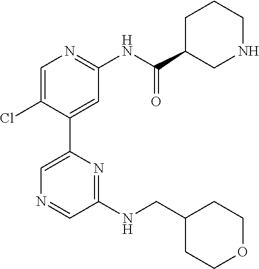

### Step 1: Preparation of (S)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate

To a solution of (S)-1-(tert-butoxycarbonyl)piperidine-3-carboxylic acid (252 mg, 1.1 mmol) in DCM (2.5 mL) was added 1-chloro-N,N,2-trimethylprop-1-en-1-amine (147 mg, 1.1 mmol). The mixture was stirred at room temperature for 10 min and was added to a solution of 6-(2-amino-5-chloropyridin-4-yl)-N-((tetrahydro-2H-pyran-4-yl)methyl)pyrazin-2-amine (320 mg, 1.0 mmol) and pyridine (87 mg, 1.1 mmol) in THF (5 mL). The reaction mixture was stirred at 23° C. for 2 hrs. The reaction mixture was concentrated under reduced pressure and diluted with water (10 mL). The mixture was extracted with EtOAc (2×15 mL). The combined organic layers were concentrated under reduced pressure and the residue was purified by column chromatography [silica gel, EtOAc/heptane=0/100 to 30/70]. Fractions were collected and concentrated under reduced pressure providing (S)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate. LCMS (m/z): 531.1 [M+H]+; Rt=0.97 min.

### Step 2: Preparation of (S)-piperidine-3-carboxylic acid (5-chloro-4-{6-[(tetrahydro-pyran-4-ylmethyl)-amino]-pyrazin-2-yl}-pyridin-2-yl)-amide

A mixture of (S)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate and 4M hydrochloride solution in dioxane (2 mL) was stirred at 23° C. for 2 hrs. The reaction mixture was diluted with saturated aqueous sodium bicarbonate solution (4 mL) and the volatile solvent was removed under reduced pressure. The residue was extracted with EtOAc (3×20 mL), the combined organic layers were concentrated under reduced pressure providing (S)-piperidine-3-carboxylic acid (5-chloro-4-{6-[(tetrahydro-pyran-4-ylmethyl)-amino]-pyrazin-2-yl}-pyridin-2-yl)-amide (300 mg). LCMS (m/z): 431.1 [M+H]+; Rt=0.71 min.

### Example 9

### (R)-Piperidine-3-carboxylic acid (5-chloro-4-{6-[(tetrahydro-pyran-4-ylmethyl)-amino]-pyrazin-2-yl}-pyridin-2-yl)-amide

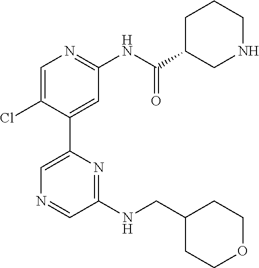

### Step 1: Preparation of (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate

A mixture of (R)-1-(tert-butoxycarbonyl)piperidine-3-carboxylic acid (60.2 mg, 0.263 mmol) and HATU (143 mg, 0.375 mmol) in acetonitrile (1.5 mL) and NMP (0.5 mL) was stirred for ˜30 min. 6-(2-Amino-5-chloropyridin-4-yl)-N-((tetrahydro-2H-pyran-4-yl)methyl)pyrazin-2-amine (40 mg, 0.125 mmol), dissolved in NMP (0.5 mL), and DIPEA (0.100 mL, 0.575 mmol) were added and the mixture was heated in a sealed tube at 70° C. for ˜16 hrs. Additional (R)-1-(tert-butoxycarbonyl)piperidine-3-carboxylic acid (60.2 mg, 0.263 mmol), HATU (143 mg, 0.375 mmol) in acetonitrile (0.6 mL) and NMP (0.200 mL), which was stirred for ˜1 hr, and DIPEA (0.100 mL, 0.575 mmol) were added and heating was continued for ˜25 hrs. The mixture was diluted with EtOAc (˜40 mL). The organic phase was washed with saturated aqueous sodium bicarbonate solution, brine and concentrated under reduced pressure. The residue was dissolved in DMSO (˜2.5 mL), filtered through a syringe filter and purified by HPLC. Fractions were collected and lyophilized providing (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylates (14.3 mg) as its trifluoroacetic acid salt. LCMS (m/z): 531.1/533.2 [M+H]+; Rt=0.98 min.

### Step 2: Preparation of (R)-piperidine-3-carboxylic acid (5-chloro-4-{6-[(tetrahydro-pyran-4-ylmethyl)-amino]-pyrazin-2-yl}-pyridin-2-yl)-amide

To a solution of (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate (12 mg) in MeOH (2 mL) was added 4M hydrochloride solution in dioxane (6 mL). The mixture was stirred for ˜30 min at room temperature. The mixture was concentrated under reduced pressure, dissolved in DMSO (1.3 mL), filtered through a syringe filter and purified by HPLC. Fractions were collected and lyophilized providing (R)-piperidine-3-carboxylic acid (5-chloro-4-{6-[(tetrahydro-pyran-4-ylmethyl)-amino]-pyrazin-2-yl}-pyridin-2-yl)-amide as its trifluoroacetic acid salt (11.6 mg). LCMS (m/z): 431.2/433.1 [M+H]+; Rt=0.59 min.

### Alternative preparation of (R)-piperidine-3-carboxylic acid (5-chloro-4-{6-[(tetrahydro-pyran-4-ylmethyl)-amino]-pyrazin-2-yl}-pyridin-2-yl)-amide

### Step 1: Preparation of (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate

To a solution of (R)-1-(tert-butoxycarbonyl)piperidine-3-carboxylic acid (495 mg, 2.158 mmol) in DCM (3 mL) at 0° C. was added 1-chloro-N,N,2-trimethylprop-1-en-1-amine (336 mg, 2.52 mmol). The mixture was stirred at room temperature for 30 min and added to a solution of 6-(2-amino-5-chloropyridin-4-yl)-N-((tetrahydro-2H-pyran-4-yl)methyl)pyrazin-2-amine (575 mg, 1.798 mmol) and pyridine (0.204 mL, 2.52 mmol) in THF (3 mL). The reaction mixture was stirred at room temperature for 2 hrs. The mixture was diluted with EtOAc (300 mL) and washed with saturated aqueous sodium bicarbonate solution (1×) and water (2×), filtered off and concentrated under reduced pressure. The residue was purified by column chromatography [silica gel, 24 g, EtOAc/heptane]. Fractions were combined and concentrated under reduced pressure providing (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate (14.3 mg). LCMS (m/z): 531.3 [M+H]+; Rt=1.01 min.

### Step 2: Preparation of (R)-piperidine-3-carboxylic acid (5-chloro-4-{6-[(tetrahydro-pyran-4-ylmethyl)-amino]-pyrazin-2-yl}-pyridin-2-yl)-amide

To a solution of (R)-tert-butyl 3-(5-chloro-4-(6-(((tetrahydro-2H-pyran-4-yl)methyl)amino)pyrazin-2-yl)pyridin-2-ylcarbamoyl)piperidine-1-carboxylate (1.798 mmol) was added 4M hydrochloride solution in dioxane (18 mL, 72.0 mmol). The mixture was stirred for 1 hr at room temperature. The mixture was concentrated under reduced pressure, dissolved in EtOAc (500 mL), washed with saturated aqueous sodium bicarbonate solution (1×), water (2×) and brine (1×), dried over sodium sulfate, filtered off and concentrated under reduced pressure. The residue was purified by column chromatography [silica gel, 24 g, DCM {1% triethylamine}/MeOH=100/0 to 92/8]. Fractions were combined and concentrated under reduced pressure, the residue was dissolved in acetonitrile/water (1/1), filtered through a syringe filter and lyophilized providing (R)-piperidine-3-carboxylic acid (5-chloro-4-{6-[(tetrahydro-pyran-4-ylmethyl)-amino]-pyrazin-2-yl}-pyridin-2-yl)-amide (510 mg). LCMS (m/z): 431.2 [M+H]+; Rt=0.57 min.

### Example 10

### N-(5-Chloro-4-{6-[(tetrahydro-pyran-4-ylmethyl)-amino]-pyrazin-2-yl}-pyridin-2-yl)-2-methoxy-acetamide

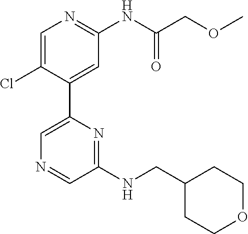

To a mixture of 6-(2-amino-5-chloropyridin-4-yl)-N-((tetrahydro-2H-pyran-4-yl)methyl)pyrazin-2-amine (21 mg, 0.066 mmol) and DIPEA (9.3 mg, 0.072 mmol) in DCM (0.3 mL) was added slowly methoxy acetylchloride (7.48 mg, 0.069 mmol). After stirring for 2 hrs at room temperature the mixture was diluted with water (0.5 mL) and stirred vigorously for 2 min. The volatile solvent was removed under reduced pressure and the mixture was diluted with DMF (2 mL). Purification by HPLC provided N-(5-chloro-4-{6-[(tetrahydro-pyran-4-ylmethyl)-amino]-pyrazin-2-yl}-pyridin-2-yl)-2-methoxy-acetamide as its trifluoroacetic acid salt (3 mg). LCMS (m/z): 392.1 [M+H]+; Rt=0.71 min.

### Example 11

### N-(5-Chloro-4-{6-[(tetrahydro-pyran-4-ylmethyl)-amino]-pyrazin-2-yl}-pyridin-2-yl)-acetamide

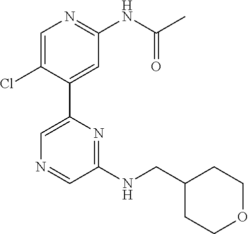

To a mixture of 6-(2-amino-5-chloropyridin-4-yl)-N-((tetrahydro-2H-pyran-4-yl)methyl)pyrazin-2-amine (21 mg, 0.066 mmol) and pyridine (6.2 mg, 0.079 mmol) in DCM (0.3 mL) was added acetyl chloride (5.7 mg, 0.072 mmol). After stirring for 2 hrs at room temperature the mixture was diluted with water (0.5 mL). The volatile solvent was removed under reduced pressure and the mixture was diluted with DMF (1.5 mL). Purification by HPLC provided N-(5-chloro-4-{6-[(tetrahydro-pyran-4-ylmethyl)-amino]-pyrazin-2-yl}-pyridin-2-yl)-acetamide as its trifluoroacetic acid salt (12 mg). LCMS (m/z): 362.2 [M+H]+; Rt=0.63 min.

### Example 14

### (R)-Piperidine-3-carboxylic acid {5-chloro-4-[6-(2-fluoro-benzylamino)-pyrazin-2-yl]-pyridin-2-yl}-amide

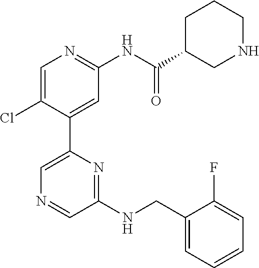

### Step 1: Preparation of (R)-tert-butyl 3-(5-chloro-4-(6-(2-fluorobenzylamino)pyrazin-2-yl)pyridin-2-ylcarbamoyl)piperidine-1-carboxylate

A mixture of (R)-tert-butyl 3-(5-chloro-4-(6-chloropyrazin-2-yl)pyridin-2-ylcarbamoyl)piperidine-1-carboxylate (20 mg, 0.044 mmol) and (2-fluorophenyl)methanamine (22 mg, 0.177 mmol) in DMSO (0.15 mL) was heated at 90° C. for 18 hrs. The reaction mixture was cooled to room temperature and was diluted with water (0.05 mL). Purification by HPLC provided (R)-tert-butyl 3-(5-chloro-4-(6-(2-fluorobenzylamino)pyrazin-2-yl)pyridin-2-ylcarbamoyl)piperidine-1-carboxylate as its trifluoroacetic acid salt. LCMS (m/z): 541.3 [M+H]+; Rt=1.12 min.

### Step 2: Preparation of (R)-Piperidine-3-carboxylic acid {5-chloro-4-[6-(2-fluoro-benzylamino)-pyrazin-2-yl]-pyridin-2-yl}-amide

(R)-tert-butyl 3-(5-chloro-4-(6-(2-fluorobenzylamino)pyrazin-2-yl)pyridin-2-ylcarbamoyl)piperidine-1-carboxylate (trifluoroacetic acid salt) was dissolved in a mixture of DCM (0.5 mL) and trifluoroacetic acid (0.5 mL). The mixture was stirred at room temperature for 1 hr and concentrated under reduced pressure. The residue was dissolved in DMSO (0.2 mL) and purified by HPLC providing (R)-piperidine-3-carboxylic acid {5-chloro-4-[6-(2-fluoro-benzylamino)-pyrazin-2-yl]-pyridin-2-yl}-amide as its trifluoroacetic acid salt (10 mg). LCMS (m/z): 441.1 [M+H]+; Rt=0.64 min.

### Example 24

### (R)-Piperidine-3-carboxylic acid (5-chloro-4-{3-methyl-6-[(tetrahydro-pyran-4-ylmethyl)-amino]-pyrazin-2-yl}-pyridin-2-yl)-amide

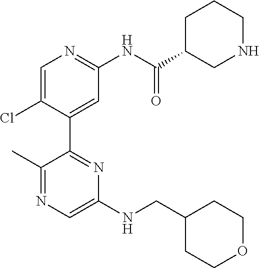

### Step 1: Preparation of (3R)-tert-butyl 3-(5-chloro-4-(3-methyl-6-(((tetrahydro-2H-pyran-4-yl)methyl)amino)pyrazin-2-yl)pyridin-2-ylcarbamoyl)piperidine-1-carboxylate

To a solution of (R)-1-(tert-butoxycarbonyl)piperidine-3-carboxylic acid (0.030 g, 0.133 mmol) in DCM (0.50 mL) under argon was added 1-chloro-N,N,2-trimethylprop-1-en-1-amine (0.020 mL, 0.021 g, 0.155 mmol) at 0° C. The mixture was stirred at room temperature for 30 min and added to a solution of 6-(2-amino-5-chloropyridin-4-yl)-5-methyl-N-((tetrahydro-2H-pyran-4-yl)methyl)pyrazin-2-amine (0.0369 g, 0.111 mmol) and pyridine (0.013 mL, 0.012 g, 0.155 mmol) in THF (0.50 mL). The reaction mixture was stirred at room temperature for 30 min and diluted with saturated aqueous sodium bicarbonate solution (25 mL). The mixture was extracted with EtOAc (3×25 mL) and the combined organic layers were washed with brine (lx 25 mL), dried over sodium sulfate, filtered off and concentrated under reduced pressure. The residue was purified by column chromatography [silica gel, EtOAc/heptane=25/75 to 100/0]. Fractions were combined and concentrated under reduced pressure providing (3R)-tert-butyl 3-(5-chloro-4-(3-methyl-6-(((tetrahydro-2H-pyran-4-yl)methyl)amino)-pyrazin-2-yl)pyridin-2-ylcarbamoyl)piperidine-1-carboxylate (0.0331 g). LCMS (m/z): 545.3 [M+H]+; Rt=0.92 min.

### Step 2: Preparation of (R)-Piperidine-3-carboxylic acid (5-chloro-4-{3-methyl-6-[(tetrahydro-pyran-4-ylmethyl)-amino]-pyrazin-2-yl}-pyridin-2-yl)-amide

To a solution of (3R)-tert-butyl 3-(5-chloro-4-(3-methyl-6-(((tetrahydro-2H-pyran-4-yl)methyl)amino)pyrazin-2-yl)pyridin-2-ylcarbamoyl)piperidine-1-carboxylate (33.1 mg, 0.061 mmol) in MeOH (0.2 mL) was added 4M hydrochloride solution in dioxane (1.5 mL). The reaction mixture was stirred at room temperature for 1 hr and concentrated under reduced pressure. The residue was purified by HPLC providing (R)-piperidine-3-carboxylic acid (5-chloro-4-{3-methyl-6-[(tetrahydro-pyran-4-ylmethyl)-amino]-pyrazin-2-yl}-pyridin-2-yl)-amide as its trifluoroacetic acid salt. (9.2 mg). LCMS (m/z): 445.2 [M+H]+; Rt=0.56 min.

### Example 45

### (S)-1-Ethanesulfonyl-piperidine-3-carboxylic acid (5-chloro-4-{6-[(tetrahydro-pyran-4-ylmethyl)-amino]-pyrazin-2-yl}-pyridin-2-yl)-amide

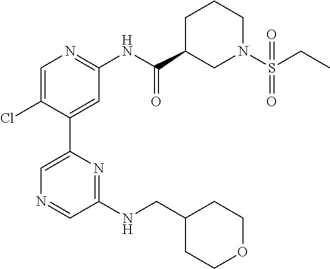

To a solution of (S)—N-(5-chloro-4-(6-(((tetrahydro-2H-pyran-4-yl)methyl)amino)pyrazin-2-yl)pyridin-2-yl)piperidine-3-carboxamide (20 mg, 0.05 mmol) in DCM (0.3 mL) was added triethylamine (10 mg). The mixture was stirred for ˜2 min at 23° C. Ethanesulfonyl chloride (6 mg, 0.05 mmol) was added and the reaction mixture was stirred in a sealed vial at 23° C. for 45 min. The reaction mixture was concentrated under reduced pressure and the residue was dissolved in DMF (2 mL) and filtered through a syringe filter. Purification by HPLC provided (S)-1-ethanesulfonyl-piperidine-3-carboxylic acid (5-chloro-4-{6-[(tetrahydro-pyran-4-ylmethyl)-amino]-pyrazin-2-yl}-pyridin-2-yl)-amide as its trifluoroacetic acid salt. LCMS (m/z): 523.2 [M+H]+; Rt=0.80 min.

### Example 46

### (S)-3-(5-Chloro-4-{6-[(tetrahydro-pyran-4-ylmethyl)-amino]-pyrazin-2-yl}-pyridin-2-ylcarbamoyl)-piperidine-1-carboxylic acid isopropyl ester

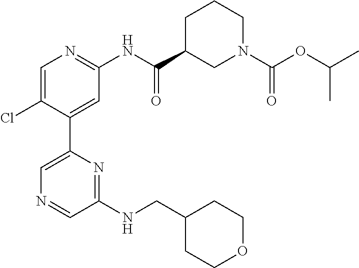

To a solution of (S)—N-(5-chloro-4-(6-(((tetrahydro-2H-pyran-4-yl)methyl)amino)pyrazin-2-yl)pyridin-2-yl)piperidine-3-carboxamide (20 mg, 0.05 mmol) in DCM (0.3 mL) was added triethylamine (10 mg). The mixture was stirred for ˜2 min at 23° C. Isopropyl carbonochloridate (6.2 mg, 0.05 mmol) was added and the reaction mixture was stirred in a sealed vial at 23° C. for 45 min. The mixture was diluted with water (0.1 mL) and vigorously stirred for 5 min. The mixture was concentrated under reduced pressure and the resulting residue was dissolved in DMF (2 mL) and filtered through a syringe filter. Purification by HPLC provided (S)-3-(5-chloro-4-{6-[(tetrahydro-pyran-4-ylmethyl)-amino]-pyrazin-2-yl}-pyridin-2-ylcarbamoyl)-piperidine-1-carboxylic acid isopropyl ester as its trifluoroacetic acid salt. LCMS (m/z): 517.2 [M+H]+; Rt=0.91 min.

### Example 47

### (S)-1-(2-Methoxy-acetyl)-piperidine-3-carboxylic acid (5-chloro-4-{6-[(tetrahydro-pyran-4-ylmethyl)-amino]-pyrazin-2-yl}-pyridin-2-yl)-amide

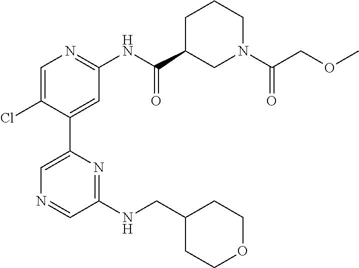

To a solution of (S)—N-(5-chloro-4-(6-(((tetrahydro-2H-pyran-4-yl)methyl)amino)pyrazin-2-yl)pyridin-2-yl)piperidine-3-carboxamide (20 mg, 0.05 mmol) in DCM (0.3 mL) was added triethylamine (10 mg). The mixture was stirred for ˜2 min at 23° C. 2-Methoxyacetyl chloride (5.5 mg, 0.05 mmol) was added and the reaction mixture was stirred in a sealed vial at 23° C. for 45 min. The mixture was diluted with water (0.1 mL) and vigorously stirred for 5 min. The mixture was concentrated under reduced pressure and the resulting residue was dissolved in DMF (2 mL) and filtered through a syringe filter. Purification by HPLC provided (S)-1-(2-methoxy-acetyl)-piperidine-3-carboxylic acid (5-chloro-4-{6-[(tetrahydro-pyran-4-ylmethyl)-amino]-pyrazin-2-yl}-pyridin-2-yl)-amide as its trifluoroacetic acid salt. LCMS (m/z): 503.2 [M+H]+; Rt=0.69 min.

### Example 50

### (1S,3R)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide

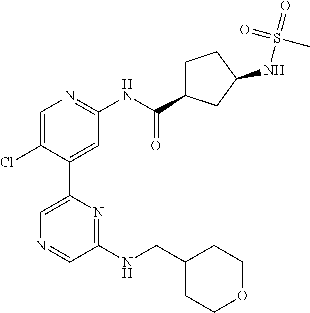

### Step 1: Preparation of (1S,3R)-3-amino-N-(5-chloro-4-(6-(((tetrahydro-2H-pyran-4-yl)methyl)amino)pyrazin-2-yl)pyridin-2-yl)cyclopentanecarboxamide

To a solution of (1S,3R)-3-(tert-butoxycarbonylamino)cyclopentanecarboxylic acid (64.5 mg, 0.28 mmol) in DCM (0.6 mL) was added 1-chloro-N,N,2-trimethylprop-1-en-1-amine (30 mg, 0.225 mmol). The mixture was stirred at room temperature for 30 min and was added to a solution of 6-(2-amino-5-chloropyridin-4-yl)-N-((tetrahydro-2H-pyran-4-yl)methyl)pyrazin-2-amine (60 mg, 0.188 mmol) and pyridine (16.3 mg, 0.21 mmol) in THF (1.2 mL). The reaction mixture was stirred at room temperature for 2 hrs. The mixture was concentrated under reduced pressure providing crude tert-butyl (1R,3S)-3-(5-chloro-4-(6-(((tetrahydro-2H-pyran-4-yl)methyl)amino)pyrazin-2-yl)pyridin-2-ylcarbamoyl)cyclopentylcarbamate, which was directly used in the next step without further purification. LCMS (m/z): 509 [M+H]+; Rt=0.71 min.

### Step 2: Preparation of (1S,3R)-3-amino-N-(5-chloro-4-(6-(((tetrahydro-2H-pyran-4-yl)methyl)amino)pyrazin-2-yl)pyridin-2-yl)cyclopentanecarboxamide

To crude tert-butyl (1R,3S)-3-(5-chloro-4-(6-(((tetrahydro-2H-pyran-4-yl)methyl)amino)pyrazin-2-yl)pyridin-2-ylcarbamoyl)cyclopentylcarbamate was added 4M hydrochloride solution in dioxane (2 mL, 8.00 mmol) and the mixture was stirred for 90 min at room temperature. The mixture was carefully diluted with saturated aqueous sodium bicarbonate solution (3 mL). The mixture was concentrated under reduced pressure, diluted with DMF (2 mL), sonicated (5 mL) and filtered through a syringe filter. Purification by HPLC provided (1S,3R)-3-amino-N-(5-chloro-4-(6-(((tetrahydro-2H-pyran-4-yl)methyl)amino)pyrazin-2-yl)pyridin-2-yl)cyclopentanecarboxamide as its trifluoroacetic acid salt (45 mg). LCMS (m/z): 431.1 [M+H]+; Rt=0.58 min.

### Step 3: Preparation of (1S,3R)-3-methanesulfonylamino-cyclopentanecarboxylic acid (5-chloro-4-{6-[(tetrahydro-pyran-4-ylmethyl)-amino]-pyrazin-2-yl}-pyridin-2-yl)-amide

To a mixture of (1S,3R)-3-amino-N-(5-chloro-4-(6-(((tetrahydro-2H-pyran-4-yl)methyl)amino)pyrazin-2-yl)pyridin-2-yl)cyclopentanecarboxamide (I50B, 24 mg, 0.056 mmol) and triethylamine (11.3 mg, 0.111 mmol) in DCM (0.6 mL) was added methanesulfonyl chloride (6.38 mg, 0.056 mmol). After stirring for 90 min at room temperature the mixture was diluted with water (0.6 mL). The mixture was concentrated under reduced pressure and diluted with DMF (1.4 mL). Purification by HPLC provided (1S,3R)-3-methanesulfonylamino-cyclopentanecarboxylic acid (5-chloro-4-{6-[(tetrahydro-pyran-4-ylmethyl)-amino]-pyrazin-2-yl}-pyridin-2-yl)-amide (12.8 mg) as its trifluoroacetic acid salt. LCMS (m/z): 509 [M+H]+; Rt=0.71 min.

### Example 57

### N-(5-Chloro-4-{6-[(tetrahydro-pyran-4-ylmethyl)-amino]-pyrazin-2-yl}-pyridin-2-yl)-2-pyridin-3-yl-acetamide

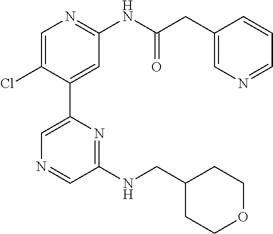

To a solution of 2-(pyridin-3-yl)acetic acid (8.6 mg, 0.06 mmol) in DCM (0.2 mL) was added 1-chloro-N,N,2-trimethylprop-1-en-1-amine (8.36 mg, 0.063 mmol) and the mixture was stirred at 23° C. for 30 min. To this mixture was added a solution of 6-(2-amino-5-chloropyridin-4-yl)-N-((tetrahydro-2H-pyran-4-yl)methyl)pyrazin-2-amine (20 mg, 0.06 mmol) and triethylamine (0.012 mL) in THF (0.4 mL) and the resulting reaction mixture was stirred at 23° C. for 2 hrs. The reaction mixture was diluted with water (0.2 mL) and stirred vigorously for 2 min. The mixture was concentrated under reduced pressure and the residue was dissolved in DMF (2 mL) and sonicated for 5 min. The mixture was filtered through a syringe filter and purified by HPLC. Fractions were collected and lyophilized providing N-(5-chloro-4-{6-[(tetrahydro-pyran-4-ylmethyl)-amino]-pyrazin-2-yl}-pyridin-2-yl)-2-pyridin-3-yl-acetamide as its trifluoroacetic acid salt. LCMS (m/z): 439.1 [M+H]+; Rt=0.57 min.

### Example 59

Tetrahydro-pyran-4-carboxylic acid (4-{6-[(tetrahydro-pyran-4-ylmethyl)-amino]-pyrazin-2-yl}-5-trifluoromethyl-pyridin-2-yl)-amide

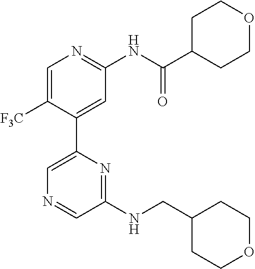

### Step 1: Preparation of 4-iodo-5-(trifluoromethyl)pyridin-2-amine

A mixture of 2-chloro-4-iodo-5-(trifluoromethyl)pyridine (600 mg, 1.952 mmol) and ammonium acetate (2.29 g, 29.7 mmol) in NMP (2 mL) was heated at 110° C. for 17 hrs and then cooled to room temperature. The mixture was partitioned between EtOAc and water. The aqueous layer was extracted with EtOAc and the combined organic layers were washed with water (3×) and brine (3×), dried over sodium sulfate, filtered off and concentrated under reduced pressure. The residue was purified by column chromatography [silica gel, EtOAc/heptane=25/75 to 50/50] providing 4-iodo-5-(trifluoromethyl)pyridin-2-amine (215 mg).

### Step 2: Preparation of 4-(6-chloropyrazin-2-yl)-5-(trifluoromethyl)pyridin-2-amine

To a solution of 4-iodo-5-(trifluoromethyl)pyridin-2-amine (181 mg, 0.628 mmol) and 2-chloro-6-(4,4,5,5-tetramethyl-1,3,2-dioxaborolan-2-yl)pyrazine (453 mg, 1.884 mmol) in DME (4 mL) and 2M aqueous sodium carbonate solution (2 mL) was added PdCl₂(dppf) CH₂Cl₂ adduct (51.3 mg, 0.063 mmol). The mixture was heated at 90° C. for 2 hrs and then cooled to room temperature and diluted with EtOAc. The separated organic layer was washed with 1M aqueous sodium hydroxide solution (1×), water (1×) and brine (2×), dried over sodium sulfate, filtered off and concentrated under reduced pressure. The residue was purified by column chromatography [silica gel, EtOAc/heptane=25/75 to 50/50] providing 4-(6-chloropyrazin-2-yl)-5-(trifluoromethyl)pyridin-2-amine (98 mg). LCMS (m/z): 275.9 [M+H]+; Rt=0.59 min.

### Step 3: Preparation of 6-(2-amino-5-(trifluoromethyl)pyridin-4-yl)-N-((tetrahydro-2H-pyran-4-yl)methyl)pyrazin-2-amine

A mixture of 4-(6-chloropyrazin-2-yl)-5-(trifluoromethyl)pyridin-2-amine (98 mg, 0.357 mmol), (tetrahydro-2H-pyran-4-yl)methanamine (45.2 mg, 0.393 mmol), DIPEA (0.093 mL, 0.535 mmol), and DMSO (0.2 mL) was heated at 110° C. for 22 hrs. The reaction mixture was cooled to room temperature and was diluted with EtOAc. The separated organic layer was washed with water (1×) and brine (2×), dried over sodium sulfate, filtered off and concentrated under reduced pressure. The residue was used in the next step without further purification. LCMS (m/z): 354.9 [M+H]+; Rt=0.53 min.

### Step 4: Preparation of tetrahydro-pyran-4-carboxylic acid (4-{6-[(tetrahydro-pyran-4-ylmethyl)-amino]-pyrazin-2-yl}-5-trifluoromethyl-pyridin-2-yl)-amide

To a mixture of 6-(2-amino-5-(trifluoromethyl)pyridin-4-yl)-N-((tetrahydro-2H-pyran-4-yl)methyl)pyrazin-2-amine (14 mg, 0.040 mmol) and pyridine (4.17 μL, 0.052 mmol) in DCM (0.5 mL) was added tetrahydro-2H-pyran-4-carbonyl chloride (6.48 mg, 0.044 mmol). The mixture was stirred for 1 hr and then concentrated under reduced pressure. The residue was purified by HPLC. Fractions were combined and lyophilized providing tetrahydro-pyran-4-carboxylic acid (4-{6-[(tetrahydro-pyran-4-ylmethyl)-amino]-pyrazin-2-yl}-5-trifluoromethyl-pyridin-2-yl)-amide (5.9 mg) as its trifluoroacetic acid salt. LCMS (m/z): 466.1 [M+H]+; Rt=0.76 min.

### Example 60

### N-(5-Methyl-4-{6-[(tetrahydro-pyran-4-ylmethyl)-amino]-pyrazin-2-yl}-pyridin-2-yl)-isobutyramide

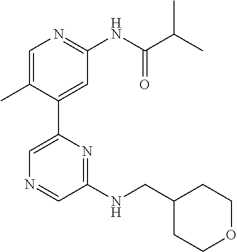

### Step 1: Preparation of 4-iodo-5-methylpyridin-2-amine

A mixture of 2-fluoro-4-iodo-5-methylpyridine (758 mg, 3.20 mmol) and ammonium acetate (5.0 g, 64.9 mmol) in NMP (3 mL) was heated at 110° C. for 6 days and then cooled to room temperature. The reaction mixture was diluted with EtOAc and water. The separated organic layer was washed with water (3×) and brine (2×), dried over sodium sulfate, filtered off and concentrated under reduced pressure. The residue was purified by column chromatography [silica gel, EtOAc/heptane=25/75 to 50/50] providing 4-iodo-5-methylpyridin-2-amine (60 mg).

### Step 2: Preparation of 4-(6-chloropyrazin-2-yl)-5-methylpyridin-2-amine

To a solution of 4-iodo-5-methylpyridin-2-amine (60 mg, 0.256 mmol), 2-chloro-6-(4,4,5,5-tetramethyl-1,3,2-dioxaborolan-2-yl)pyrazine (247 mg, 1.025 mmol) in DME (1 mL) and 2M aqueous sodium carbonate solution (0.5 mL) was added PdCl₂(dppf) CH₂Cl₂ adduct (20.94 mg, 0.026 mmol). The mixture was purged with argon, heated at 100° C. for 5 hrs, and then cooled to room temperature and diluted with EtOAc. The mixture was washed with 1M aqueous sodium hydroxide solution (1×), water (1×) and brine (2×), dried over sodium sulfate, filtered off and concentrated under reduced pressure providing crude 4-(6-chloropyrazin-2-yl)-5-methylpyridin-2-amine (64 mg), which was used directly in next step without further purification.

### Step 3: Preparation of 6-(2-amino-5-methylpyridin-4-yl)-N-((tetrahydro-2H-pyran-4-yl)methyl)pyrazin-2-amine

A mixture of crude 4-(6-chloropyrazin-2-yl)-5-methylpyridin-2-amine (55 mg, 0.25 mmol), (tetrahydro-2H-pyran-4-yl)methanamine (57.6 mg, 0.500 mmol), DIPEA (0.087 mL, 0.500 mmol), and DMSO (0.2 mL) was heated at 110° C. for 18 hrs. The reaction mixture was cooled to room temperature and was diluted with EtOAc and water. The separated organic layer was washed with water (3×) and brine (2×), dried over sodium sulfate, filtered off and concentrated under reduced pressure. The residue was purified by column chromatography [silica gel, MeOH/DCM=2/98 to 10/90] providing 6-(2-amino-5-methylpyridin-4-yl)-N-((tetrahydro-2H-pyran-4-yl)methyl)pyrazin-2-amine (31 mg). LCMS (m/z): 300.9 [M+H]+; Rt=0.45 min.

### Step 4: Preparation of N-(5-methyl-4-{6-[(tetrahydro-pyran-4-ylmethyl)-amino]-pyrazin-2-yl}-pyridin-2-yl)-isobutyramide

To a mixture of 6-(2-amino-5-methylpyridin-4-yl)-N-((tetrahydro-2H-pyran-4-yl)methyl)pyrazin-2-amine (8.0 mg, 0.027 mmol) and pyridine (6.48 μL, 0.080 mmol) in DCM (0.2 mL) was added isobutyryl chloride (3.10 μL, 0.029 mmol). The mixture was stirred for 4 hrs and then concentrated under reduced pressure. The residue was purified by HPLC. Fractions were combined and lyophilized providing N-(5-methyl-4-{6-[(tetrahydro-pyran-4-ylmethyl)-amino]-pyrazin-2-yl}-pyridin-2-yl)-isobutyramide (1.7 mg) as its trifluoroacetic acid salt. LCMS (m/z): 370.1 [M+H]+; Rt=0.59 min.

### Example 64

(3R,4S)-4-Fluoro-pyrrolidine-3-carboxylic acid (5-chloro-4-{6-[(tetrahydro-pyran-4-ylmethyl)-amino]-pyrazin-2-yl}-pyridin-2-yl)-amide

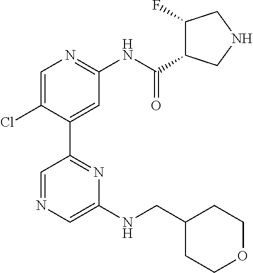

### Step 1: Preparation of (3R,4S)-3-(5-chloro-4-{6-[(tetrahydro-pyran-4-ylmethyl)-amino]-pyrazin-2-yl}-pyridin-2-ylcarbamoyl)-4-fluoro-pyrrolidine-1-carboxylic acid benzyl ester

A mixture of (3R,4S)-1-(benzyloxycarbonyl)-4-fluoropyrrolidine-3-carboxylic acid (109 mg, 0.407 mmol) and 1-chloro-N,N,2-trimethylprop-1-en-1-amine (71 mg, 0.532 mmol) in DCM (25 mL) was stirred in a sealed tube at 0° C. for 30 min. The mixture was added to a mixture of 6-(2-amino-5-chloropyridin-4-yl)-N-((tetrahydro-2H-pyran-4-yl)methyl)pyrazin-2-amine (100 mg, 0.313 mmol) and pyridine (49.5 mg, 0.625 mmol). The resulting reaction mixture was stirred at room temperature for 1 hr and was diluted with EtOAc (30 mL), washed with water (3×) and brine (1×), dried sodium sulfate, filtered off and concentrated under reduced pressure. The residue was purified by column chromatography [silica gel, 12 g, EtOAc/heptane=25/75 to 75/25]. Fractions were combined and concentrated under reduced pressure providing (3R,4S)-3-(5-chloro-4-{6-[(tetrahydro-pyran-4-ylmethyl)-amino]-pyrazin-2-yl}-pyridin-2-ylcarbamoyl)-4-fluoro-pyrrolidine-1-carboxylic acid benzyl ester (81 mg) as an off-white solid. LCMS (m/z): 569.2 [M+H]+; Rt=0.88 min.

### Step 2: Preparation of (3R,4S)-4-fluoro-pyrrolidine-3-carboxylic acid (5-chloro-4-{6-[(tetrahydro-pyran-4-ylmethyl)-amino]-pyrazin-2-yl}-pyridin-2-yl)-amide

A mixture of (3R,4S)-3-(5-chloro-4-{6-[(tetrahydro-pyran-4-ylmethyl)-amino]-pyrazin-2-yl}-pyridin-2-ylcarbamoyl)-4-fluoro-pyrrolidine-1-carboxylic acid benzyl ester (18 mg, 0.032 mmol) and Pd/C (10 wt. %, 2 mg) in methanol (5 mL) was stirred under hydrogen atmosphere (balloon) at room temperature for 3 hrs. The reaction mixture was filtered through a layer of celites and washed with EtOAc (5 mL). The filtrate was diluted with EtOAc (30 mL), washed with water (3×) and brine (1×), dried over sodium sulfate, filtered off and concentrated under reduced pressure. The residue was purified by HPLC. Fractions were combined and lyophilized providing (3R,4S)-4-fluoro-pyrrolidine-3-carboxylic acid (5-chloro-4-{6-[(tetrahydro-pyran-4-ylmethyl)-amino]-pyrazin-2-yl}-pyridin-2-yl)-amide (8 mg) as its trifluoroacetic acid salt as an off-white solid. LCMS (m/z): 435.1 [M+H]+; Rt=0.55 min.

Table 1 below provides a list of compounds that were prepared using the procedures outlined above, and by using the appropriate starting materials.

TABLE 1

| Ex. | | | Retention | |
|---|---|---|---|---|
| No. | Structure | M + H | Time [min] | Name |
| 1 | 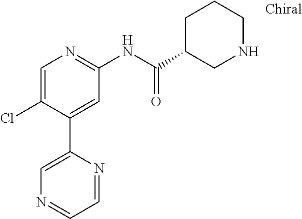 | 318.1 | 0.47 | (R)-Piperidine-3-carboxylic acid (5-chloro-4-pyrazin-2- yl-pyridin-2-yl)-amide |
| 2 | 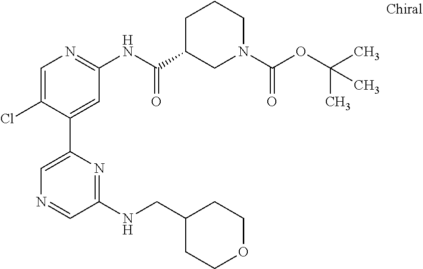 | 531.1 | 0.98 | (R)-3-(5-Chloro-4-{6- [(tetrahydro-pyran-4- ylmethyl)-amino]-pyrazin-2- yl}-pyridin-2-ylcarbamoyl)- piperidine-1-carboxylic acid tert-butyl ester |
| 3 | 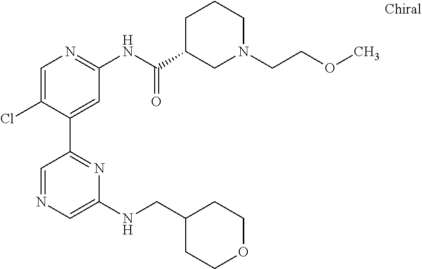 | 489.2 | 0.57 | (R)-1-(2-Methoxy-ethyl)- piperidine-3-carboxylic acid (5-chloro-4-{6-[(tetrahydro- pyran-4-ylmethyl)-amino]- pyrazin-2-yl}-pyridin-2-yl)- amide |
| 4 | 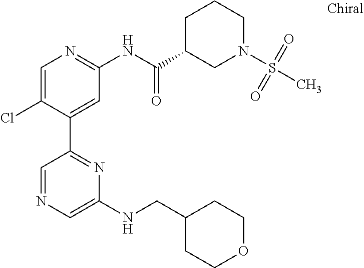 | 509.1 | 0.69 | (R)-1-Methanesulfonyl- piperidine-3-carboxylic acid (5-chloro-4-{6-[(tetrahydro- pyran-4-ylmethyl)-amino]- pyrazin-2-yl}-pyridin-2-yl)- amide |
| 5 | 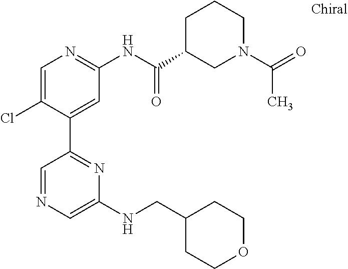 | 473.2 | 0.64 | (R)-1-Acetyl-piperidine-3- carboxylic acid (5-chloro-4- {6-[(tetrahydro-pyran-4- ylmethyl)-amino]-pyrazin-2- yl}-pyridin-2-yl)-amide |
| 6 | 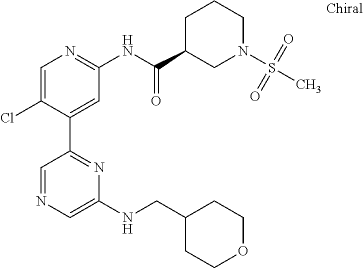 | 509.2 | 0.69 | (S)-1-Methanesulfonyl- piperidine-3-carboxylic acid (5-chloro-4-{6-[(tetrahydro- pyran-4-ylmethyl)-amino]- pyrazin-2-yl}-pyridin-2-yl)- amide |
| 7 | 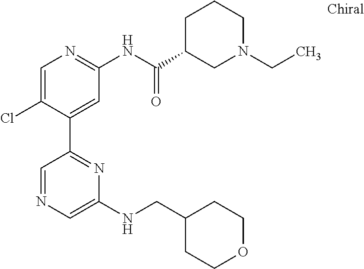 | 459.2 | 0.56 | (R)-1-Ethyl-piperidine-3- carboxylic acid (5-chloro-4- {6-[(tetrahydro-pyran-4- ylmethyl)-amino]-pyrazin-2- yl}-pyridin-2-yl)-amide |
| 8 | 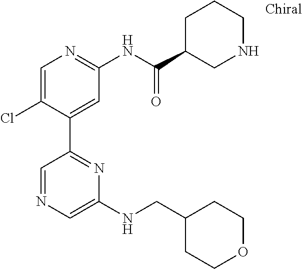 | 431.2 | 0.55 | (S)-Piperidine-3-carboxylic acid (5-chloro-4-{6- [(tetrahydro-pyran-4- ylmethyl)-amino]-pyrazin-2- yl}-pyridin-2-yl)-amide |
| 9 | 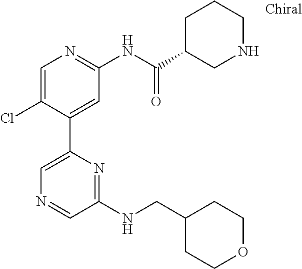 | 431.2 | 0.51 | (R)-Piperidine-3-carboxylic (5-chloro-4-{6- [(tetrahydro-pyran-4- ylmethyl)-amino]-pyrazin-2- yl}-pyridin-2-yl)-amide |
| 10 | 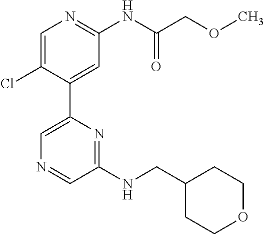 | 392.1 | 0.71 | N-(5-Chloro-4-{6- [(tetrahydro-pyran-4- ylmethyl)-amino]-pyrazin-2- yl}-pyridin-2-yl)-2-methoxy- acetamide |
| 11 | 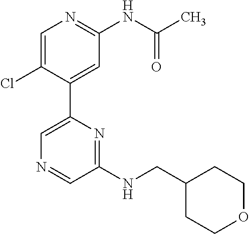 | 362.2 | 0.63 | N-(5-Chloro-4-{6- [(tetrahydro-pyran-4- ylmethyl)-amino]-pyrazin-2- yl}-pyridin-2-yl)-acetamide |
| 12 | 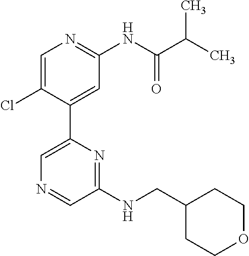 | 390.1 | 0.75 | N-(5-Chloro-4-{6- [(tetrahydro-pyran-4- ylmethyl)-amino]-pyrazin-2- yl}-pyridin-2-yl)- isobutyramide |
| 13 | 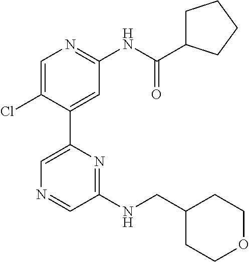 | 416.1 | 0.84 | Cyclopentanecarboxylic acid (5-chloro-4-{6-[(tetrahydro- pyran-4-ylmethyl)-amino]- pyrazin-2-yl}-pyridin-2-yl)- amide |
| 14 | 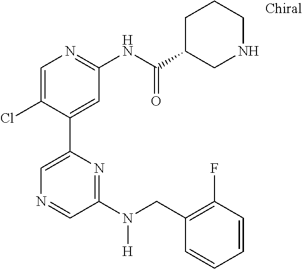 | 441.1 | 0.64 | (R)-Piperidine-3-carboxylic acid {5-chloro-4-[6-(2- fluoro-benzylamino)- pyrazin-2-yl]-pyridin-2-yl}- amide |
| 15 | 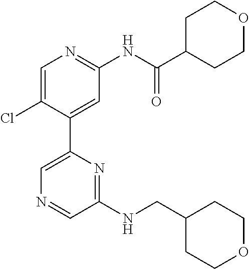 | 390.1 | 0.76 | Tetrahydro-pyran-4- carboxylic acid (5-chloro-4- {6-[(tetrahydro-pyran-4- ylmethyl)-amino]-pyrazin-2- yl}-pyridin-2-yl)-amide |
| 16 | 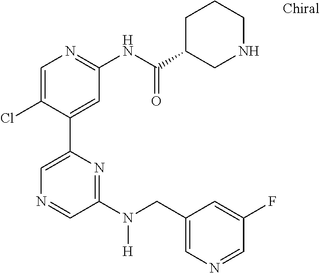 | 442.1 | 0.5 | (R)-Piperidine-3-carboxylic acid (5-chloro-4-{6-[(5- fluoro-pyridin-3-ylmethyl)- amino]-pyrazin-2-yl}- pyridin-2-yl)-amide |
| 17 | 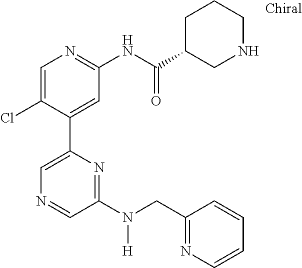 | 424.1 | 0.4 | (R)-Piperidine-3-carboxylic acid (5-chloro-4-{6- [(pyridin-2-ylmethyl)- amino]-pyrazin-2-yl}- pyridin-2-yl)-amide |
| 18 | 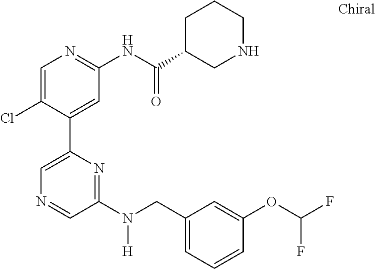 | 489.1 | 0.72 | (R)-Piperidine-3-carboxylic acid {5-chloro-4-[6-(3- difluoromethoxy- benzylamino)-pyrazin-2-yl]- pyridin-2-yl}-amide |
| 19 | 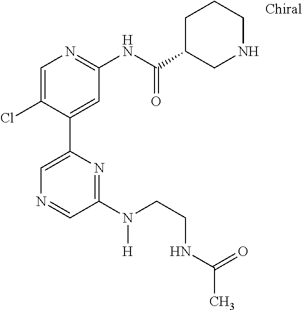 | 418.1 | 0.44 | (R)-Piperidine-3-carboxylic acid {4-[6-(2-acetylamino- ethylamino)-pyrazin-2-yl]-5- chloro-pyridin-2-yl}-amide |
| 20 | 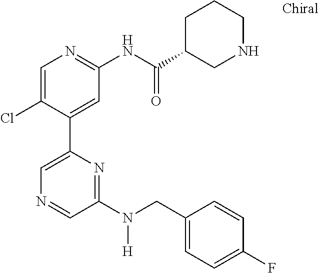 | 441.1 | 0.69 | (R)-Piperidine-3-carboxylic acid {5-chloro-4-[6-(4- fluoro-benzylamino)- pyrazin-2-yl]-pyridin-2-yl}- amide |
| 21 | 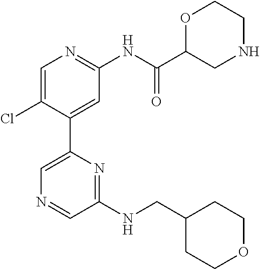 | 433.2 | 0.56 | Morpholine-2-carboxylic acid (5-chloro-4-{6- [(tetrahydro-pyran-4- ylmethyl)-amino]-pyrazin-2- yl}-pyridin-2-yl)-amide |
| 22 | 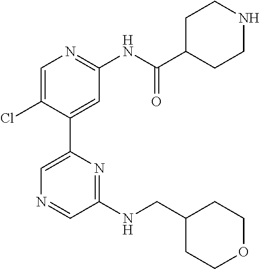 | 431.2 | 0.55 | Piperidine-4-carboxylic acid (5-chloro-4-{6-[(tetrahydro- pyran-4-ylmethyl)-amino]- pyrazin-2-yl}-pyridin-2-yl)- amide |
| 23 | 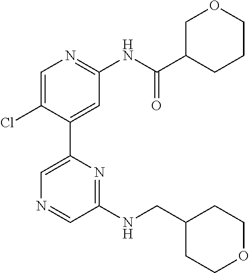 | 432.2 | 0.73 | Tetrahydro-pyran-3- carboxylic acid (5-chloro-4- {6-[(tetrahydro-pyran-4- ylmethyl)-amino]-pyrazin-2- yl}-pyridin-2-yl)-amide |
| 24 | 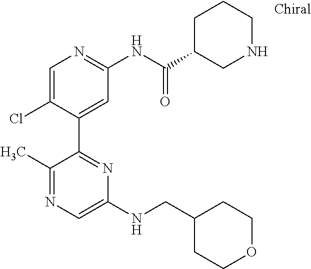 | 445.2 | 0.56 | (R)-Piperidine-3-carboxylic acid (5-chloro-4-{3-methyl- 6-[(tetrahydro-pyran-4- ylmethyl)-amino]-pyrazin-2- yl}-pyridin-2-yl)-amide |
| 25 | 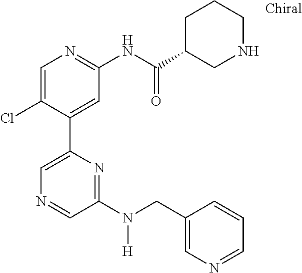 | 424.1 | 0.41 | (R)-Piperidine-3-carboxylic acid (5-chloro-4-{6- [(pyridin-3-ylmethyl)- amino]-pyrazin-2-yl}- pyridin-2-yl)-amide |
| 26 | 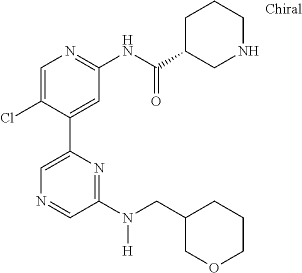 | 431.1 | 0.58 | (R)-Piperidine-3-carboxylic acid (5-chloro-4-{6- [(tetrahydro-pyran-3- ylmethyl)-amino]-pyrazin-2- yl}-pyridin-2-yl)-amide |
| 27 | 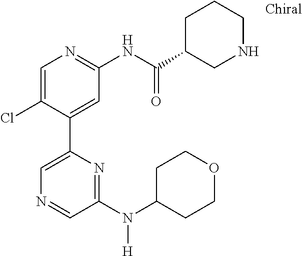 | 417.1 | 0.54 | (R)-Piperidine-3-carboxylic acid {5-chloro-4-[6- (tetrahydro-pyran-4- ylamino)-pyrazin-2-yl]- pyridin-2-yl}-amide |
| 28 | 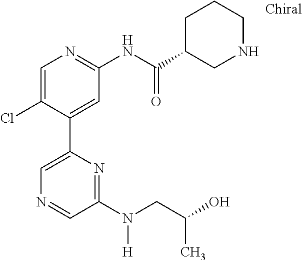 | 391.1 | 0.47 | (R)-Piperidine-3-carboxylic acid {5-chloro-4-[6-((R)-2- hydroxy-propylamino)- pyrazin-2-yl]-pyridin-2-yl}- amide |
| 29 | 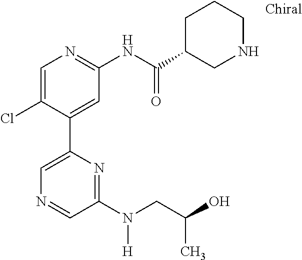 | 391.1 | 0.47 | (R)-Piperidine-3-carboxylic acid {5-chloro-4-[6-((S)-2- hydroxy-propylamino)- pyrazin-2-yl]-pyridin-2-yl}- amide |
| 30 | 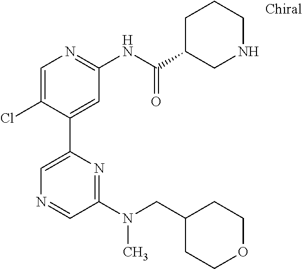 | 445.1 | 0.63 | (R)-Piperidine-3-carboxylic acid (5-chloro-4-{6-[methyl- (tetrahydro-pyran-4- ylmethyl)-amino]-pyrazin-2- yl}-pyridin-2-yl)-amide |
| 31 | 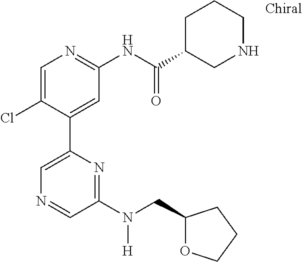 | 417.1 | 0.56 | (R)-Piperidine-3-carboxylic acid [5-chloro-4-(6-{[(R)-1- (tetrahydro-furan-2- yl)methyl]-amino}-pyrazin- 2-yl)-pyridin-2-yl]-amide |
| 32 | 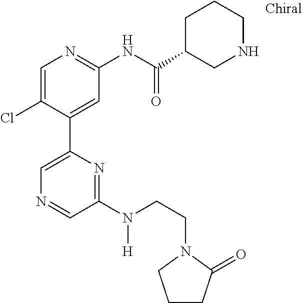 | 444.1 | 0.49 | (R)-Piperidine-3-carboxylic acid (5-chloro-4-{6-[2-(2- oxo-pyrrolidin-1-yl)- ethylamino]-pyrazin-2-yl}- pyridin-2-yl)-amide |
| 33 | 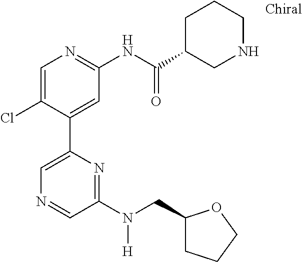 | 417.1 | 0.57 | (R)-Piperidine-3-carboxylic acid [5-chloro-4-(6-{[(S)-1- (tetrahydro-furan-2- yl)methyl]-amino}-pyrazin- 2-yl)-pyridin-2-yl]-amide |
| 34 | 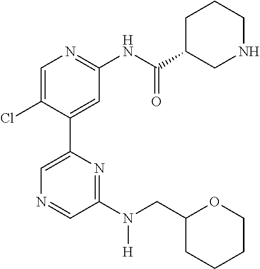 | 431.1 | 0.62 | (R)-Piperidine-3-carboxylic acid (5-chloro-4-{6- [(tetrahydro-pyran-2- ylmethyl)-amino]-pyrazin-2- yl}-pyridin-2-yl)-amide |
| 35 | 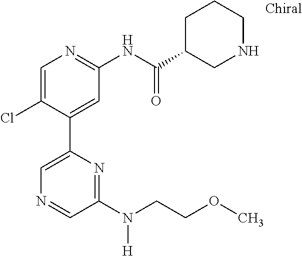 | 391.1 | 0.52 | (R)-Piperidine-3-carboxylic acid {5-chloro-4-[6-(2- methoxy-ethylamino)- pyrazin-2-yl]-pyridin-2-yl}- amide |
| 36 | 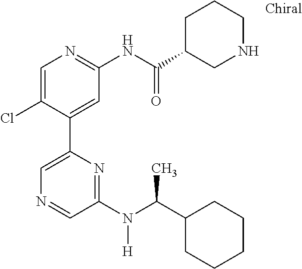 | 443.2 | 0.82 | (R)-Piperidine-3-carboxylic acid {5-chloro-4-[6-((S)-1- cyclohexyl-ethylamino)- pyrazin-2-yl]-pyridin-2-yl}- amide |
| 37 | 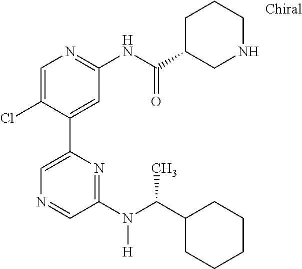 | 443.2 | 0.82 | (R)-Piperidine-3-carboxylic acid {5-chloro-4-[6-((R)-1- cyclohexyl-ethylamino)- pyrazin-2-yl]-pyridin-2-yl}- amide |
| 38 | 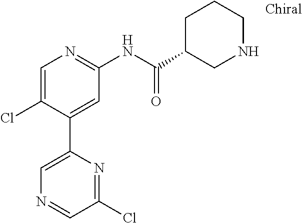 | 352 | 0.61 | (R)-Piperidine-3-carboxylic acid [5-chloro-4-(6-chloro- pyrazin-2-yl)-pyridin-2-yl]- amide |
| 39 | 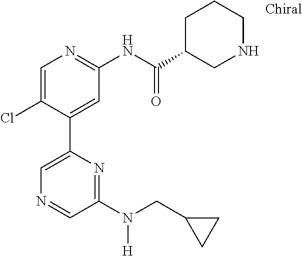 | 387.1 | 0.62 | (R)-Piperidine-3-carboxylic acid {5-chloro-4-[6- (cyclopropylmethyl-amino)- pyrazin-2-yl]-pyridin-2-yl}- amide |
| 40 | 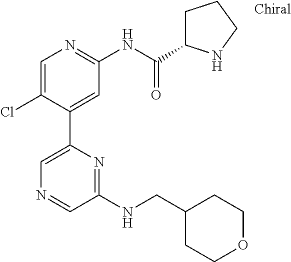 | 417.1 | 0.57 | (S)-Pyrrolidine-2-carboxylic acid (5-chloro-4-{6- [(tetrahydro-pyran-4- ylmethyl)-amino]-pyrazin-2- yl}-pyridin-2-yl)-amide |
| 41 | 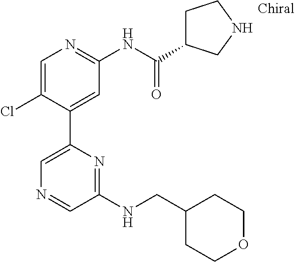 | 417.1 | 0.56 | (R)-Pyrrolidine-3-carboxylic acid (5-chloro-4-{6- [(tetrahydro-pyran-4- ylmethyl)-amino]-pyrazin-2- yl}-pyridin-2-yl)-amide |
| 42 | 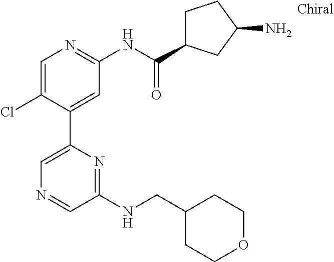 | 431.1 | 0.58 | (1S,3R)-3-Amino- cyclopentanecarboxylic acid (5-chloro-4-{6-[(tetrahydro- pyran-4-ylmethyl)-amino]- pyrazin-2-yl}-pyridin-2-yl)- amide |
| 43 | 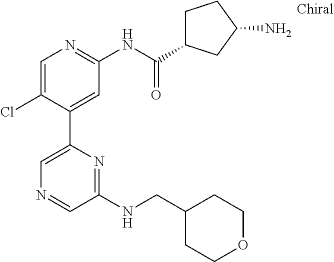 | 431.1 | 0.59 | (1R,3S)-3-Amino- cyclopentanecarboxylic acid (5-chloro-4-{6-[(tetrahydro- pyran-4-ylmethyl)-amino]- pyrazin-2-yl}-pyridin-2-yl)- amide |
| 44 | 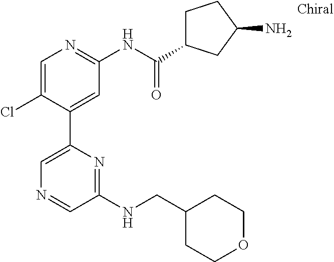 | 431.1 | 0.58 | (1R,3R)-3-Amino- cyclopentanecarboxylic acid (5-chloro-4-{6-[(tetrahydro- pyran-4-ylmethyl)-amino]- pyrazin-2-yl}-pyridin-2-yl)- amide |
| 45 | 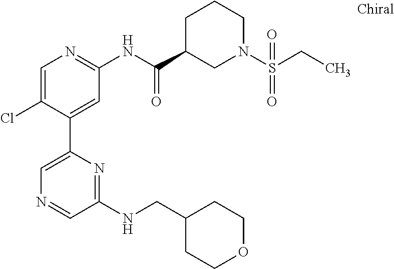 | 523.1 | 0.8 | (S)-1-Ethanesulfonyl- piperidine-3-carboxylic acid (5-chloro-4-{6-[(tetrahydro- pyran-4-ylmethyl)-amino]- pyrazin-2-yl}-pyridin-2-yl)- amide |
| 46 | 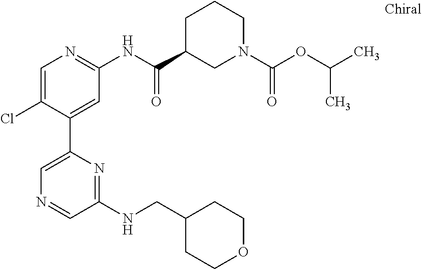 | 517.2 | 0.92 | (S)-3-(5-Chloro-4-{6- [(tetrahydro-pyran-4- ylmethyl)-amino]-pyrazin-2- yl}-pyridin-2-ylcarbamoyl)- piperidine-1-carboxylic acid isopropyl ester |
| 47 | 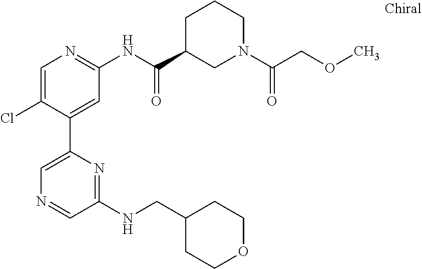 | 503.2 | 0.69 | (S)-1-(2-Methoxy-acetyl)- piperidine-3-carboxylic acid (5-chloro-4-{6-[(tetrahydro- pyran-4-ylmethyl)-amino]- pyrazin-2-yl}-pyridin-2-yl)- amide |
| 48 | 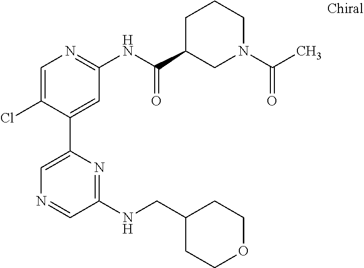 | 473.2 | 0.69 | (S)-1-Acetyl-piperidine-3- carboxylic acid (5-chloro-4- {6-[(tetrahydro-pyran-4- ylmethyl)-amino]-pyrazin-2- yl}-pyridin-2-yl)-amide |
| 49 | 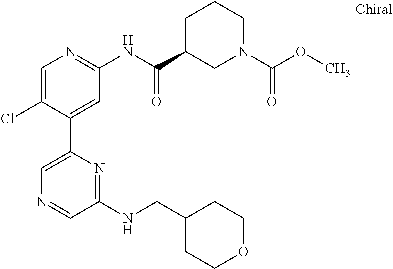 | 489.3 | 0.8 | (S)-3-(5-Chloro-4-{6- [(tetrahydro-pyran-4- ylmethyl)-amino]-pyrazin-2- yl}-pyridin-2-ylcarbamoyl)- piperidine-1-carboxylic acid methyl ester |
| 50 | 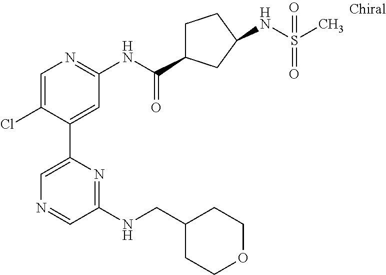 | 509 | 0.71 | (1S,3R)-3- Methanesulfonylamino- cyclopentanecarboxylic acid (5-chloro-4-{6-[(tetrahydro- pyran-4-ylmethyl)-amino]- pyrazin-2-yl}-pyridin-2-yl)- amide |
| 51 | 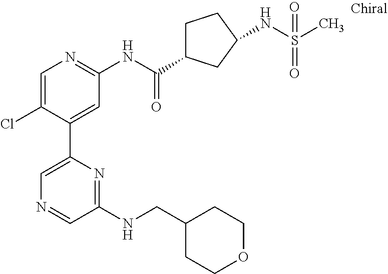 | 509 | 0.69 | (1R,3S)-3- Methanesulfonylamino- cyclopentanecarboxylic acid (5-chloro-4-{6-[(tetrahydro- pyran-4-ylmethyl)-amino]- pyrazin-2-yl}-pyridin-2-yl)- amide |
| 52 | 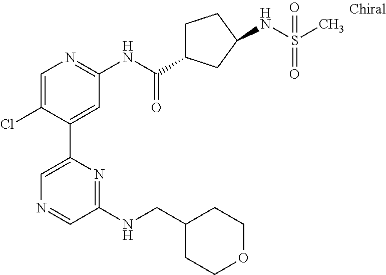 | 509 | 0.69 | (1R,3R)-3- Methanesulfonylamino- cyclopentanecarboxylic acid (5-chloro-4-{6-[(tetrahydro- pyran-4-ylmethyl)-amino]- pyrazin-2-yl}-pyridin-2-yl)- amide |
| 53 | 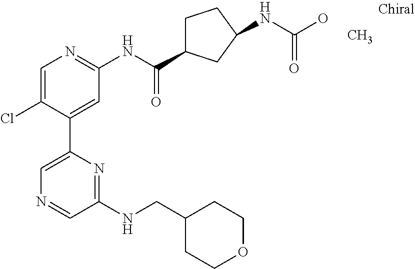 | 489.1 | 0.72 | [(1R,3S)-3-(5-Chloro-4-{6- [(tetrahydro-pyran-4- ylmethyl)-amino]-pyrazin-2- yl}-pyridin-2-ylcarbamoyl)- cyclopentyl]-carbamic acid methyl ester |
| 54 | 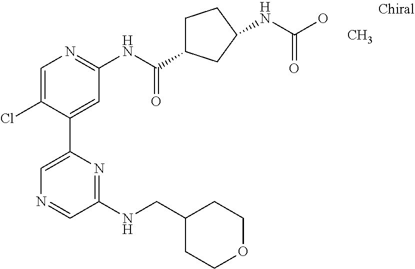 | 489.1 | 0.74 | [(1S,3R)-3-(5-Chloro-4-{6- [(tetrahydro-pyran-4- ylmethyl)-amino]-pyrazin-2- yl}-pyridin-2-ylcarbamoyl)- cyclopentyl]-carbamic acid methyl ester |
| 55 | 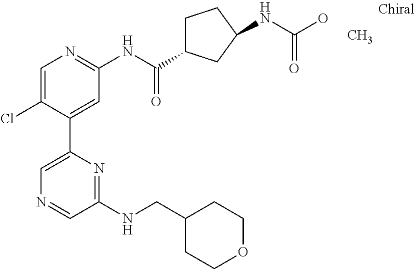 | 489.1 | 0.72 | [(1R,3R)-3-(5-Chloro-4-{6- [(tetrahydro-pyran-4- ylmethyl)-amino]-pyrazin-2- yl}-pyridin-2-ylcarbamoyl)- cyclopentyl]-carbamic acid methyl ester |
| 56 | 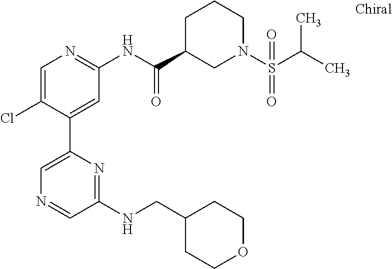 | 537.1 | 0.82 | (S)-1-(Propane-2-sulfonyl)- piperidine-3-carboxylic acid (5-chloro-4-{6-[(tetrahydro- pyran-4-ylmethyl)-amino]- pyrazin-2-yl}-pyridin-2-yl)- amide |
| 57 | 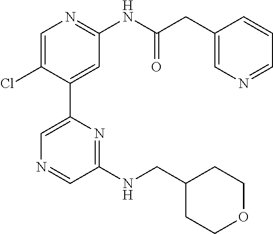 | 439.1 | 0.57 | N-(5-Chloro-4-{6- [(tetrahydro-pyran-4- ylmethyl)-amino]-pyrazin-2- yl}-pyridin-2-yl)-2-pyridin- 3-yl-acetamide |
| 58 | 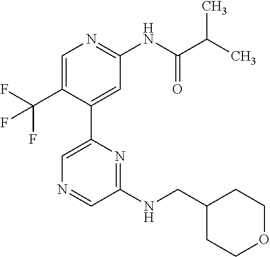 | 424.1 | 0.85 | N-(4-{6-[(Tetrahydro-pyran- 4-ylmethyl)-amino]-pyrazin- 2-yl}-5-trifluoromethyl- pyridin-2-yl)-isobutyramide |
| 59 | 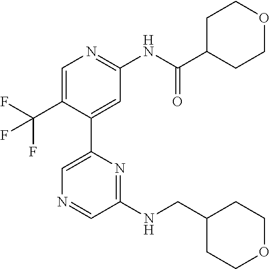 | 466.1 | 0.76 | Tetrahydro-pyran-4- carboxylic acid (4-{6- [(tetrahydro-pyran-4- ylmethyl)-amino]-pyrazin-2- yl}-5-trifluoromethyl- pyridin-2-yl)-amide |
| 60 | 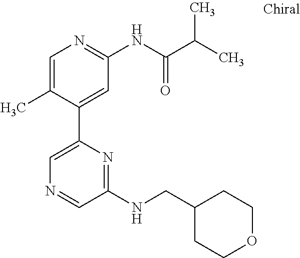 | 370.1 | 0.59 | N-(5-Methyl-4-{6- [(tetrahydro-pyran-4- ylmethyl)-amino]-pyrazin-2- yl}-pyridin-2-yl)- isobutyramide |
| 61 | 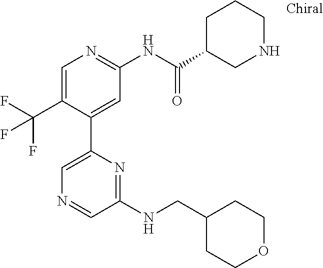 | 465.1 | 0.62 | (R)-Piperidine-3-carboxylic acid (4-{6-[(tetrahydro- pyran-4-ylmethyl)-amino]- pyrazin-2-yl}-5- trifluoromethyl-pyridin-2- yl)-amide |
| 62 | 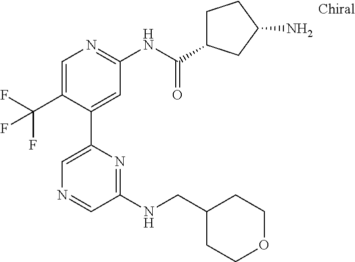 | 465.1 | 0.62 | (1R,3S)-3-Amino- cyclopentanecarboxylic acid (4-{6-[(tetrahydro-pyran-4- ylmethyl)-amino]-pyrazin-2- yl}-5-trifluoromethyl- pyridin-2-yl)-amide |
| 63 | 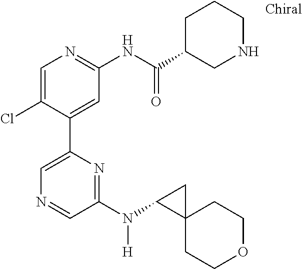 | 443.2 | 0.6 | (R)-Piperidine-3-carboxylic acid (5-chloro-4-{6-[(R)-(6- oxa-spiro[2.5]oct-1- yl)amino]-pyrazin-2-yl}- pyridin-2-yl)-amide |
| 64 | 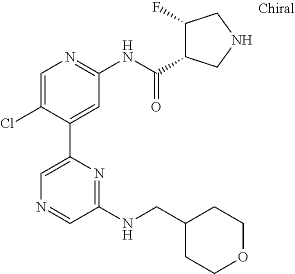 | 435.1 | 0.56 | (3R,4S)-4-Fluoro- pyrrolidine-3-carboxylic acid (5-chloro-4-{6-[(tetrahydro- pyran-4-ylmethyl)-amino]- pyrazin-2-yl}-pyridin-2-yl)- amide |
| 65 | 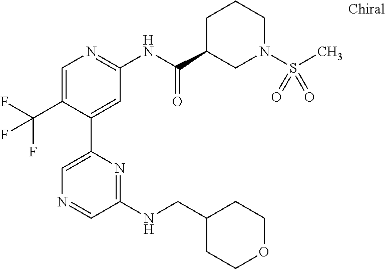 | 543.1 | 0.8 | (S)-1-Methanesulfonyl- piperidine-3-carboxylic acid (4-{6-[(tetrahydro-pyran-4- ylmethyl)-amino]-pyrazin-2- yl}-5-trifluoromethyl- pyridin-2-yl)-amide |
| 66 | 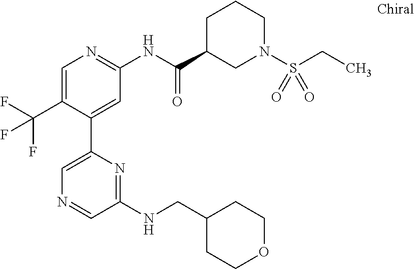 | 557.2 | 0.84 | (S)-1-Ethanesulfonyl- piperidine-3-carboxylic acid (4-{6-[(tetrahydro-pyran-4- ylmethyl)-amino]-pyrazin-2- yl}-5-trifluoromethyl- pyridin-2-yl)-amide |
| 67 | 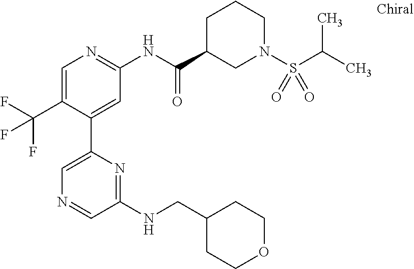 | 571.2 | 0.88 | (S)-1-(Propane-2-sulfonyl)- piperidine-3-carboxylic acid (4-{6-[(tetrahydro-pyran-4- ylmethyl)-amino]-pyrazin-2- yl}-5-trifluoromethyl- pyridin-2-yl)-amide |
| 68 | 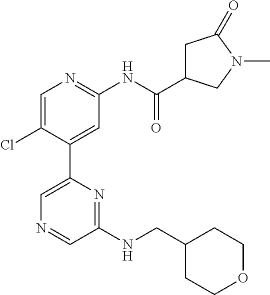 | 445.1 | 0.61 | 1-Methyl-5-oxo-pyrrolidine- 3-carboxylic acid (5-chloro- 4-{6-[(tetrahydro-pyran-4- ylmethyl)-amino]-pyrazin-2- yl}-pyridin-2-yl)-amide |

Table 2 below provides ¹H NMR data for representative compounds.

| Example | |
|---|---|
| No. | ¹H NMR |
| 6 | ¹H NMR (300 MHz, methanol-d4, 25° C.) δ [ppm]: 8.46 (s, 1 H) 8.39 (s, 1 |
| | H), 8.02 (s, 1 H) 7.91 (s, 1 H) 3.95 (dd, J = 11.0, 3.4 Hz, 2 H) 3.85 (dd, J = 11.7, |
| | 1.8 Hz, 1 H) 3.69 (d, J = 11.4 Hz, 1 H) 3.44 (td, J = 11.7, 1.8 Hz, 2 H) |
| | 3.28 - 3.33 (dMeOH, 2 H, App.) 2.88-2.99 (m, 1 H) 2.85 (s, 3 H) 2.70-2.82 |
| | (m, 2 H) 1.82-2.15 (m, 3 H) 1.57-1.80 (m, 4 H) 1.23-1.44 (m, 2 H) |
| 9 | ¹H NMR (300 MHz, methanol-d4, 25° C.) δ [ppm]: 1.23-1.45 (m, J = 12.45, |
| | 12.45, 12.16, 4.54 Hz, 2 H) 1.48-1.66 (m, 1 H) 1.66-1.83 (m, 4 H) 1.94-2.10 |
| | (m, 2 H) 2.55-2.75 (m, 2 H) 2.86 (dd, J = 12.31, 9.67 Hz, 1 H) 2.91-3.04 |
| | (m, 1 H) 3.12 (dd, J = 12.45, 3.08 Hz, 1 H) 3.28-3.34 (dMeOH, 2H |
| | App.) 3.44 (td, J = 11.72, 1.76 Hz, 2 H) 3.95 (dd, J = 11.28, 3.37 Hz, 2 H) 7.90 |
| | (s, 1 H) 8.01 (s, 1 H) 8.38 (s, 1 H) 8.47 (s, 1 H) |
| 15 | ¹H NMR (400 MHz, methanol-d4, 25° C.) δ [ppm]: 8.47 (s, 1 H) 8.37 (s, 1 H) |
| | 8.02 (s, 1 H) 7.91 (s, 1 H) 3.95 (dd, J = 11.2, 3.3 Hz, 2 H) 3.37-3.64 (m, 2 |
| | H) 2.91 (t, J = 7.8 Hz, 1 H) 1.92-2.10 (m, 4 H) 1.58-1.88 (m, 9 H) 1.30-1.39 |
| | (m, 2 H) |
| 37 | ¹H NMR (400 MHz, methanol-d4, 25° C.) δ [ppm]: 8.47 (s, 1 H) 8.40 (s, 1 H) |
| | 7.96 (s, 1 H) 7.89 (s, 1 H) 3.89-4.11 (m, 3 H) 3.33-3.46 (m, 4 H) 3.09-3.26 |
| | (m, 2 H) 2.97-3.09 (m, 1 H) 2.06-2.22 (m, 1 H) 1.89-2.05 (m, 2 H) |
| | 1.64-1.88 (m, 4 H) 1.32-1.61 (m, 2H) 1.21 (d, J = 6.7 Hz, 3 H) |
| 44 | ¹H NMR (400 MHz, methanol-d4, 25° C.) δ [ppm]: 8.38 (s, 1 H) 8.29 (s, 1 H) |
| | 7.92 (s, 1 H) 7.82 (s, 1 H) 3.86 (dd, J = 11.0, 3.5 Hz, 2 H) 3.61-3.73 (m, 1 |
| | H) 3.35-3.48 (m, 2 H) 3.05-3.18 (m, 2 H) 2.32-2.40 (m, 1 H) 2.06-2.26 |
| | (m, 2 H) 1.73-1.99 (m, 4 H) 1.53-1.70 (m, 3 H) 1.18-1.30 (m, 2 H) |
| 47 | ¹H NMR (400 MHz, methanol-d4, 25° C.) δ [ppm]: 8.47 (s, 1 H) 8.39 (s, 1 H) |
| | 8.02 (s, 1 H) 7.91 (s, 1 H) 4.52 (d, J = 11.0 Hz, 1 H) 4.12-4.33 (m, 3 H) |
| | 3.77-4.02 (m, 4 H) 3.37-3.54 (m, 5 H) 3.07-3.17 (m, 1 H) 2.80-3.00 (m, |
| | 1 H) 2.56-2.75 (m, 1 H) 1.94-2.17 (m, 2 H) 1.66-1.89 (m, 4 H) 1.48-1.63 |
| | (m, 1 H) 1.34 (m, 2 H) |
| 64 | ¹H NMR (400 MHz, methanol-d4, 25° C.) δ [ppm]: 8.48 (s, 1 H) 8.42 (s, 1 H) |
| | 8.04 (s, 2H) 7.91 (s, 1 H) 5.57-5.81 (m, 1 H) 3.88-4.06 (m, 4 H) 3.51-3.83 |
| | (m, 5 H) 3.38-3.50 (m, 2 H) 1.94-2.09 (m, 1 H) 1.65-1.78 (m, 2 H) |
| | 1.27-1.43 (m, 2 H) |

Compounds listed in Table 3 below can be made by using the appropriate starting materials and following procedures outlined above.

TABLE 3

| Ex. | |
|---|---|
| No | Structure |
| 101 | 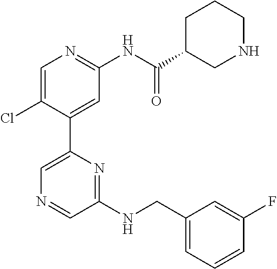 |
| 102 | 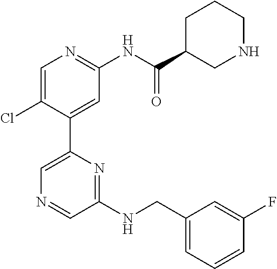 |
| 103 | 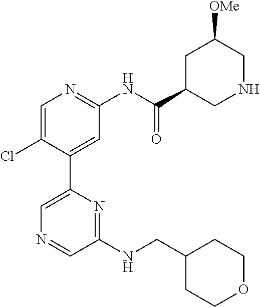 |
| 104 | 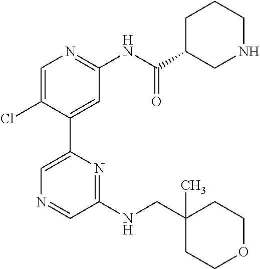 |
| 105 | 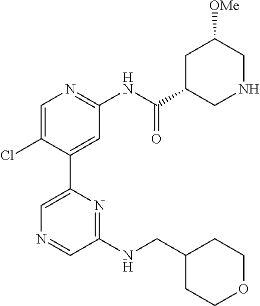 |
| 106 | 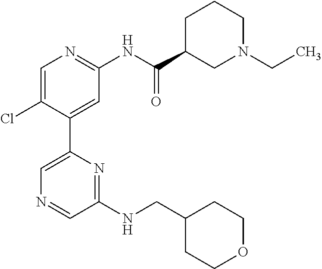 |
| 107 | 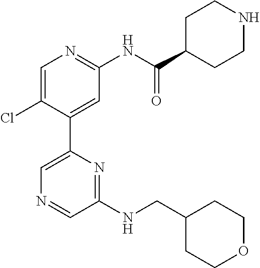 |
| 108 | 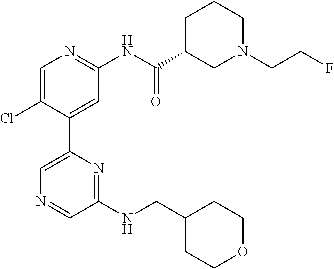 |
| 109 | 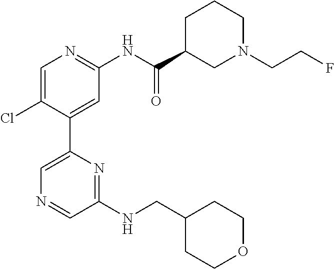 |
| 110 | 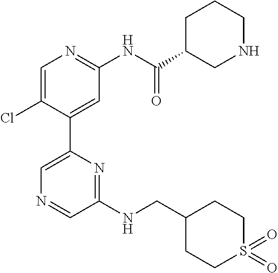 |
| 111 | 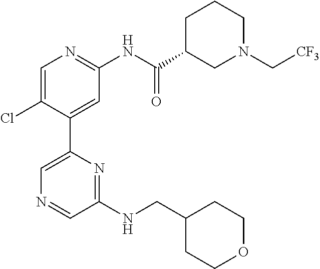 |
| 112 | 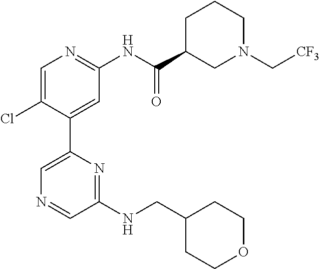 |
| 113 | 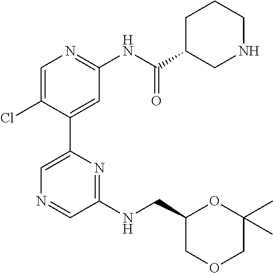 |
| 114 | 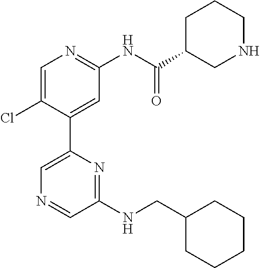 |
| 115 | 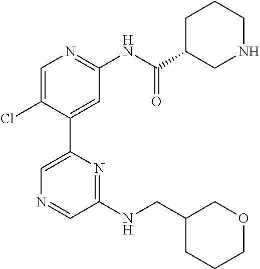 |
| 116 | 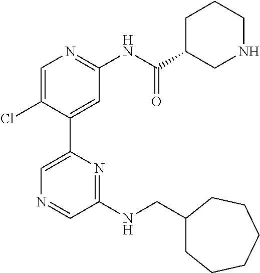 |
| 117 | 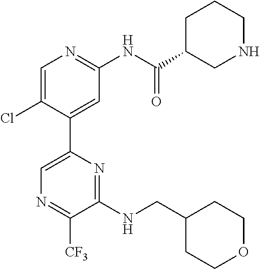 |
| 118 | 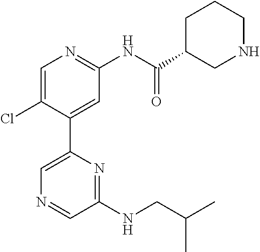 |
| 119 | 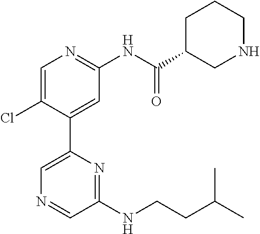 |
| 120 | 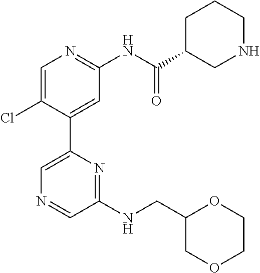 |
| 121 | 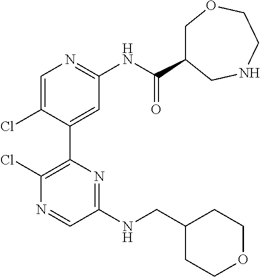 |
| 122 | 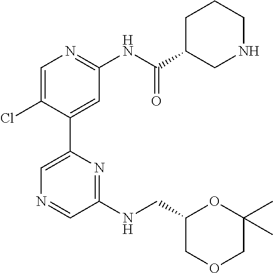 |
| 123 | 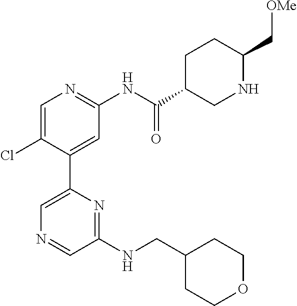 |
| 124 | 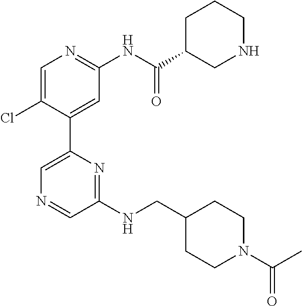 |
| 125 | 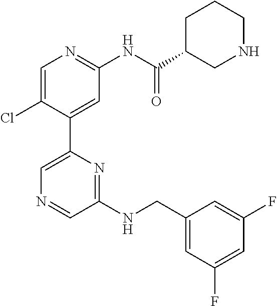 |
| 126 | 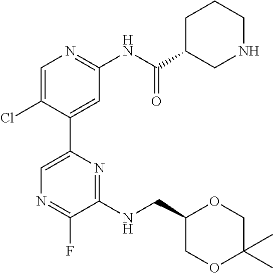 |
| 127 | 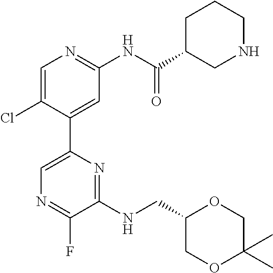 |
| 128 | 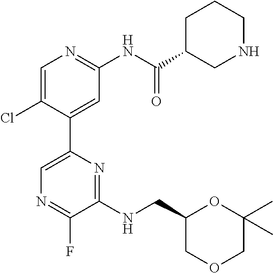 |
| 129 | 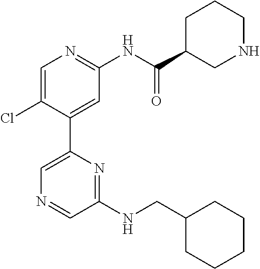 |
| 130 | 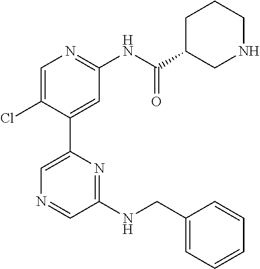 |
| 131 | 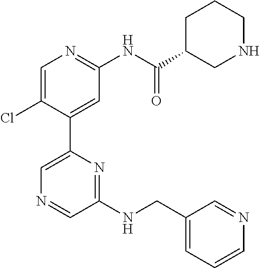 |
| 132 | 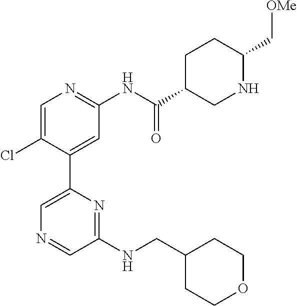 |
| 133 | 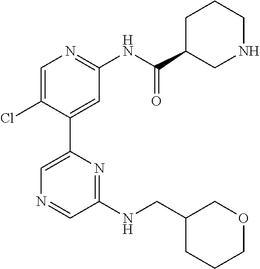 |
| 134 | 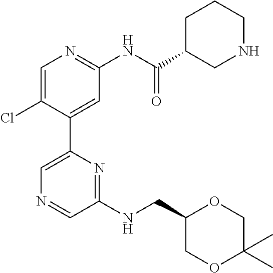 |
| 135 | 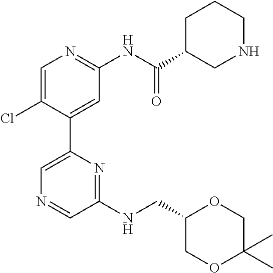 |
| 136 | 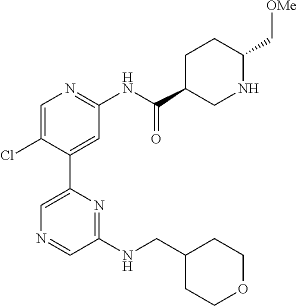 |
| 137 | 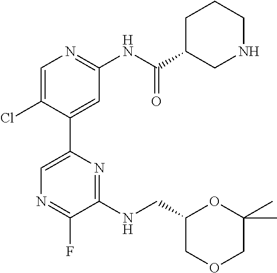 |
| 138 | 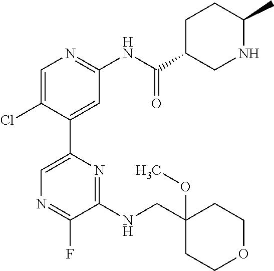 |
| 139 | 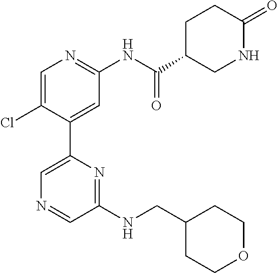 |
| 140 | 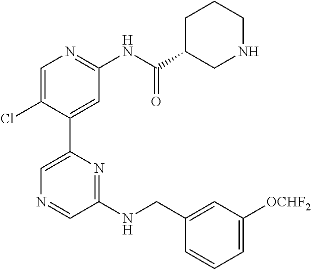 |
| 141 | 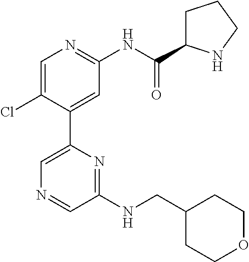 |
| 142 | 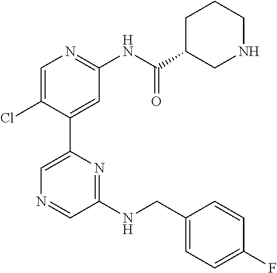 |
| 143 | 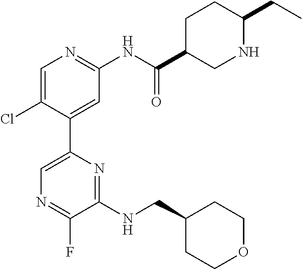 |
| 144 | 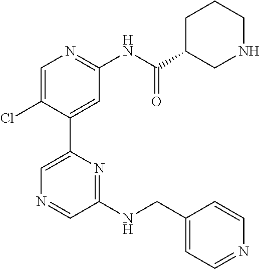 |
| 145 | 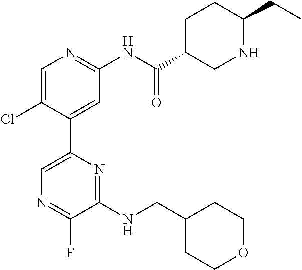 |
| 146 | 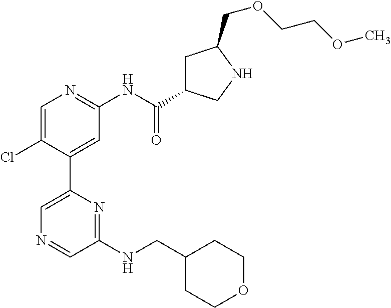 |
| 147 | 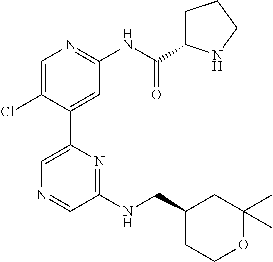 |
| 148 | 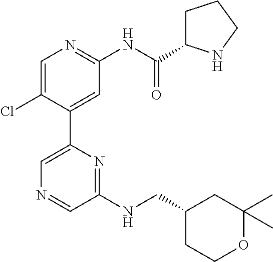 |
| 149 | 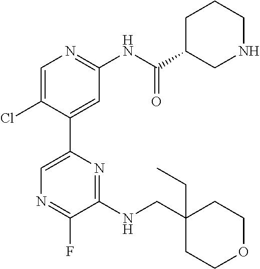 |
| 150 | 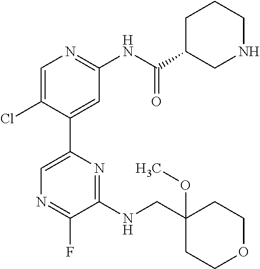 |
| 151 | 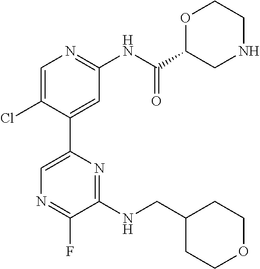 |
| 152 | 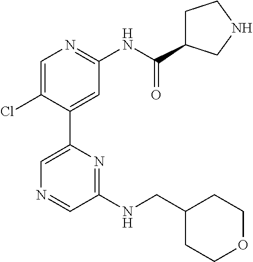 |
| 153 | 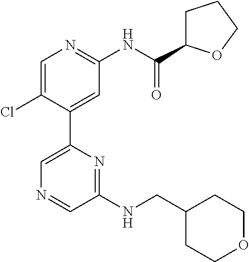 |
| 154 | 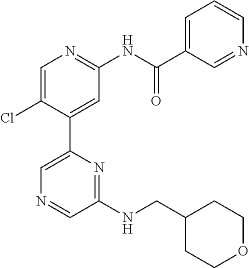 |
| 155 | 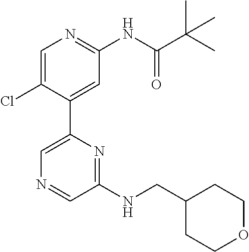 |
| 156 | 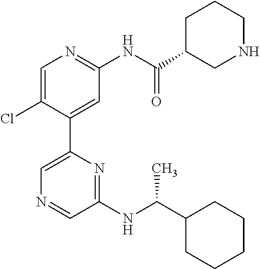 |
| 157 | 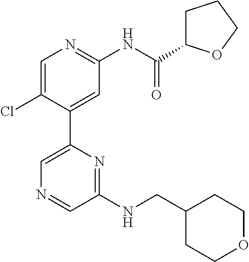 |
| 158 | 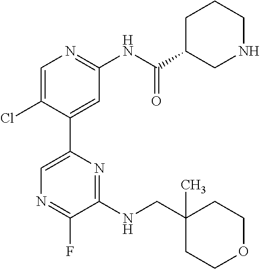 |
| 159 | 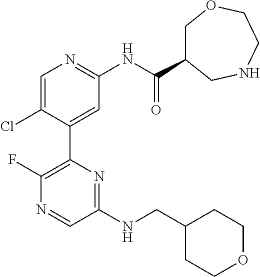 |
| 160 | 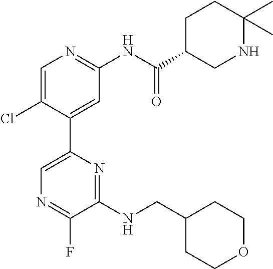 |
| 161 | 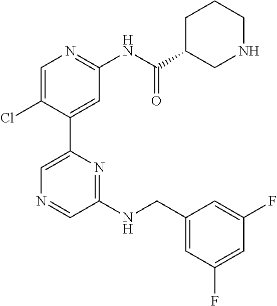 |
| 162 | 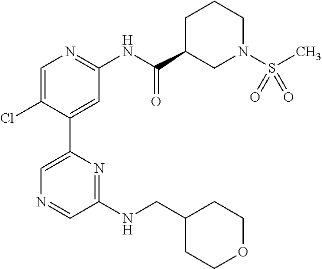 |
| 163 | 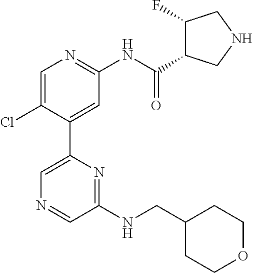 |
| 164 | 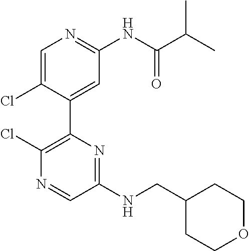 |
| 165 | 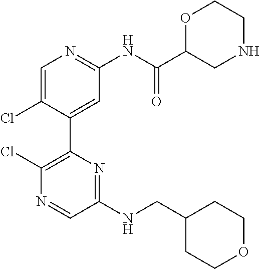 |
| 166 | 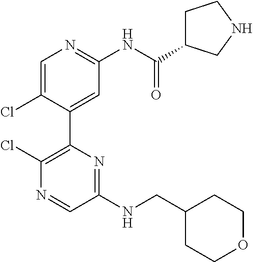 |
| 167 | 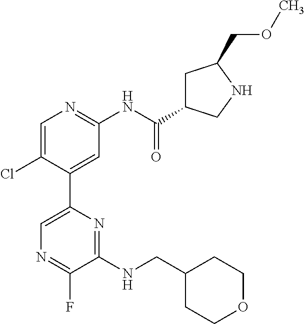 |
| 168 | 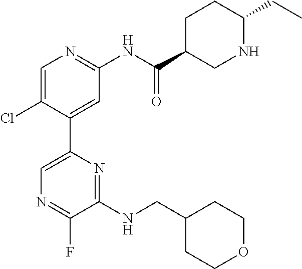 |
| 169 | 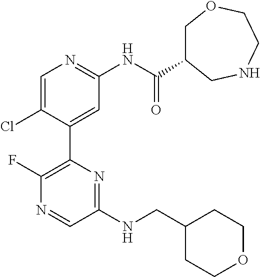 |
| 170 | 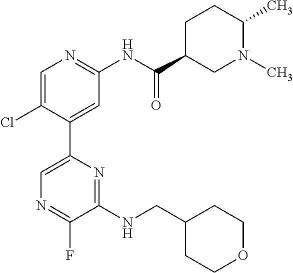 |
| 171 | 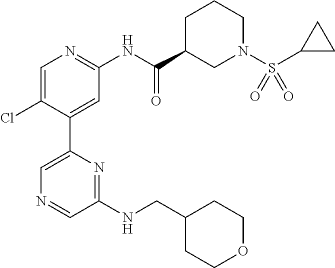 |
| 172 | 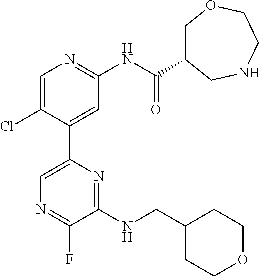 |
| 173 | 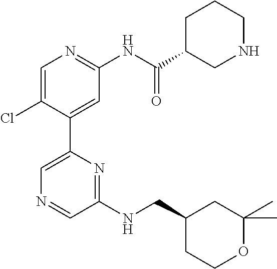 |
| 174 | 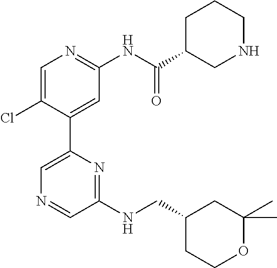 |
| 175 | 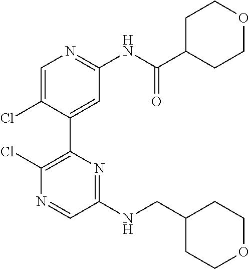 |
| 176 | 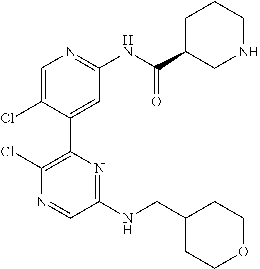 |
| 177 | 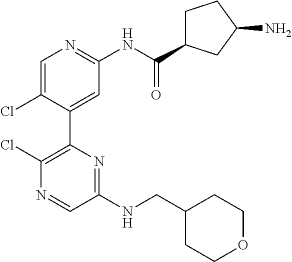 |
| 178 | 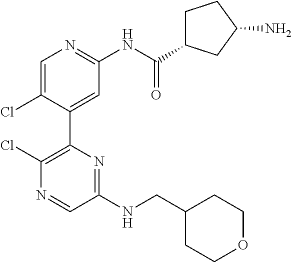 |
| 179 | 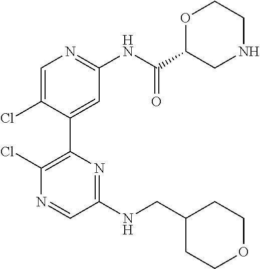 |
| 180 | 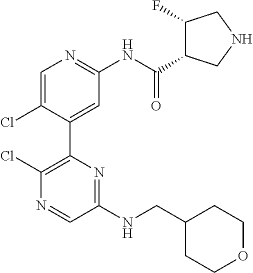 |
| 181 | 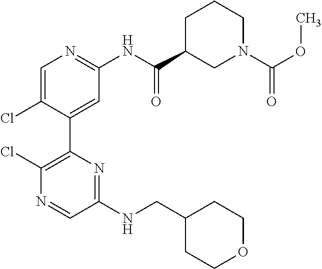 |
| 182 | 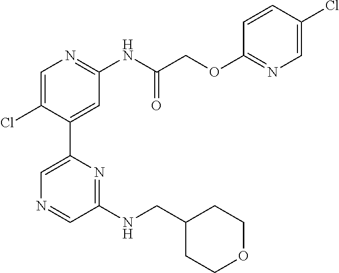 |
| 183 | 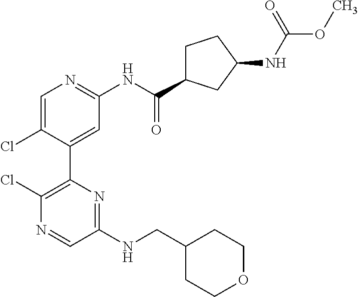 |
| 184 | 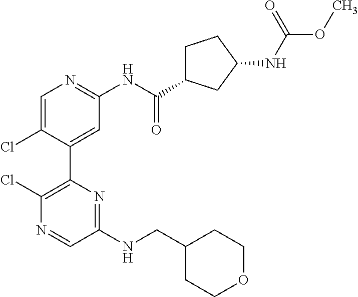 |
| 185 | 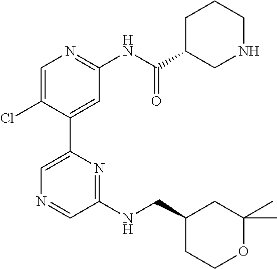 |
| 186 | 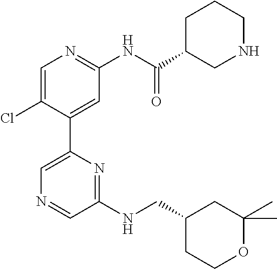 |
| 187 | 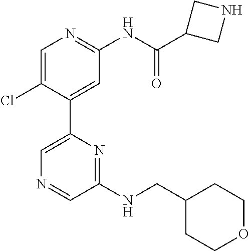 |
| 188 | 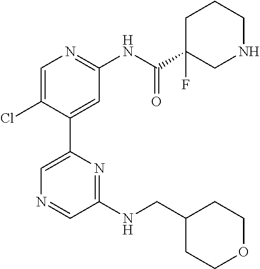 |
| 189 | 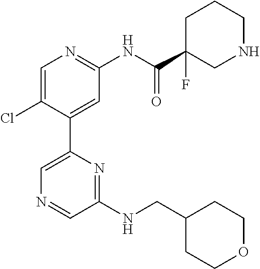 |
| 190 | 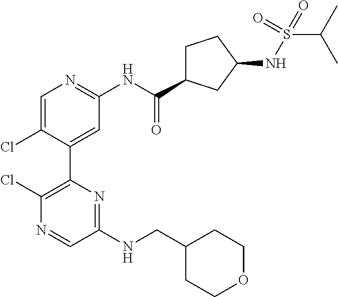 |
| 191 | 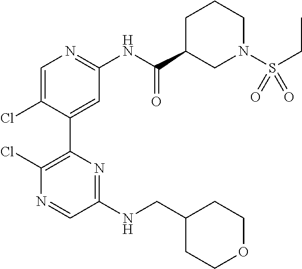 |
| 192 | 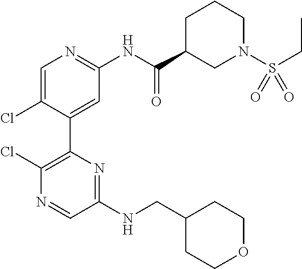 |
| 193 | 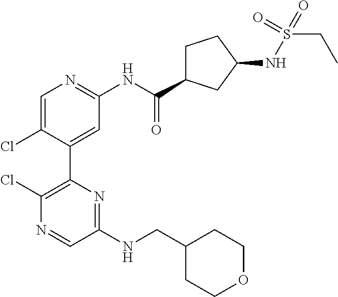 |
| 194 | 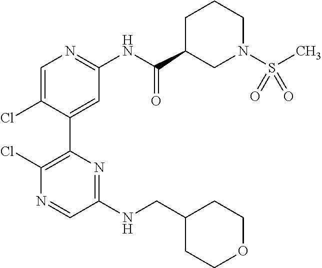 |
| 195 | 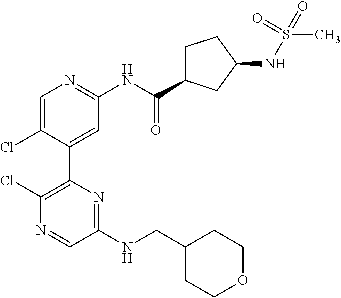 |
| 196 | 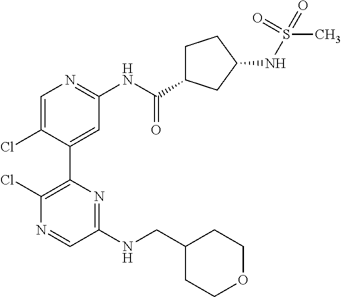 |
| 197 | 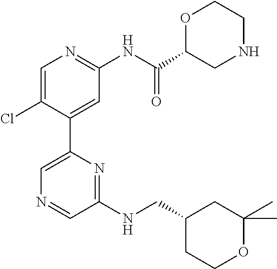 |
| 198 | 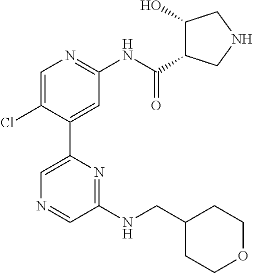 |
| 199 | 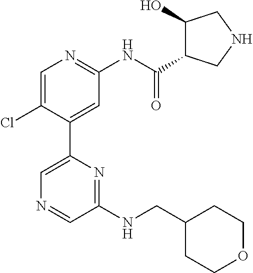 |
| 200 | 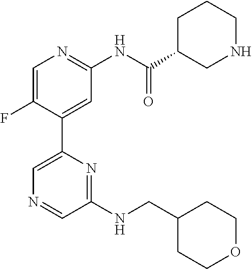 |
| 201 | 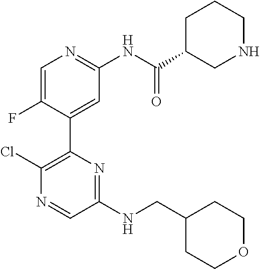 |
| 202 | 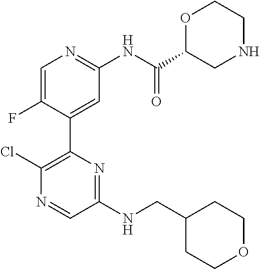 |
| 203 | 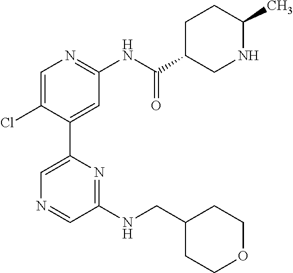 |
| 204 | 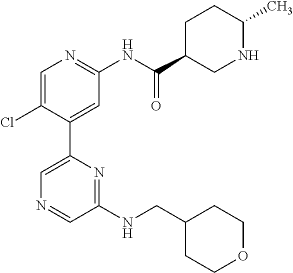 |
| 205 | 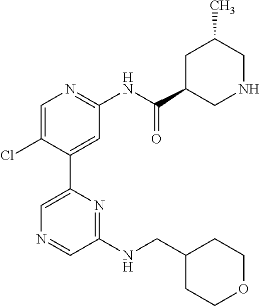 |
| 206 | 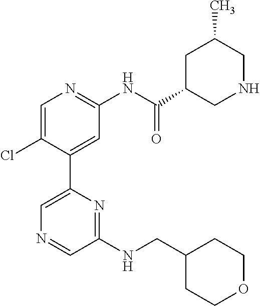 |
| 207 | 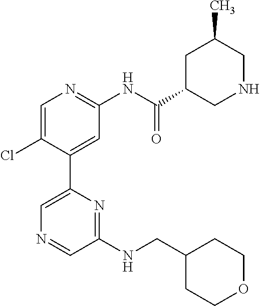 |
| 208 | 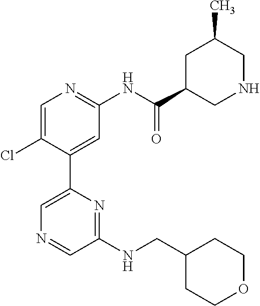 |
| 209 | 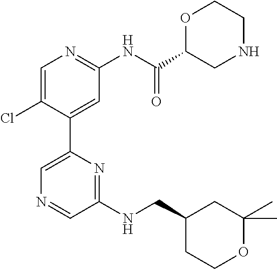 |
| 210 | 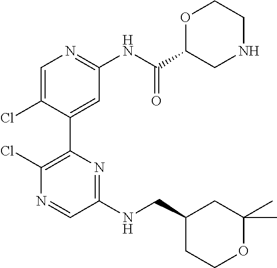 |
| 211 | 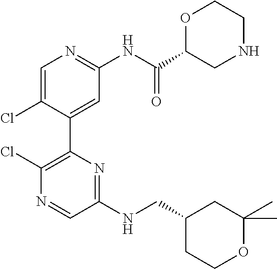 |
| 212 | 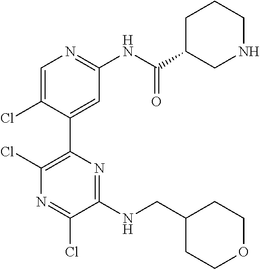 |
| 213 | 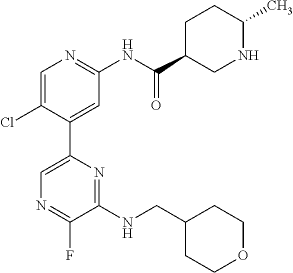 |
| 214 | 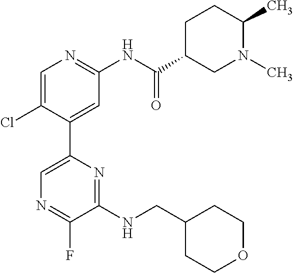 |
| 215 | 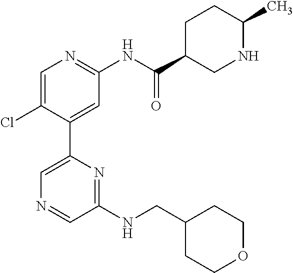 |
| 216 | 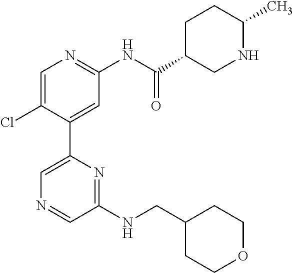 |
| 217 | 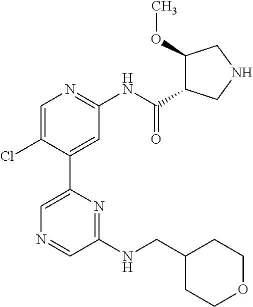 |
| 218 | 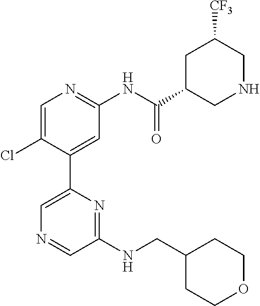 |
| 219 | 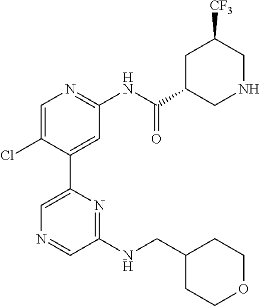 |
| 220 | 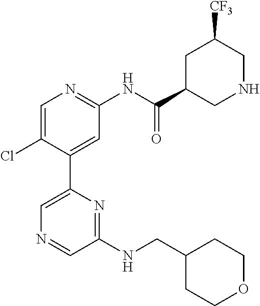 |
| 221 | 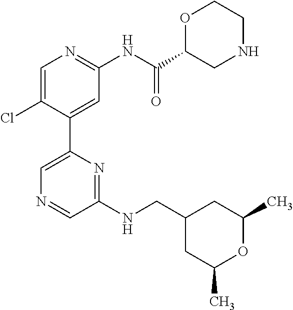 |
| 222 | 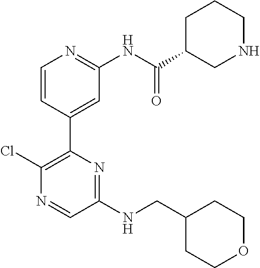 |
| 223 | 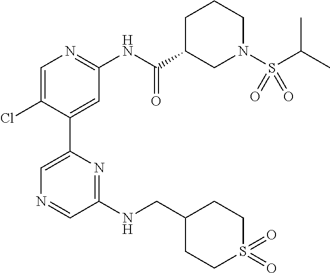 |
| 224 | 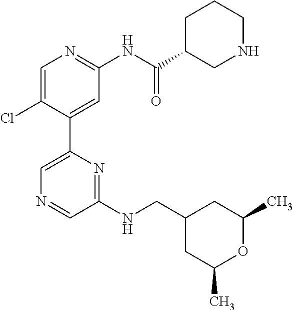 |
| 225 | 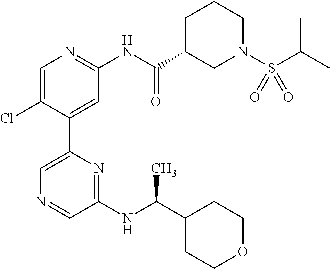 |
| 226 | 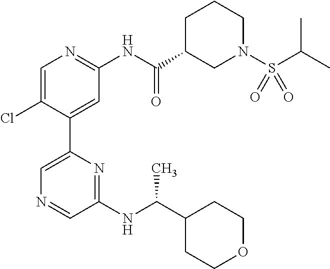 |
| 227 | 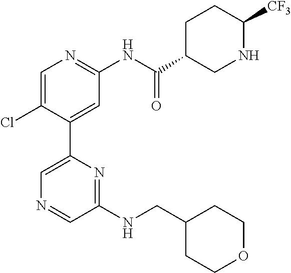 |
| 228 | 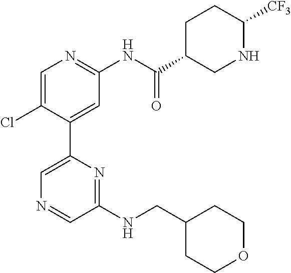 |
| 229 | 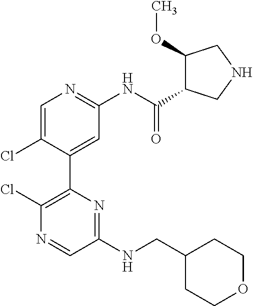 |
| 230 | 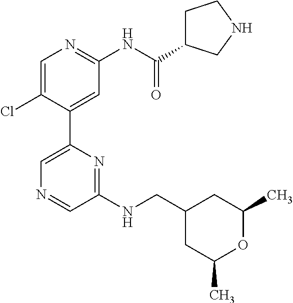 |
| 231 | 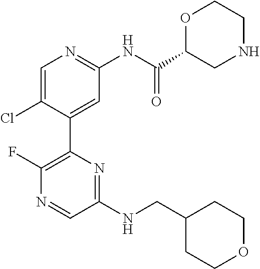 |
| 232 | 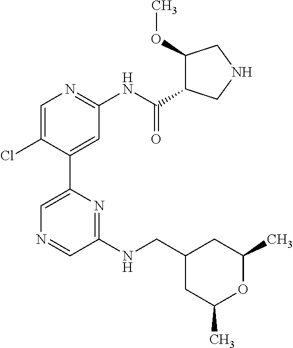 |
| 233 | 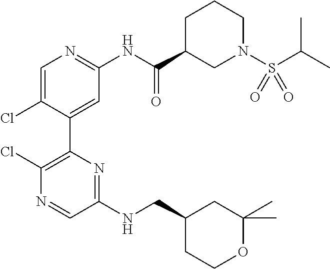 |
| 234 | 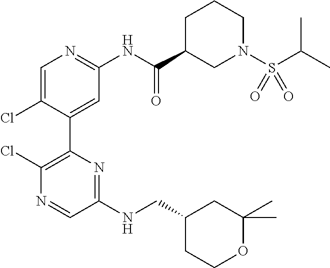 |
| 235 | 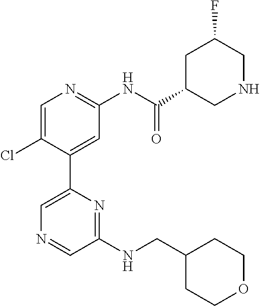 |
| 236 | 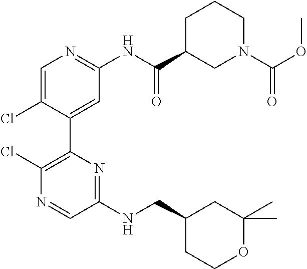 |
| 237 | 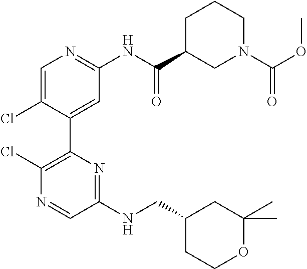 |
| 238 | 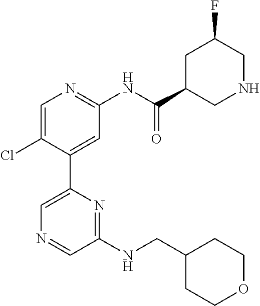 |
| 239 | 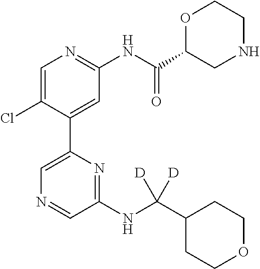 |
| 240 | 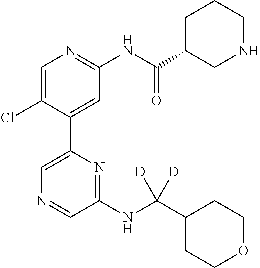 |
| 241 | 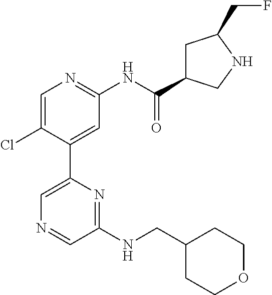 |
| 242 | 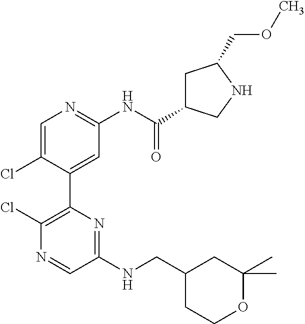 |
| 243 | 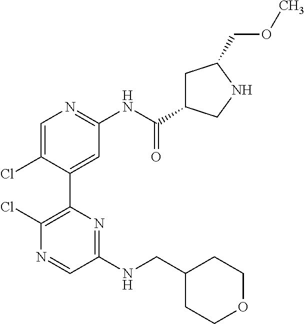 |
| 244 | 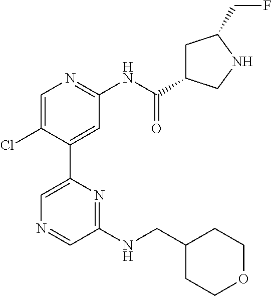 |
| 245 | 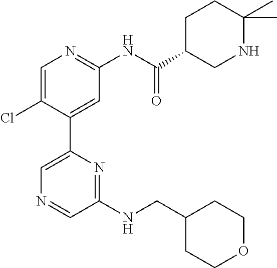 |
| 246 | 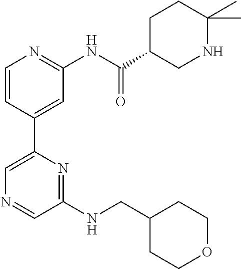 |
| 247 | 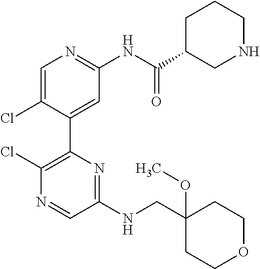 |
| 248 | 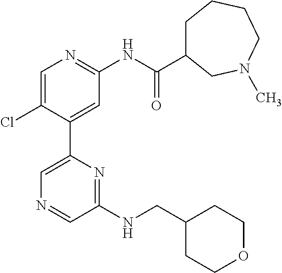 |
| 249 | 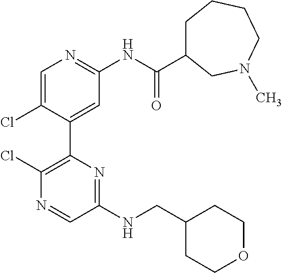 |
| 250 | 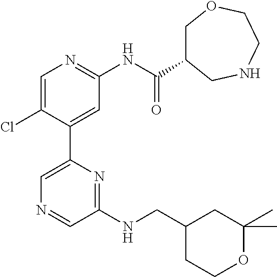 |
| 251 | 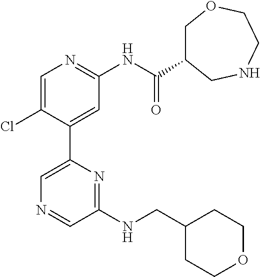 |
| 252 | 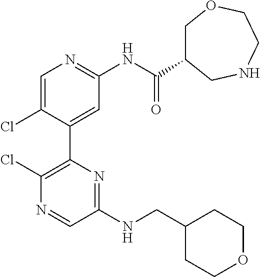 |
| 253 | 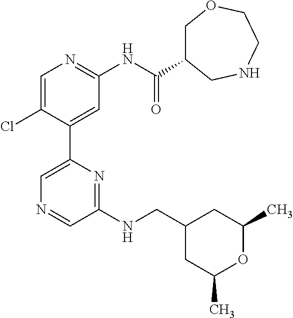 |
| 254 | 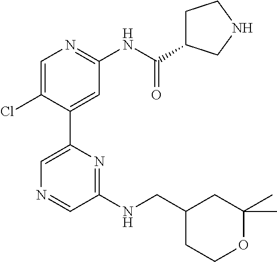 |
| 255 | 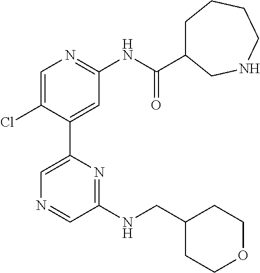 |
| 256 | 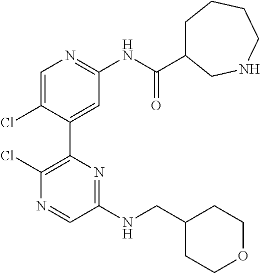 |
| 257 | 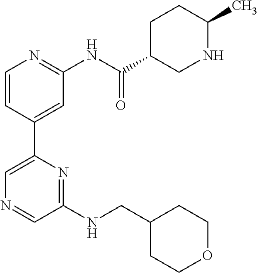 |
| 258 | 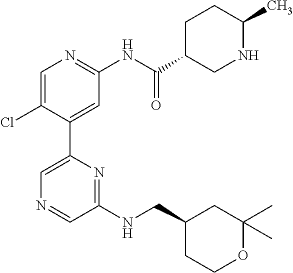 |
| 259 | 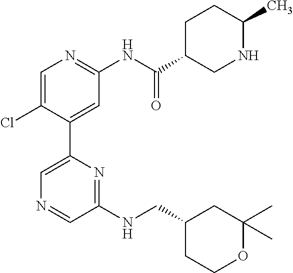 |
| 260 | 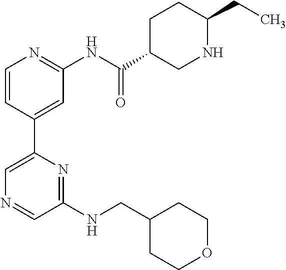 |
| 261 | 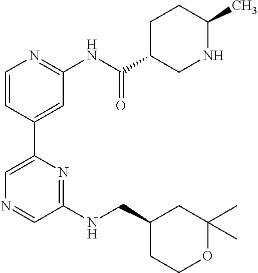 |
| 262 | 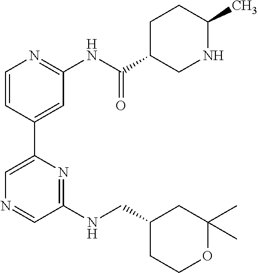 |
| 263 | 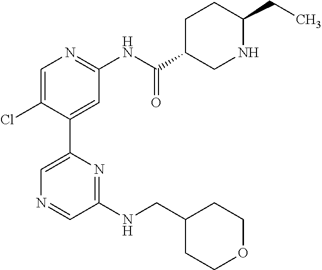 |
| 264 | 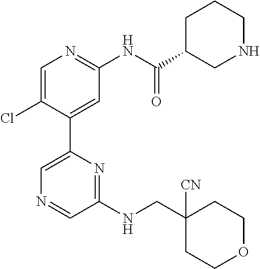 |
| 265 | 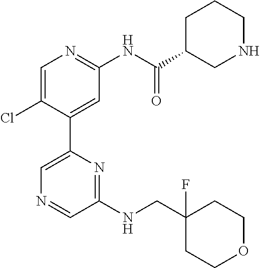 |
| 266 | 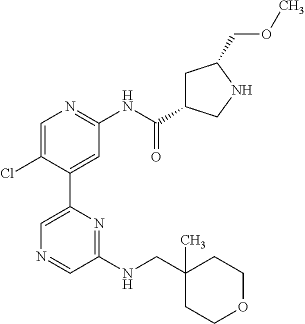 |
| 267 | 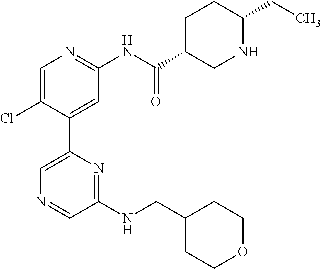 |
| 268 | 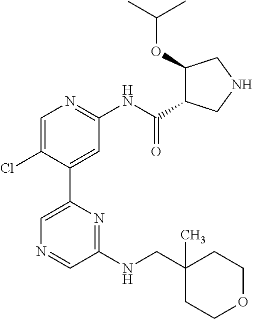 |
| 269 | 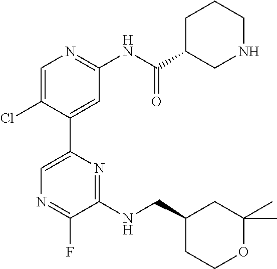 |
| 270 | 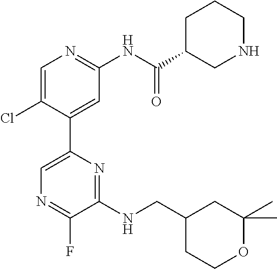 |
| 271 | 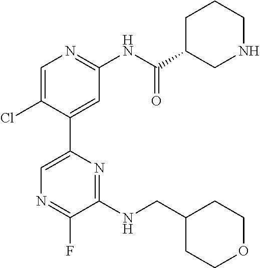 |
| 272 | 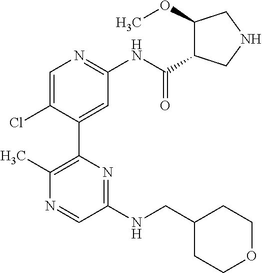 |
| 273 | 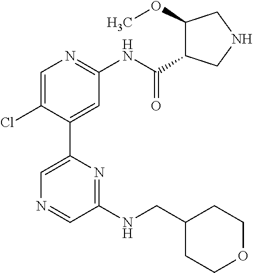 |
| 274 | 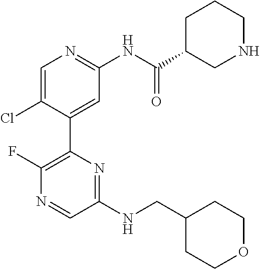 |
| 275 | 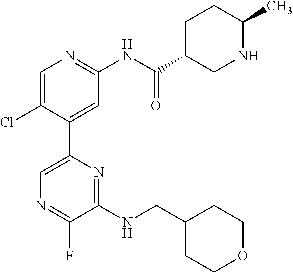 |
| 276 | 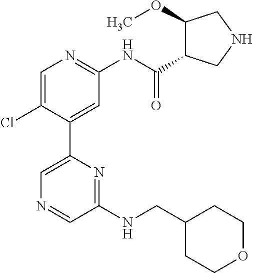 |
| 277 | 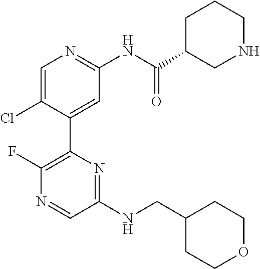 |
| 278 | 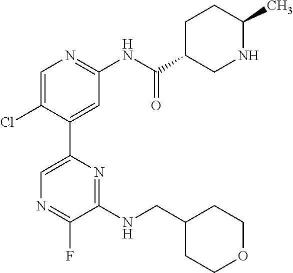 |

### Biological Methods

Cdk9/cyclinT1 IMAP Protocol

The biological activity of the compounds of the invention can be determined using the assay described below.

Cdk9/cyclinT1 is purchased from Millipore, cat #14-685. The final total protein concentration in the assay 4 nM. The 5TAMRA-cdk7tide peptide substrate, 5TAMRA-YSPTSPSYSPTSPSYSTPSPS-COOH, is purchased from Molecular Devices, cat#R7352. The final concentration of peptide substrate is 100 nM. The ATP substrate (Adenosine-5′-triphosphate) is purchased from Roche Diagnostics, cat#1140965. The final concentration of ATP substrate is 6 uM. IMAP (Immobilized Metal Assay for Phosphochemicals) Progressive Binding reagent is purchased from Molecular Devices, cat#R8139. Fluorescence polarization (FP) is used for detection. The 5TAMRA-cdk7tide peptide is phosphorylated by Cdk9/cyclinT1 kinase using the ATP substrate. The Phospho-5TAMRA-cdk7tide peptide substrate is bound to the IMAP Progressive Binding Reagent. The binding of the IMAP Progressive Binding Reagent changes the fluorescence polarization of the 5TAMRA-cdk7tide peptide which is measured at an excitation of 531 nm and FP emission of 595 nm. Assays are carried out in 100 mM Tris, pH=7.2, 10 mM MgCl₂, 0.05% NaN₃, 0.01% Tween-20, 1 mM dithiothreitol and 2.5% dimethyl sulfoxide. IMAP Progressive Binding Reagent is diluted 1:800 in 100% 1× Solution A from Molecular Devices, cat#R7285.

General protocol is as follows: To 10 uL of cdk9/cyclinT1, 0.5 uL of test compound in dimethyl sulfoxide is added. 5TAMRA-cdk7tide and ATP are mixed. 10 uL of the 5TAMRA-cdk7tide/ATP mix is added to start the reaction. The reaction is allowed to proceed for 4.5 hrs. 60 uL of IMAP Progressive Binding Reagent is added. After >1 hr of incubation, plates are read on the Envision 2101 from Perkin-Elmer. The assay is run in a 384-well format using black Corning plates, cat#3573.

Cdk9/cyclinT1 Alpha Screen Protocol

Full length wild type Cdk9/cyclin T1 is purchased from Invitrogen, cat#PV4131. The final total protein concentration in the assay 1 nM. The cdk7tide peptide substrate, biotin-GGGGYSPTSPSYSPTSPSYSPTSPS-OH, is a custom synthesis purchased from the Tufts University Core Facility. The final concentration of cdk7tide peptide substrate is 200 nM. The ATP substrate (Adenosine-5′-triphosphate) is purchased from Roche Diagnostics. The final concentration of ATP substrate is 6 uM. Phospho-Rpb1 CTD (ser2/5) substrate antibody is purchased from Cell Signaling Technology. The final concentration of antibody is 0.67 ug/mL. The Alpha Screen Protein A detection kit containing donor and acceptor beads is purchased from PerkinElmer Life Sciences. The final concentration of both donor and acceptor beads is 15 ug/mL. Alpha Screen is used for detection. The biotinylated-cdk7tide peptide is phosphorylated by cdk9/cyclinT1 using the ATP substrate. The biotinylated-cdk7tide peptide substrate is bound to the streptavidin coated donor bead. The antibody is bound to the protein A coated acceptor bead. The antibody will bind to the phosphorylated form of the biotinylated-cdk7tide peptide substrate, bringing the donor and acceptor beads into close proximity. Laser irradiation of the donor bead at 680 nm generates a flow of short-lived singlet oxygen molecules. When the donor and acceptor beads are in close proximity, the reactive oxygen generated by the irradiation of the donor beads initiates a luminescence/fluorescence cascade in the acceptor beads. This process leads to a highly amplified signal with output in the 530-620 nm range. Assays are carried out in 50 mM Hepes, pH=7.5, 10 mM MgCl₂, 0.1% Bovine Serum Albumin, 0.01% Tween-20, 1 mM Dithiolthreitol, 2.5% Dimethyl Sulfoxide. Stop and detection steps are combined using 50 mM Hepes, pH=7.5, 18 mM EDTA, 0.1% Bovine Serum Albumin, 0.01% Tween-20. General protocol is as follows: To 5 uL of cdk9/cyclinT1, 0.25 uL of test compound in dimethyl sulfoxide is added. Cdk7tide peptide and ATP are mixed. 5 uL of the cdk7tide peptide/ATP mix is added to start the reaction. The reaction is allowed to proceed for 5 hrs. 10 uL of Ab/Alpha Screen beads/Stop-detection buffer is added. Care is taken to keep Alpha Screen beads in the dark at all times. Plates are incubated at room temperature overnight, in the dark, to allow for detection development before being read. The assay is run is a 384-well format using white polypropylene Greiner plates.

The data shown in Table 4 below were generated using one of the assays described above.

TABLE 4

| Ex. | |
|---|---|
| No. | Cdk9_cyclinT1_IC₅₀ [μM] |
| 1 | 2.099 |
| 2 | 0.016 |
| 3 | 0.316 |
| 4 | 0.179 |
| 5 | 0.268 |
| 6 | <0.008 |
| 7 | 0.229 |
| 8 | 0.028 |
| 9 | <0.008 |
| 10 | 0.124 |
| 11 | 0.029 |
| 12 | <0.008 |
| 13 | 0.015 |
| 14 | <0.008 |
| 15 | <0.008 |
| 16 | <0.008 |
| 17 | 0.121 |
| 18 | <0.008 |
| 19 | 0.153 |
| 20 | <0.008 |
| 21 | 0.0128 |
| 22 | 0.008 |
| 23 | 0.009 |
| 24 | <0.008 |
| 25 | 0.0120 |
| 26 | <0.008 |
| 27 | 0.048 |
| 28 | 0.015 |
| 29 | 0.037 |
| 30 | 0.012 |
| 31 | 0.019 |
| 32 | 0.020 |
| 33 | 0.015 |
| 34 | 0.011 |
| 35 | 0.047 |
| 36 | <0.008 |
| 37 | <0.008 |
| 38 | 1.399 |
| 39 | 0.017 |
| 40 | 0.037 |
| 41 | <0.008 |
| 42 | 0.025 |
| 43 | <0.008 |
| 44 | <0.008 |
| 45 | <0.008 |
| 46 | <0.008 |
| 47 | <0.008 |
| 48 | <0.008 |
| 49 | <0.008 |
| 50 | <0.008 |
| 51 | <0.008 |
| 52 | <0.008 |
| 53 | <0.008 |
| 54 | <0.008 |
| 55 | <0.008 |
| 56 | <0.008 |
| 57 | <0.008 |
| 58 | 0.146 |
| 59 | 1.007 |
| 60 | 0.073 |
| 61 | 0.130 |
| 62 | 0.040 |
| 63 | 0.047 |
| 64 | <0.008 |
| 65 | 0.103 |
| 66 | 0.100 |
| 67 | 0.133 |
| 68 | <0.008 |

1. A compound of Formula I 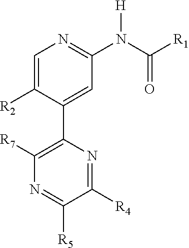
or a pharmaceutically acceptable salt thereof, wherein:
R₁ is selected from —(CH₂)₀₋₂-heteroaryl, —(CH₂)₀₋₂-aryl, C₃₋₈ cycloalkyl, and a 4 to 8 membered heterocycloalkyl group, wherein said groups are each independently optionally substituted;
R₂ is selected from, C₁₋₄ alkoxy, C₁₋₄ haloalkyl, C₁₋₄-alkyl, and halogen;
R₄ is selected from 5 to 7 membered heterocyclyl-R¹⁴, and A₆-L-R₉;
R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;
R₇ is selected from hydrogen, C₁₋₄ alkyl, C₁₋₄ haloalkyl, O—C₁₋₃ alkyl, and halogen;
A₆ is selected from O and NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, and C₃₋₈ branched haloalkylene;
R₈ is selected from hydrogen, C₁₋₄ alkyl, C₃₋₈ branched-alkyl, and —C₃₋₈ branched haloalkyl;
R₉ is selected from C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂— aryl, wherein said groups are optionally substituted; and
R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl.

2. A compound of claim 1, wherein:
R₁ is selected from —(CH₂)₀₋₂-heteroaryl, and —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, cyclo alkyl, —NH—C₃₋₇ cyclo haloalkyl, —NH—(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cycle haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cycle alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —C(O)—C₃₋₇ cycle haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

3. A compound of claim 1, wherein:
R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆ heterocycloalkyl-R¹⁴, —SO₂—NR¹⁵R¹⁶ and —SO₂—C₃₋₅ cycloalkyl;
R₂ is halogen;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, and CF₃;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;
R¹⁴ selected from phenyl, halogen, hydroxyl, C₁₋₂-alkyl, CF₃, and hydrogen; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

4. A compound of claim 1, wherein:
R₁ is selected from C₃₋₈ cycloalkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cycle alkyl, —C₃₋₇ cycle haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cycle haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cycle alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cycle alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₄ branched haloalkyl, NH—SO₂—C₃₋₆ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cycle haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cycle haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cycle haloalkyl, —C(O)—NR¹⁵R¹⁶, and —SO₂—NR¹⁵R¹⁶, and further wherein, any two said substituents along with the atoms to which they are attached can form a ring;
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

5. A compound of claim 1, wherein:
R₁ is selected from C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—Cl₁₋₂ alkyl, —O—C₁₋₄ alkyl, —C(O)O—C₃₋₆ branched alkyl, —C(O)C₁₋₄ alkyl, C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;
R₂ is halogen;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, methyl, and CF₃;
R₇ is selected from hydrogen, F, Cl, and methyl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and
R¹⁴ is selected from phenyl, halogen, hydroxy, C₁₋₂-alkyl, and hydrogen.

6. A compound of claim 1, wherein:
R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, and —O—CH—(CH₃)₂;
R₂ is selected from Cl, and F;
R₄ is A₆-L-R₉;
R₅ is selected from hydrogen, Cl, and methyl;
R₇ is selected from hydrogen, Cl, and methyl;
A₆ is NR₈;
L is selected from C₀₋₃-alkylene, —CD₂-, and C₃₋₈ branched alkylene;
R₈ is selected from hydrogen, and methyl; and
R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

7. A compound of claim 1, wherein:
R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, and —O—CH—(CH₃)₂;
R₂ is Cl;
R₄ is A₆-L-R₉;
R₅ is selected from hydrogen, and methyl;
R₇ is selected from hydrogen, and methyl;
A₆ is NR₈;
L is selected from —CH₂—, and —CD₂-;
R₈ is selected from hydrogen, and methyl; and
R₉ is selected from pyridyl, benzyl, tetrahydro-pyran, dioxane, and tetrahydrofuran, wherein said groups are optionally substituted with one to three substituents each independently selected from F, OH, methyl, ethyl, methoxy, and CN.

8. A compound of claim 1 selected from:
(S)-1-Methanesulfonyl-piperidine-3-carboxylic acid (5-chloro-4-{6-[(tetrahydro-pyran-4-ylmethyl)-amino]-pyrazin-2-yl}-pyridin-2-yl)-amide;
(S)-1-Ethanesulfonyl-piperidine-3-carboxylic acid (5-chloro-4-{6-[(tetrahydro-pyran-4-ylmethyl)-amino]-pyrazin-2-yl}-pyridin-2-yl)-amide;
(S)-1-(2-Methoxy-acetyl)-piperidine-3-carboxylic acid (5-chloro-4-{6-[(tetrahydro-pyran-4-ylmethyl)-amino]-pyrazin-2-yl}-pyridin-2-yl)-amide;
(S)-1-Acetyl-piperidine-3-carboxylic acid (5-chloro-4-{6-[(tetrahydro-pyran-4-ylmethyl)-amino]-pyrazin-2-yl}-pyridin-2-yl)-amide;
(1S,3R)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide;
(1R,3S)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide;
(1R,3R)-3-Methanesulfonylamino-cyclopentanecarboxylic acid (5-chloro-4-{6-[(tetrahydro-pyran-4-ylmethyl)-amino]-pyrazin-2-yl}-pyridin-2-yl)-amide;
[(1R,3S)-3-(5-Chloro-4-{6-[(tetrahydro-pyran-4-ylmethyl)-amino]-pyridin-2-yl}-pyridin-2-ylcarbamoyl)-cyclopentyl]-carbamic acid methyl ester;
[(1S,3R)-3-(5-Chloro-4-{6-[(tetrahydro-pyran-4-ylmethyl)-amino]-pyrazin-2-yl}-pyridin-2-ylcarbamoyl)-cyclopentyl]-carbamic acid methyl ester;
(S)-1-(Propane-2-sulfonyl)-piperidine-3-carboxylic acid (5-chloro-4-{6-[(tetrahydro-pyran-4-ylmethyl)-amino]-pyridin-2-yl}-pyridin-2-yl)-amide; and
1-Methyl-5-oxo-pyrrolidine-3-carboxylic acid (5-chloro-4-{6-[(tetrahydro-pyran-4-ylmethyl)-amino]-pyrazin-2-yl}-pyridin-2-yl)-amide.

9. A compound of claim 1 selected from:
(R)-Piperidine-3-carboxylic acid (5-chloro-4-{6-[(tetrahydro-pyran-4-ylmethyl)-amino]-pyrazin-2-yl}-pyridin-2-yl)-amide;
(R)-Piperidine-3-carboxylic acid {5-chloro-4-[6-(2-fluoro-benzylamino)-pyrazin-2-yl]-pyridin-2-yl}-amide;
Tetrahydro-pyran-4-carboxylic acid (5-chloro-4-{6-[(tetrahydro-pyran-4-ylmethyl)-amino]-pyrazin-2-yl}-pyridin-2-yl)-amide;
(R)-Piperidine-3-carboxylic acid (5-chloro-4-{6-[(5-fluoro-pyridin-3-ylmethyl)-amino]-pyrazin-2-yl}-pyridin-2-yl)-amide;
(R)-Piperidine-3-carboxylic acid {5-chloro-4-[6-(4-fluoro-benzylamino)-pyrazin-2-yl]-pyridin-2-yl}-amide;
Morpholine-2-carboxylic acid (5-chloro-4-{6-[(tetrahydro-pyran-4-ylmethyl)-amino]-pyrazin-2-yl}-pyridin-2-yl)-amide;
(R)-Piperidine-3-carboxylic acid (5-chloro-4-{3-methyl-6-[(tetrahydro-pyran-4-ylmethyl)-amino]-pyrazin-2-yl}-pyridin-2-yl)-amide;
(R)-Piperidine-3-carboxylic acid (5-chloro-4-{6-[(tetrahydro-pyran-3-ylmethyl)-amino]-pyrazin-2-yl}-pyridin-2-yl)-amide;
(R)-Piperidine-3-carboxylic acid {5-chloro-4-[6-((R)-1-cyclohexyl-ethylamino)-pyrazin-2-yl]-pyridin-2-yl}-amide;
(R)-Pyrrolidine-3-carboxylic acid (5-chloro-4-{6-[(tetrahydro-pyran-4-ylmethyl)-amino]-pyrazin-2-yl}-pyridin-2-yl)-amide;
(1R,3S)-3-Amino-cyclopentanecarboxylic acid (5-chloro-4 {6-[(tetrahydro-pyran-4-ylmethyl)-amino]-pyrazin-2-yl}-pyridin-2-yl)-amide;
(1R,3R)-3-Amino-cyclopentanecarboxylic acid (5-chloro-4-{6-[(tetrahydro-pyran-4-ylmethyl)-amino]-pyrazin-2-yl}-pyridin-2-yl)-amide; and
(3R,4S)-4-Fluoro-pyrrolidine-3-carboxylic acid (5-chloro-4-{6-[(tetrahydro-pyran-4-ylmethyl)-amino]-pyrazin-2-yl}-pyridin-2-yl)-amide.

10. (canceled)

11. (canceled)

12. A method of treatment of a disease or condition mediated by CDK9 comprising administration to a subject in need thereof a therapeutically effective amount of a compound according to claim 1, or a pharmaceutically acceptable salt thereof.

13. A pharmaceutical composition comprising a compound according to claim 1, or a pharmaceutically acceptable salt thereof, and a pharmaceutically acceptable carrier, diluent or excipient.

14. The compound of claim 1, wherein R₂ is Cl.

15. The compound of claim 14, wherein R₄ is -A₆-L-R₉, where R₉ is selected from pyridyl, benzyl, tetrahydro-pyran, dioxane, and tetrahydrofuran, wherein said R₉ group is optionally substituted with one to three substituents each independently selected from F, OH, methyl, ethyl, methoxy, and CN.

16. The compound of claim 15, wherein R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, and —O—CH—(CH₃)₂.
